(19) United States
(12) Patent Application Publication
(10) Pub. No.: US 2014/0315935 A1
(43) Pub. Date: Oct. 23, 2014

(54) GENE SIGNATURES FOR LUNG CANCER PROGNOSIS AND THERAPY SELECTION

(71) Applicant: Myriad Genetics, Inc., Salt Lake City, UT (US)

(72) Inventors: Susanne Wagner, Salt Lake City, UT (US); Steven Stone, Salt Lake City, UT (US); Alexander Gutin, Salt Lake City, UT (US); Julia Reid, Salt Lake City, UT (US)

(21) Appl. No.: 14/186,290

(22) Filed: Feb. 21, 2014

Related U.S. Application Data
(60) Provisional application No. 61/767,490, filed on Feb. 21, 2013.
(60) Provisional application No. 61/860,470, filed on Jul. 31, 2013.
(60) Provisional application No. 61/894,733, filed on Oct. 23, 2013.

(51) Int. Cl. C12Q 1/68

(52) U.S. Cl. CPC C12Q 1/6886
USPC 514/274; 506/9; 506/16

(57) ABSTRACT

The invention provides for molecular classification of disease and, particularly, molecular markers for lung cancer prognosis and therapy selection and methods and systems of use thereof.

### RELATED APPLICATIONS

This application claims the priority benefit of U.S. Provisional Application Ser. No. 61/767,490 (filed on Feb. 21, 2013), 61/860,470 (filed on Jul. 31, 2013), and 61/894,733 (filed on Oct. 23, 2013) all of which are hereby incorporated by reference in their entirety.

### FIELD OF THE INVENTION

The invention generally relates to a molecular classification of disease and particularly to molecular markers for lung cancer prognosis and therapy selection and methods of use thereof.

### TABLES

The instant application was filed with five (5) Tables under 37 C.F.R. §§1.52(e)(1)(iii) & 1.58(b), submitted electronically as the following text files:

a. Table A′:
i. File name: “3307-05-4P-2013-10-23-TABLEA′-BGJ.txt”
ii. Creation date: Jul. 30, 2013
iii. Size: 16,654 bytes

b. Table B′:
i. File name: “3307-05-3P-2013-07-31-TABLEB′-BGJ.txt”
ii. Creation date: Jul. 30, 2013
iii. Size: 196,290 bytes

c. Table C′:
i. File name: “3307-05-3P-2013-07-31-TABLEC′-BGJ.txt”
ii. Creation date: Jul. 30, 2013
iii. Size: 10,526 bytes

d. Table D′:
i. File name: “3307-05-3P-2013-07-31-TABLED′-BGJ.txt”
ii. Creation date: Jul. 30, 2013
iii. Size: 14,432 bytes

e. Table E′:
i. File name: “3307-05-3P-2013-07-31-TABLEE′-BGJ.txt”
ii. Creation date: Jul. 30, 2013
iii. Size: 13,720 bytes

LENGTHY TABLES
The patent application contains a lengthy table section. A copy of the table is available in electronic form from the USPTO web site (http://seqdata.uspto.gov/?pageRequest=docDetail&DocID=US20140315935A1). An electronic copy of the table will also be available from the USPTO upon request and payment of the fee set forth in 37 CFR 1.19(b)(3).

Each of the above files and all their contents are incorporated by reference herein in their entirety.

### BACKGROUND OF THE INVENTION

Cancer is a major public health problem, accounting for roughly 25% of all deaths in the United States. Though many treatments have been devised for various cancers, these treatments often vary in severity of side effects. It is useful for clinicians to know how aggressive a patient's cancer is in order to determine how aggressively to treat the cancer.

Early stage non small cell lung cancer (NSCLC) consists of the resectable stages IA, IB, IIA, IIB and IIIA. Stages are defined by tumor size and node involvement. Five year survival rates range from 70% in stage IA to 20% in stage IIIA. Multiple large scale adjuvant trials have found only a small benefit of adjuvant chemotherapy (4% improvement in survival rates) with most of the benefit centered in the higher stages. Current guidelines favor adjuvant treatment in stages II and III. In stage IA, however, treatment is counterindicated since the small benefit is often outweighed by the potential side effects. There are no recommendations for treatment of stage IB, although a fraction of IB patients is given adjuvant chemotherapy. Patients with stage IA or IB lung cancer are thus faced with a difficult decision of whether to undergo painful and expensive adjuvant chemotherapy or run the risk the cancer will recur after surgery. Price & Slevin, Difficult Decisions: Chemotherapy in Lung Cancer, POSTGRAD. MED. J. (1989) 65:291-298. Given the limited overall benefit of chemotherapy, the frequent co-morbidities in NSCLC patients and the frequent serious side effects of therapy, there is a serious need for novel and improved tools for predicting response to particular therapy regimens.

### SUMMARY OF THE INVENTION

The present invention is based in part on the surprising discovery that the expression of those genes whose expression closely tracks the cell cycle (“cell-cycle genes,” “CCGs,” or “CCP genes” as further defined below) is particularly useful in selecting appropriate therapy for and determining prognosis in lung cancer.

Accordingly, one aspect of the present invention provides a method for determining the prognosis and/or the likelihood of response to a particular treatment regimen in a patient having lung cancer, which comprises: determining in a sample from the patient the expression of a plurality of test genes comprising at least 6, 8 or 10 cell-cycle genes (e.g., genes in any of Tables 1-11 or Panels A-H, J, or K; “sub-panels” of Panel F in Tables A′ to E′), and correlating increased expression of said plurality of test genes to a poor prognosis and/or an increased likelihood of response to the particular treatment regimen (e.g., a treatment regimen comprising chemotherapy) or, optionally, (b) correlating no increased expression of said plurality of test genes to a good prognosis and/or no increased likelihood of response to the treatment regimen. In some embodiments the lung cancer is adenocarcinoma. In some embodiments the lung cancer is typical lung carcinoid. In some embodiments the lung cancer is atypical lung carcinoid.

In some embodiments, the plurality of test genes includes at least 8 cell-cycle genes, or at least 10, 15, 20, 25 or 30 cell-cycle genes (e.g., genes in any of Tables 1-11 or Panels A-H, J, or K; “sub-panels” of Panel F in Tables A′ to E′). In some embodiments, at least some proportion of the test genes (e.g., at least 10%, 15%, 20%, 25%, 30%, 40%, 50%, 60%, 70%, 80%, 85%, 90%, 95%, or 99%) are cell-cycle genes. In some embodiments, all of the test genes are cell-cycle genes.

In some embodiments, the step of determining the expression of the plurality of test genes in the tumor sample comprises measuring the amount of mRNA in the tumor sample transcribed from each of from 6 to about 200 cell-cycle genes; and measuring the amount of mRNA of one or more housekeeping genes in the tumor sample.

In one embodiment, the method of determining the prognosis and/or the likelihood of response to a particular treatment regimen comprises (1) determining in a tumor sample from a patient having lung cancer the expression of a panel of genes in said tumor sample including at least 4 or at least 8 cell-cycle genes (e.g., genes in any of Tables 1-11 or Panels A-H, J, or K; “sub-panels” of Panel F in Tables A′ to E′); (2) providing a test value by (a) weighting the determined expression of each of a plurality of test genes selected from the panel of genes with a predefined coefficient, and (b) combining the weighted expression to provide the test value, wherein at least 50%, at least 75% or at least 85% of the plurality of test genes are cell-cycle genes; and (3)(a) correlating an increased level of overall expression of the plurality of test genes to a poor prognosis and/or an increased likelihood of response to the particular treatment regimen (e.g., a treatment regimen comprising chemotherapy), or (b) correlating no increase in the overall expression of the test genes to a good prognosis and/or no increased likelihood of response to the treatment regimen. In some embodiments the lung cancer is adenocarcinoma. In some embodiments the lung cancer is typical lung carcinoid. In some embodiments the lung cancer is atypical lung carcinoid.

In some embodiments, the methods of the invention further include a step of comparing the test value provided in step (2) above to one or more reference values, and correlating the test value to an increased likelihood of response to the particular treatment regimen. Optionally a test value greater than the reference value is correlated to an increased likelihood of response to treatment comprising chemotherapy. In some embodiments the test value is correlated to an increased likelihood of response to treatment (e.g., treatment comprising chemotherapy) if the test value exceeds the reference value by at least some amount (e.g., at least 0.5, 0.75, 0.85, 0.90, 0.95, 1, 2, 3, 4, 5, 6, 7, 8, 9, or 10 or more fold or standard deviations).

In some embodiments, the method of determining the likelihood of response to a particular treatment regimen comprises (1) determining in a tumor sample from a patient having lung cancer the expression of a panel of genes in said tumor sample including at least 4 or at least 8 cell-cycle genes (e.g., genes in any of Tables 1-11 or Panels A-H, J, or K; “sub-panels” of Panel F in Tables A′ to E′); (2) providing a test value by (a) weighting the determined expression of each of a plurality of test genes selected from the panel of genes with a predefined coefficient, and (b) combining the weighted expression to provide the test value, wherein the cell-cycle genes are weighted to contribute at least 50%, at least 75% or at least 85% of the test value; and (3)(a) correlating a test value that is greater than some reference to a poor prognosis and/or an increased likelihood of response to the particular treatment regimen (e.g., a treatment regimen comprising chemotherapy), or (b) correlating a test value that is not greater than the reference to a good prognosis and/or no increased likelihood of response to the treatment.

In another aspect, the present invention provides a method of treating cancer in a patient identified as having lung cancer, comprising: (1) determining in a tumor sample from the patient the expression of a panel of genes in the tumor sample including at least 4 or at least 8 cell-cycle genes (e.g., genes in any of Tables 1-11 or Panels A-H, J, or K; “sub-panels” of Panel F in Tables A′ to E′); (2) providing a test value by (a) weighting the determined expression of each of a plurality of test genes selected from said panel of genes with a predefined coefficient, and (b) combining the weighted expression to provide said test value, wherein the cell-cycle genes are weighted to contribute at least 50%, at least 75% or at least 85% of the test value; (3)(a) correlating an increased level of overall expression of the plurality of test genes to a poor prognosis and/or an increased likelihood of response to a particular treatment regimen (e.g., a treatment regimen comprising chemotherapy), or (b) correlating no increase in the overall expression of the test genes to a good prognosis and/or no increased likelihood of response to the treatment; and (4) recommending, prescribing or administering a particular treatment regimen (e.g., a treatment regimen comprising chemotherapy) based at least in part on the result in step (3). In some embodiments the lung cancer is adenocarcinoma. In some embodiments the lung cancer is typical lung carcinoid. In some embodiments the lung cancer is atypical lung carcinoid

The present invention further provides a diagnostic kit for determining the prognosis in a patient having lung cancer and/or predicting the likelihood of response to a particular treatment regimen (e.g., a treatment regimen comprising chemotherapy) in a patient having lung cancer, comprising, in a compartmentalized container, a plurality of oligonucleotides hybridizing to at least 8 test genes, wherein less than 10%, 30% or less than 40% of all of the at least 8 test genes are non-cell-cycle genes; and one or more oligonucleotides hybridizing to at least one housekeeping gene. The oligonucleotides can be hybridizing probes for hybridization with the test genes under stringent conditions or primers suitable for PCR amplification of the test genes. In one embodiment, the kit consists essentially of, in a compartmentalized container, a first plurality of PCR reaction mixtures for PCR amplification of from 5 or 10 to about 300 test genes, wherein at least 30% or 50%, at least 60% or at least 80% of such test genes are cell-cycle genes (e.g., genes in any of Tables 1-11 or Panels A-H, J, or K; “sub-panels” of Panel F in Tables A′ to E′), and wherein each reaction mixture comprises a PCR primer pair for PCR amplifying one of the test genes; and a second plurality of PCR reaction mixtures for PCR amplification of at least one control (e.g., housekeeping) gene. In some embodiments the kit comprises one or more computer software programs for calculating a test value representing the expression of the test genes (either the overall expression of all test genes or of some subset) and for comparing this test value to some reference value. In some embodiments such computer software is programmed to weight the test genes such that cell-cycle genes are weighted to contribute at least 50%, at least 75% or at least 85% of the test value. In some embodiments such computer software is programmed to communicate (e.g., display) that the patient has an increased likelihood of response to a treatment regimen comprising chemotherapy if the test value is greater than the reference value (e.g., by more than some predetermined amount).

The present invention also provides the use of (1) a plurality of oligonucleotides hybridizing to at least 4 or at least 8 cell-cycle genes (e.g., genes in any of Tables 1-11 or Panels A-H, J, or K; “sub-panels” of Panel F in Tables A′ to E′); and (2) one or more oligonucleotides hybridizing to at least one control (e.g., housekeeping) gene, for the manufacture of a diagnostic product for determining the expression of the test genes in a tumor sample from a patient having lung cancer, to determine prognosis in said patient and/or to predict the likelihood of responding to a treatment regimen comprising chemotherapy, wherein an increased level of the overall expression of the test genes indicates an increased likelihood, whereas no increase in the overall expression of the test genes indicates no increased likelihood. In some embodiments, the oligonucleotides are PCR primers suitable for PCR amplification of the test genes. In other embodiments, the oligonucleotides are probes hybridizing to the test genes under stringent conditions. In some embodiments, the plurality of oligonucleotides are probes for hybridization under stringent conditions to, or are suitable for PCR amplification of, from 4 to about 300 test genes, at least 50%, 70% or 80% or 90% of the test genes being cell-cycle genes. In some other embodiments, the plurality of oligonucleotides are hybridization probes for, or are suitable for PCR amplification of, from 20 to about 300 test genes, at least 30%, 40%, 50%, 70% or 80% or 90% of the test genes being cell-cycle genes.

The present invention further provides a system for determining the prognosis in a patient having lung cancer and/or the likelihood of response to a particular treatment regimen in a patient having lung cancer, comprising: (1) a sample analyzer for determining the expression levels of a panel of genes in a tumor sample including at least 4 cell-cycle genes (e.g., genes in any of Tables 1-11 or Panels A-H, J, or K; “sub-panels” of Panel F in Tables A′ to E′), wherein the sample analyzer contains the tumor sample, mRNA molecules expressed from the panel of genes and extracted from the sample, or cDNA molecules from said mRNA molecules; (2) a first computer program for (a) receiving gene expression data on at least 4 test genes selected from the panel of genes, (b) weighting the determined expression of each of the test genes with a predefined coefficient, and (c) combining the weighted expression to provide a test value, wherein at least 50%, at least at least 75% of at least 4 test genes are cell-cycle genes; and (3) a second computer program for comparing the test value to one or more reference values each associated with a predetermined prognosis or likelihood of response to the particular treatment.

In some embodiments the invention provides a system for determining the prognosis in a patient having lung cancer and/or the likelihood of response to a particular treatment regimen in a patient having lung cancer, comprising: (1) a sample analyzer for determining the expression levels of a panel of genes in a tumor sample including at least 4 cell-cycle genes (e.g., genes in any of Tables 1-11 or Panels A-H, J, or K; “sub-panels” of Panel F in Tables A′ to E′), wherein the sample analyzer contains the tumor sample, mRNA molecules expressed from the panel of genes and extracted from the sample, or cDNA molecules from said mRNA molecules; (2) a first computer program for (a) receiving gene expression data on at least 4 test genes selected from the panel of genes, (b) weighting the determined expression of each of the test genes with a predefined coefficient, and (c) combining the weighted expression to provide a test value, wherein the cell-cycle genes are weighted to contribute at least 50%, at least 75% or at least 85% of the test value; and (3) a second computer program for comparing the test value to one or more reference values each associated with a predetermined prognosis or likelihood of response to the particular treatment regimen (e.g., a treatment regimen comprising chemotherapy). In some embodiments, the system further comprises a display module displaying the comparison between the test value and the one or more reference values, or displaying a result of the comparing step.

Unless otherwise defined, all technical and scientific terms used herein have the same meaning as commonly understood by one of ordinary skill in the art to which this invention pertains. Although methods and materials similar or equivalent to those described herein can be used in the practice or testing of the present invention, suitable methods and materials are described below. In case of conflict, the present specification, including definitions, will control. In addition, the materials, methods, and examples are illustrative only and not intended to be limiting.

Other features and advantages of the invention will be apparent from the following Detailed Description, and from the Claims.

### BRIEF DESCRIPTION OF THE DRAWINGS

FIG. 1 is a Kaplan Meier plot of clinical sample set 1, stage I and II, using CCP score quartiles and disease survival as outcome measure.

FIG. 2 is Kaplan Meier plot of clinical sample set lstage IB only, using the CCP mean to separate a high CCP from a low CCP group and disease survival as outcome measure.

FIG. 3 shows the distribution of CCP scores in two independent stage IB cohorts.

FIG. 4 is a Kaplan Meier survival analysis of CCP score in the combined stage IB samples of set 1 and set 2.

FIG. 5 is a Kaplan Meier survival analysis of CCP and treatment in combined stage IB samples.

FIG. 6 is an illustration of an example of a system useful in certain aspects and embodiments of the invention.

FIG. 7 is a flowchart illustrating an example of a computer-implemented method of the invention.

FIG. 8 is an illustration of the predictive power for CCG panels of different sizes.

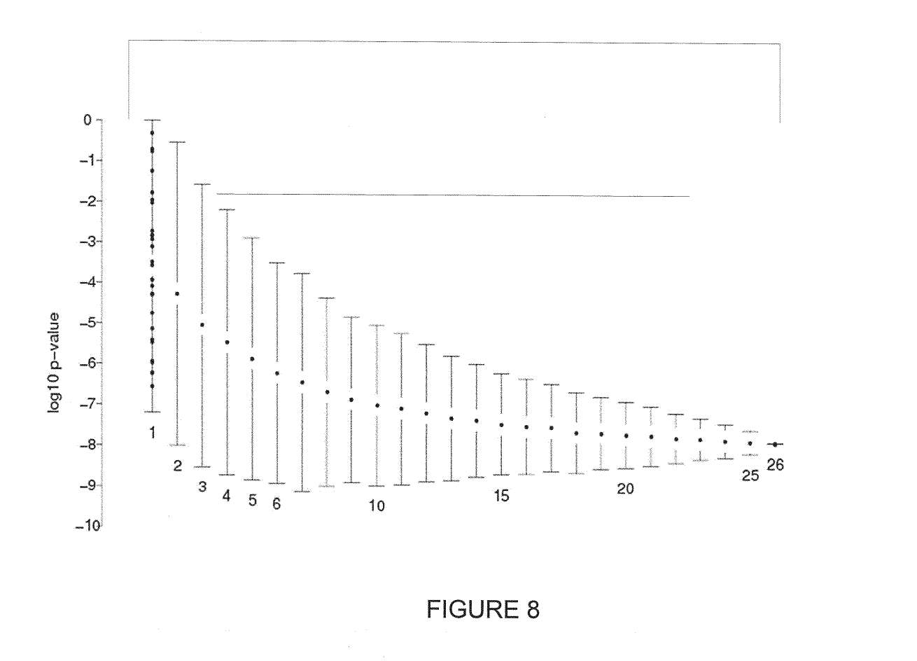

FIG. 9 shows the distribution of CCP scores in the Combined Cohort of Example 2.

FIG. 10 is a Kaplan Meier survival analysis of CCP score in the Combined Cohort of Example 2.

FIG. 11 shows how CCP score predicts treatment benefit in Example 3.

FIG. 12 shows the consistency of hazard ratios for CCP score across cohorts.

FIG. 13 shows the consistency of hazard ratios for pathological stage across cohorts.

FIG. 14 shows predicted 5-year disease mortality risk as a function of Prognostic Score (as shown in the training study in Example 4).

FIG. 15 shows 5-year disease mortality risk as predicted by Prognostic Score versus as predicted by pathological stage alone (as shown in the training study in Example 4).

FIG. 16 shows predicted 5-year disease mortality risk as a function of Prognostic Score (as shown in the validation study in Example 4), with a cut-off value of PS=27 as a divider in one embodiment between low risk and high risk patients.

FIG. 17 is a Kaplan Meier survival analysis of Prognostic Score (as shown in the validation study in Example 4).

FIG. 18 shows 5-year disease mortality risk as predicted by Prognostic Score versus as predicted by pathological stage alone (as shown in the validation study in Example 4).

### DETAILED DESCRIPTION OF THE INVENTION

The present invention is based in part on the discovery that genes whose expression closely tracks the cell cycle (“cell-cycle genes” or “CCGs”) are particularly powerful genes for classifying lung cancer, including determining prognosis and/or the likelihood a particular patient will respond to a particular treatment regimen (e.g., a regimen comprising chemotherapy).

“Cell-cycle gene” and “CCG” herein refer to a gene whose expression level closely tracks the progression of the cell through the cell-cycle. See, e.g., Whitfield et al., MOL. BIOL. CELL (2002) 13:1977-2000. The term “cell-cycle progression” or “CCP” will also be used in this application and will generally be interchangeable with CCG (i.e., a CCP gene is a CCG; a CCP score is a CCG score). More specifically, CCGs show periodic increases and decreases in expression that coincide with certain phases of the cell cycle—e.g., STK15 and PLK show peak expression at G2/M. Id. Often CCGs have clear, recognized cell-cycle related function—e.g., in DNA synthesis or repair, in chromosome condensation, in cell-division, etc. However, some CCGs have expression levels that track the cell-cycle without having an obvious, direct role in the cell-cycle—e.g., UBE2S encodes a ubiquitin-conjugating enzyme, yet its expression closely tracks the cell-cycle. Thus a CCG according to the present invention need not have a recognized role in the cell-cycle. Exemplary CCGs are listed in Tables 1, 2, 3, 5, 6, 7, 8 & 9. A fuller discussion of CCGs, including an extensive (though not exhaustive) list of CCGs, can be found in International Application No. PCT/US2010/020397 (pub. no. WO/2010/080933) (see, e.g., Table 1 in WO/2010/080933). International Application No. PCT/US2010/020397 (pub. no. WO/2010/080933 (see also corresponding U.S. application Ser. No. 13/177,887)) and International Application No. PCT/US2011/043228 (pub no. WO/2012/006447 (see also related U.S. application Ser. No. 13/178,380)) and their contents are hereby incorporated by reference in their entirety.

Whether a particular gene is a CCG may be determined by any technique known in the art, including those taught in Whitfield et al., MOL. BIOL. CELL (2002) 13:1977-2000; Whitfield et al., MOL. CELL. BIOL. (2000) 20:4188-4198; WO/2010/080933 (¶[0039]). All of the CCGs in Table 1 below form a panel of CCGs (“Panel A”) useful in the invention. As will be shown detail throughout this document, individual CCGs (e.g., CCGs in Table 1) and subsets of these genes can also be used in the invention.

TABLE 1

| | Entrez | | RefSeq Accession |
|---|---|---|---|
| Gene Symbol | GeneID | ABI Assay ID | Nos. |
| APOBEC3B* | 9582 | Hs00358981_m1 | NM_004900.3 |
| ASF1B* | 55723 | Hs00216780_m1 | NM_018154.2 |
| ASPM* | 259266 | Hs00411505_m1 | NM_018136.4 |
| ATAD2* | 29028 | Hs00204205_m1 | NM_014109.3 |
| BIRC5* | 332 | Hs00153353_m1; | NM_001012271.1; |
| | | Hs03043576_m1 | NM_001012270.1; |
| | | | NM_001168.2 |
| BLM* | 641 | Hs00172060_m1 | NM_000057.2 |
| BUB1 | 699 | Hs00177821_m1 | NM_004336.3 |
| BUB1B* | 701 | Hs01084828_m1 | NM_001211.5 |
| C12orf48* | 55010 | Hs00215575_m1 | NM_017915.2 |
| C18orf24* | 220134 | Hs00536843_m1 | NM_145060.3; |
| | | | NM_001039535.2 |
| C1orf135* | 79000 | Hs00225211_m1 | NM_024037.1 |
| C21orf45* | 54069 | Hs00219050_m1 | NM_018944.2 |
| CCDC99* | 54908 | Hs00215019_m1 | NM_017785.4 |
| CCNA2* | 890 | Hs00153138_m1 | NM_001237.3 |
| CCNB1* | 891 | Hs00259126_m1 | NM_031966.2 |
| CCNB2* | 9133 | Hs00270424_m1 | NM_004701.2 |
| CCNE1* | 898 | Hs01026536_m1 | NM_001238.1; |
| | | | NM_057182.1 |
| CDC2* | 983 | Hs00364293_m1 | NM_033379.3; |
| | | | NM_001130829.1; |
| | | | NM_001786.3 |
| CDC20* | 991 | Hs03004916_g1 | NM_001255.2 |
| CDC45L* | 8318 | Hs00185895_m1 | NM_003504.3 |
| CDC6* | 990 | Hs00154374_m1 | NM_001254.3 |
| CDCA3* | 83461 | Hs00229905_m1 | NM_031299.4 |
| CDCA8* | 55143 | Hs00983655_m1 | NM_018101.2 |
| CDKN3* | 1033 | Hs00193192_m1 | NM_001130851.1; |
| | | | NM_005192.3 |
| CDT1* | 81620 | Hs00368864_m1 | NM_030928.3 |
| CENPA | 1058 | Hs00156455_m1 | NM_001042426.1; |
| | | | NM_001809.3 |
| CENPE* | 1062 | Hs00156507_m1 | NM_001813.2 |
| CENPF* | 1063 | Hs00193201_m1 | NM_016343.3 |
| CENPI* | 2491 | Hs00198791_m1 | NM_006733.2 |
| CENPM* | 79019 | Hs00608780_m1 | NM_024053.3 |
| CENPN* | 55839 | Hs00218401_m1 | NM_018455.4; |
| | | | NM_001100624.1; |
| | | | NM_001100625.1 |
| CEP55* | 55165 | Hs00216688_m1 | NM_018131.4; |
| | | | NM_001127182.1 |
| CHEK1* | 1111 | Hs00967506_m1 | NM_001114121.1; |
| | | | NM_001114122.1; |
| | | | NM_001274.4 |
| CKAP2* | 26586 | Hs00217068_m1 | NM_018204.3; |
| | | | NM_001098525.1 |
| CKS1B* | 1163 | Hs01029137_g1 | NM_001826.2 |
| CKS2* | 1164 | Hs01048812_g1 | NM_001827.1 |
| CTPS* | 1503 | Hs01041851_m1 | NM_001905.2 |
| CTSL2* | 1515 | Hs00952036_m1 | NM_001333.2 |
| DBF4* | 10926 | Hs00272696_m1 | NM_006716.3 |
| DDX39* | 10212 | Hs00271794_m1 | NM_005804.2 |
| DLGAP5/ | 9787 | Hs00207323_m1 | NM_014750.3 |
| DLG7* | | | |
| DONSON* | 29980 | Hs00375083_m1 | NM_017613.2 |
| DSN1* | 79980 | Hs00227760_m1 | NM_024918.2 |
| DTL* | 51514 | Hs00978565_m1 | NM_016448.2 |
| E2F8* | 79733 | Hs00226635_m1 | NM_024680.2 |
| ECT2* | 1894 | Hs00216455_m1 | NM_018098.4 |
| ESPL1* | 9700 | Hs00202246_m1 | NM_012291.4 |
| EXO1* | 9156 | Hs00243513_m1 | NM_130398.2; |
| | | | NM_003686.3; |
| | | | NM_006027.3 |
| EZH2* | 2146 | Hs00544830_m1 | NM_152998.1; |
| | | | NM_004456.3 |
| FANCI* | 55215 | Hs00289551_m1 | NM_018193.2; |
| | | | NM_001113378.1 |
| FBXO5* | 26271 | Hs03070834_m1 | NM_001142522.1; |
| | | | NM_012177.3 |
| FOXM1* | 2305 | Hs01073586_m1 | NM_202003.1; |
| | | | NM_202002.1; |
| | | | NM_021953.2 |
| GINS1* | 9837 | Hs00221421_m1 | NM_021067.3 |
| GMPS* | 8833 | Hs00269500_m1 | NM_003875.2 |
| GPSM2* | 29899 | Hs00203271_m1 | NM_013296.4 |
| GTSE1* | 51512 | Hs00212681_m1 | NM_016426.5 |
| H2AFX* | 3014 | Hs00266783_s1 | NM_002105.2 |
| HMMR* | 3161 | Hs00234864_m1 | NM_001142556.1; |
| | | | NM_001142557.1; |
| | | | NM_012484.2; |
| | | | NM_012485.2 |
| HN1* | 51155 | Hs00602957_m1 | NM_001002033.1; |
| | | | NM_001002032.1; |
| | | | NM_016185.2 |
| KIAA0101* | 9768 | Hs00207134_m1 | NM_014736.4 |
| KIF11* | 3832 | Hs00189698_m1 | NM_004523.3 |
| KIF15* | 56992 | Hs00173349_m1 | NM_020242.2 |
| KIF18A* | 81930 | Hs01015428_m1 | NM_031217.3 |
| KIF20A* | 10112 | Hs00993573_m1 | NM_005733.2 |
| KIF20B/ | 9585 | Hs01027505_m1 | NM_016195.2 |
| MPHOSPH1* | | | |
| KIF23* | 9493 | Hs00370852_m1 | NM_138555.1; |
| | | | NM_004856.4 |
| KIF2C* | 11004 | Hs00199232_m1 | NM_006845.3 |
| KIF4A* | 24137 | Hs01020169_m1 | NM_012310.3 |
| KIFC1* | 3833 | Hs00954801_m1 | NM_002263.3 |
| KPNA2 | 3838 | Hs00818252_g1 | NM_002266.2 |
| LMNB2* | 84823 | Hs00383326_m1 | NM_032737.2 |
| MAD2L1 | 4085 | Hs01554513_g1 | NM_002358.3 |
| MCAM* | 4162 | Hs00174838_m1 | NM_006500.2 |
| MCM10* | 55388 | Hs00960349_m1 | NM_018518.3; |
| | | | NM_182751.1 |
| MCM2* | 4171 | Hs00170472_m1 | NM_004526.2 |
| MCM4* | 4173 | Hs00381539_m1 | NM_005914.2; |
| | | | NM_182746.1 |
| MCM6* | 4175 | Hs00195504_m1 | NM_005915.4 |
| MCM7* | 4176 | Hs01097212_m1 | NM_005916.3; |
| | | | NM_182776.1 |
| MELK | 9833 | Hs00207681_m1 | NM_014791.2 |
| MKI67* | 4288 | Hs00606991_m1 | NM_002417.3 |
| MYBL2* | 4605 | Hs00231158_m1 | NM_002466.2 |
| NCAPD2* | 9918 | Hs00274505_m1 | NM_014865.3 |
| NCAPG* | 64151 | Hs00254617_m1 | NM_022346.3 |
| NCAPG2* | 54892 | Hs00375141_m1 | NM_017760.5 |
| NCAPH* | 23397 | Hs01010752_m1 | NM_015341.3 |
| NDC80* | 10403 | Hs00196101_m1 | NM_006101.2 |
| NEK2* | 4751 | Hs00601227_mH | NM_002497.2 |
| NUSAP1* | 51203 | Hs01006195_m1 | NM_018454.6; |
| | | | NM_001129897.1; |
| | | | NM_016359.3 |
| OIP5* | 11339 | Hs00299079_m1 | NM_007280.1 |
| ORC6L* | 23594 | Hs00204876_m1 | NM_014321.2 |
| PAICS* | 10606 | Hs00272390_m1 | NM_001079524.1; |
| | | | NM_001079525.1; |
| | | | NM_006452.3 |
| PBK* | 55872 | Hs00218544_m1 | NM_018492.2 |
| PCNA* | 5111 | Hs00427214_g1 | NM_182649.1; |
| | | | NM_002592.2 |
| PDSS1* | 23590 | Hs00372008_m1 | NM_014317.3 |
| PLK1* | 5347 | Hs00153444_m1 | NM_005030.3 |
| PLK4* | 10733 | Hs00179514_m1 | NM_014264.3 |
| POLE2* | 5427 | Hs00160277_m1 | NM_002692.2 |
| PRC1* | 9055 | Hs00187740_m1 | NM_199413.1; |
| | | | NM_199414.1; |
| | | | NM_003981.2 |
| PSMA7* | 5688 | Hs00895424_m1 | NM_002792.2 |
| PSRC1* | 84722 | Hs00364137_m1 | NM_032636.6; |
| | | | NM_001005290.2; |
| | | | NM_001032290.1; |
| | | | NM_001032291.1 |
| PTTG1* | 9232 | Hs00851754_u1 | NM_004219.2 |
| RACGAP1* | 29127 | Hs00374747_m1 | NM_013277.3 |
| RAD51* | 5888 | Hs00153418_m1 | NM_133487.2; |
| | | | NM_002875.3 |
| RAD51AP1* | 10635 | Hs01548891_m1 | NM_001130862.1; |
| | | | NM_006479.4 |
| RAD54B* | 25788 | Hs00610716_m1 | NM_012415.2 |
| RAD54L* | 8438 | Hs00269177_m1 | NM_001142548.1; |
| | | | NM_003579.3 |
| RFC2* | 5982 | Hs00945948_m1 | NM_181471.1; |
| | | | NM_002914.3 |
| RFC4* | 5984 | Hs00427469_m1 | NM_181573.2; |
| | | | NM_002916.3 |
| RFC5* | 5985 | Hs00738859_m1 | NM_181578.2; |
| | | | NM_001130112.1; |
| | | | NM_001130113.1; |
| | | | NM_007370.4 |
| RNASEH2A* | 10535 | Hs00197370_m1 | NM_006397.2 |
| RRM2* | 6241 | Hs00357247_g1 | NM_001034.2 |
| SHCBP1* | 79801 | Hs00226915_m1 | NM_024745.4 |
| SMC2* | 10592 | Hs00197593_m1 | NM_001042550.1; |
| | | | NM_001042551.1; |
| | | | NM_006444.2 |
| SPAG5* | 10615 | Hs00197708_m1 | NM_006461.3 |
| SPC25* | 57405 | Hs00221100_m1 | NM_020675.3 |
| STIL* | 6491 | Hs00161700_m1 | NM_001048166.1; |
| | | | NM_003035.2 |
| STMN1* | 3925 | Hs00606370_m1; | NM_005563.3; |
| | | Hs01033129_m1 | NM_203399.1 |
| TACC3* | 10460 | Hs00170751_m1 | NM_006342.1 |
| TIMELESS* | 8914 | Hs01086966_m1 | NM_003920.2 |
| TK1* | 7083 | Hs01062125_m1 | NM_003258.4 |
| TOP2A* | 7153 | Hs00172214_m1 | NM_001067.2 |
| TPX2* | 22974 | Hs00201616_m1 | NM_012112.4 |
| TRIP13* | 9319 | Hs01020073_m1 | NM_004237.2 |
| TTK* | 7272 | Hs00177412_m1 | NM_003318.3 |
| TUBA1C* | 84790 | Hs00733770_m1 | NM_032704.3 |
| TYMS* | 7298 | Hs00426591_m1 | NM_001071.2 |
| UBE2C | 11065 | Hs00964100_g1 | NM_181799.1; |
| | | | NM_181800.1; |
| | | | NM_181801.1; |
| | | | NM_181802.1; |
| | | | NM_181803.1; |
| | | | NM_007019.2 |
| UBE2S | 27338 | Hs00819350_m1 | NM_014501.2 |
| VRK1* | 7443 | Hs00177470_m1 | NM_003384.2 |
| ZWILCH* | 55055 | Hs01555249_m1 | NM_017975.3; |
| | | | NR_003105.1 |
| ZWINT* | 11130 | Hs00199952_m1 | NM_032997.2; |
| | | | NM_001005413.1; |
| | | | NM_007057.3 |

*124-gene subset of CCGs useful in the invention (“Panel B”). ABI Assay ID means the catalogue ID number for the gene expression assay commercially available from Applied Biosystems Inc. (Foster City, CA) for the particular gene.

As shown in Examples 1 & 2 below, it has been surprisingly discovered that patients whose tumors show increased expression of CCGs (e.g., a CCP score or test value reflecting higher CCP gene expression) have poorer prognosis, yet respond better to treatment comprising chemotherapy, than patients whose tumors do not show such an increase. Accordingly, one aspect of the present invention provides a method for determining the prognosis in a patient having lung cancer and/or the likelihood of response to a particular treatment regimen in a patient having lung cancer, which comprises: determining in a tumor sample from the patient the expression of a plurality of test genes comprising at least 2, 3, 4, 5, 6, 7 or at least 8, 9, 10 or 12 cell-cycle genes (e.g., genes in any of Tables 1-11 or Panels A-H, J, or K; “sub-panels” of Panel F in Tables A′ to E′), and correlating increased expression of said plurality of test genes to a poor prognosis and/or an increased likelihood of response to the particular treatment regimen (e.g., a treatment regimen comprising chemotherapy).

The embodiments of the invention described herein involve lung cancer. Lung cancer as used herein includes at least adenocarcinoma, atypical lung carcinoids, and typical lung carcinoids.

Several embodiments of the invention described herein involve a step of correlating high CCP gene expression according to the present invention (e.g., high expression of a panel of CCP genes as described in various embodiments throughout this application; a test value derived from or reflecting high expression of such a panel; etc.) to a particular clinical feature (e.g., a poor prognosis; an increased likelihood of lung cancer recurrence; an increased likelihood of response to chemotherapy; etc.) if the CCP gene expression is greater than some reference (or optionally to another feature, e.g., good prognosis, if the expression is less than some reference). Throughout this document, wherever such an embodiment is described, a further, related embodiment of the invention may involve, in addition to or instead of a correlating step, one or both of the following steps: (a) concluding that the patient has (or classifying the patient as having) the clinical feature based at least in part on high CCP expression (or a test value derived from or reflecting such); or (b) communicating that the patient has the clinical feature based at least in part on high CCP expression (or a test value derived from or reflecting such).

By way of illustration, but not limitation, one embodiment described in this document is a method for determining in a patient the prognosis of lung cancer or the likelihood of such a patient to respond to chemotherapy, comprising: (1) determining the expression of a plurality of test genes comprising at least 2, 3, 4, 5, 6, 7, 8, 9, 10 or 15 or more cell-cycle genes (e.g., CCGs in Panel F; in any of Panels H, I, J, L, M, N & O; or in any sub-panel of Panel F in any of Tables A′ through E′; etc.), and (2) correlating high expression of said plurality of test genes to poor prognosis of the lung cancer in the patient or an increased likelihood of response to chemotherapy. According to the preceding paragraph, this description of this embodiment is understood to include a description of two further, related embodiments, i.e., a method for determining in a patient the prognosis of lung cancer or the likelihood of such a patient to respond to chemotherapy, comprising: (1) determining the expression of a plurality of test genes comprising at least 2, 3, 4, 5, 6, 7, 8, 9, 10 or 15 or more cell-cycle genes (e.g., CCGs in Panel F; in any of Panels H, I, J, L, M, N & O; or in any sub-panel of Panel F in any of Tables A′ through E′; etc.), and (2)(a) concluding that said patient has a poor prognosis of the lung cancer in the patient or an increased likelihood of response to chemotherapy based at least in part on high expression of said plurality of test genes; or (2)(b) communicating that said patient has a poor prognosis of the lung cancer in the patient or an increased likelihood of response to chemotherapy based at least in part on high expression of said plurality of test genes.

In each embodiment described in this document involving correlating a particular assay or analysis output (e.g., high CCG expression, test value incorporating CCG expression greater than some reference value, etc.) to some likelihood (e.g., increased, not increased, decreased, etc.) of some clinical event or outcome (e.g., recurrence, progression, cancer-specific death, etc.), such correlating may comprise assigning a risk or likelihood of the clinical event or outcome occurring based at least in part on the particular assay or analysis output. In some embodiments, such risk is a percentage probability of the event or outcome occurring. In some embodiments, the patient is assigned to a risk group (e.g., low risk, intermediate risk, high risk, etc.). In some embodiments “low risk” is any percentage probability below 5%, 10%, 15%, 20%, 25%, 30%, 35%, 40%, 45%, or 50%. In some embodiments “intermediate risk” is any percentage probability above 5%, 10%, 15%, 20%, 25%, 30%, 35%, 40%, 45%, or 50% and below 15%, 20%, 25%, 30%, 35%, 40%, 45%, 50%, 55%, 60%, 65%, 70%, or 75%. In some embodiments “high risk” is any percentage probability above 25%, 30%, 35%, 40%, 45%, 50%, 55%, 60%, 65%, 70%, 75%, 80%, 85%, 90%, 95%, or 99%.

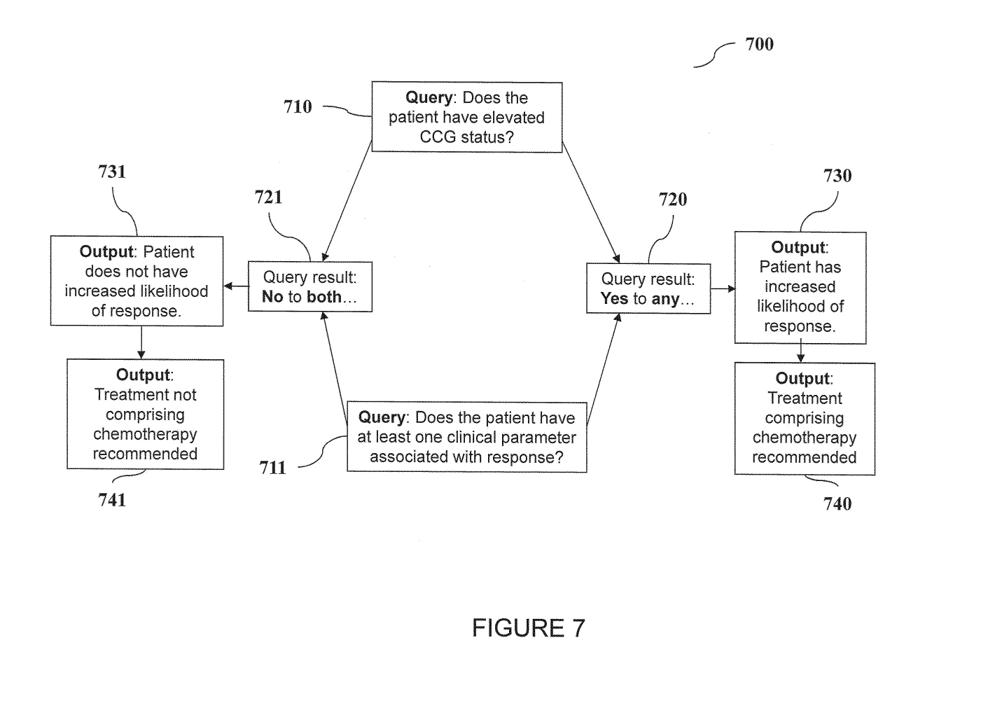

As used herein, “communicating” a particular piece of information means to make such information known to another person or transfer such information to a thing (e.g., a computer). In some methods of the invention, a patient's prognosis or risk of recurrence is communicated. In some embodiments, the information used to arrive at such a prognosis or risk prediction (e.g., expression levels of a panel of biomarkers comprising a plurality of CCGs, clinical or pathologic factors, etc.) is communicated. This communication may be auditory (e.g., verbal), visual (e.g., written), electronic (e.g., data transferred from one computer system to another), etc. In some embodiments, communicating a cancer classification comprises generating a report that communicates the cancer classification. In some embodiments the report is a paper report, an auditory report, or an electronic record. In some embodiments the report is displayed and/or stored on a computing device (e.g., handheld device, desktop computer, smart device, website, etc.). In some embodiments the cancer classification is communicated to a physician (e.g., a report communicating the classification is provided to the physician). In some embodiments the cancer classification is communicated to a patient (e.g., a report communicating the classification is provided to the patient). Communicating a cancer classification can also be accomplished by transferring information (e.g., data) embodying the classification to a server computer and allowing an intermediary or end-user to access such information (e.g., by viewing the information as displayed from the server, by downloading the information in the form of one or more files transferred from the server to the intermediary or end-user's device, etc.).

Wherever an embodiment of the invention comprises concluding some fact (e.g., a patient's prognosis or a patient's likelihood of recurrence), this may include a computer program concluding such fact, typically after performing some algorithm that incorporates information on the status of CCGs in a patient sample (e.g., as shown in FIG. 7).

In some embodiments, determining the expression of a plurality of genes comprises receiving a report communicating such expression. In some embodiments this report communicates such expression in a qualitative manner (e.g., “high” or “increased”). In some embodiments this report communicates such expression indirectly by communicating a score (e.g., prognosis score, recurrence score, etc.) that incorporates such expression.

In some embodiments, the method includes (1) obtaining a sample from a patient having lung cancer; (2) determining the expression of a panel of genes in the tumor sample including at least 2, 4, 5, 6, 7 or at least 8, 9, 10 or 12 cell-cycle genes (e.g., genes in any of Tables 1-11 or Panels A-H, J, or K; “sub-panels” of Panel F in Tables A′ to E′); (3) providing a test value by (a) weighting the determined expression of each of a plurality of test genes selected from the panel of genes with a predefined coefficient, and (b) combining the weighted expression to provide said test value, wherein at least 20%, at least 50%, at least 75% or at least 90% of said plurality of test genes are cell-cycle genes (e.g., genes in any of Tables 1-11 or Panels A-H, J, or K; “sub-panels” of Panel F in Tables A′ to E′); and (4)(a) correlating an increased level of expression of the plurality of test genes to a poor prognosis and/or an increased likelihood of response to the particular treatment regimen (e.g., a treatment regimen comprising chemotherapy) or (b) correlating no increase in the overall expression of the test genes to a good prognosis and/or no increased likelihood of response to the treatment. In some embodiments, instead of (optionally in addition to) the correlating step(s), the method comprises (4)(a) concluding that the patient has a poor prognosis and/or an increased likelihood of response to the particular treatment regimen based at least in part on increased expression of said plurality of test genes or (b) concluding that the patient has a good prognosis and/or no increased likelihood of response to the particular treatment regimen based at least in part on no increased expression of said plurality of test genes; and/or (4)(a) communicating that the patient has a poor prognosis and/or an increased likelihood of response to the particular treatment regimen based at least in part on increased expression of said plurality of test genes or (b) communicating that the patient has a good prognosis and/or no increased likelihood of response to the particular treatment regimen based at least in part on no increased expression of said plurality of test genes. In some embodiments the test genes are weighted such that the cell-cycle genes are weighted to contribute at least 50%, at least 55%, at least 60%, at least 65%, at least 75%, at least 80%, at least 85%, at least 90%, at least 95%, at least 99% or 100% of the test value. In some embodiments 20%, 25%, 30%, 35%, 40%, 45%, 50%, 55%, 60%, 65%, 75%, 80%, 85%, 90%, 95%, or at least 99% or 100% of the plurality of test genes are cell-cycle genes. Unless otherwise indicated, “obtaining a sample” herein means “providing or obtaining”

Accordingly, in some embodiments the method comprises: (1) obtaining a tumor sample from a patient identified as having lung cancer; (2) determining the expression of a panel of genes in the tumor sample including at least 2, 4, 6, 8 or 10 cell-cycle genes (e.g., genes in any of Tables 1-11 or Panels A-H, J, or K; “sub-panels” of Panel F in Tables A′ to E′); and (3) providing a test value by (a) weighting the determined expression of each of a plurality of test genes selected from said panel of genes with a predefined coefficient, and (b) combining the weighted expression to provide said test value, wherein the cell-cycle genes are weighted to contribute at least 20%, 50%, at least 75% or at least 90% of the test value; and (4)(a) correlating an increased level of expression of the plurality of test genes to a poor prognosis and/or an increased likelihood of response to the particular treatment regimen (e.g., a treatment regimen comprising chemotherapy) or (b) correlating no increased level of expression of the plurality of test genes to a good prognosis and/or a no increased likelihood of response to the particular treatment. In some embodiments, instead of (optionally in addition to) the correlating step(s), the method comprises (4)(a) concluding that the patient has a poor prognosis and/or an increased likelihood of response to the particular treatment regimen based at least in part on increased expression of said plurality of test genes or (b) concluding that the patient has a good prognosis and/or no increased likelihood of response to the particular treatment regimen based at least in part on no increased expression of said plurality of test genes; and/or (4)(a) communicating that the patient has a poor prognosis and/or an increased likelihood of response to the particular treatment regimen based at least in part on increased expression of said plurality of test genes or (b) communicating that the patient has a good prognosis and/or no increased likelihood of response to the particular treatment regimen based at least in part on no increased expression of said plurality of test genes.

In each embodiment described herein involving CCP gene expression levels, the present invention encompasses a further, related embodiment involving a test value or score (e.g., CCP score, etc.) derived from, incorporating, and/or, at least to some degree, reflecting such expression levels. In other words, the bare CCP gene expressions data or levels need not be used in the various methods, systems, etc. of the invention; a test value or score derived from such numbers or lengths may be used. Typically, such test value will be compared to a reference value (as described at length in this document) and the method will end by correlating a high test value (or a test value derived from, incorporating, and/or, at least to some degree, reflecting high CCP gene expression) to a poor prognosis. The invention encompasses, mutatis mutandis, corresponding embodiments where the test value or score is used to determine the patient's prognosis, the patient's likelihood of response to a particular treatment regimen, the patient's or patient's sample's likelihood of having a breast cancer recurrence, etc.

The invention generally comprises determining the status of a panel of genes comprising at least two CCGs, in tissue or cell sample, particularly a tumor sample, from a patient. As used herein, “determining the status” of a gene (or panel of genes) refers to determining the presence, absence, or extent/level of some physical, chemical, or genetic characteristic of the gene or its expression product(s). Such characteristics include, but are not limited to, expression levels, activity levels, mutations, copy number, methylation status, etc.

In the context of CCGs as used to determine likelihood of response to a particular treatment regimen (e.g., a treatment regimen comprising chemotherapy), particularly useful characteristics include expression levels (e.g., mRNA, cDNA or protein levels) and activity levels. Characteristics may be assayed directly (e.g., by assaying a CCG's expression level) or determined indirectly (e.g., assaying the level of a gene or genes whose expression level is correlated to the expression level of the CCG).

“Abnormal status” means a marker's status in a particular sample differs from the status generally found in average samples (e.g., healthy samples, average diseased samples). Examples include mutated, elevated, decreased, present, absent, etc. An “elevated status” means that one or more of the above characteristics (e.g., expression or mRNA level) is higher than normal levels. Generally this means an increase in the characteristic (e.g., expression or mRNA level) as compared to an index value as discussed below. Conversely a “low status” means that one or more of the above characteristics (e.g., gene expression or mRNA level) is lower than normal levels. Generally this means a decrease in the characteristic (e.g., expression) as compared to an index value as discussed below. In this context, a “negative status” generally means the characteristic is absent or undetectable or, in the case of sequence analysis, there is a deleterious sequence variant (including full or partial gene deletion).

Gene expression can be determined either at the RNA level (i.e., mRNA or noncoding RNA (ncRNA)) (e.g., miRNA, tRNA, rRNA, snoRNA, siRNA and piRNA) or at the protein level. Measuring gene expression at the mRNA level includes measuring levels of cDNA corresponding to mRNA. Levels of proteins in a tumor sample can be determined by any known technique in the art, e.g., HPLC, mass spectrometry, or using antibodies specific to selected proteins (e.g., IHC, ELISA, etc.).

In some embodiments, the amount of RNA transcribed from the panel of genes including test genes is measured in the tumor sample. In addition, the amount of RNA of one or more housekeeping genes in the tumor sample is also measured, and used to normalize or calibrate the expression of the test genes. The terms “normalizing genes” and “housekeeping genes” are defined herein below.

In any embodiment of the invention involving a “plurality of test genes,” the plurality of test genes may include at least 2, 3 or 4 cell-cycle genes, which constitute at least 50%, 75% or 80% of the plurality of test genes, and preferably 100% of the plurality of test genes. In other such embodiments, the plurality of test genes includes at least 5, 6, 7, or at least 8 cell-cycle genes, which constitute at least 20%, 25%, 30%, 40%, 50%, 60%, 70%, 75%, 80% or 90% of the plurality of test genes, and preferably 100% of the plurality of test genes. As will be clear from the context of this document, a panel of genes is a plurality of genes. In some embodiments these genes are assayed together in one or more samples from a patient.

In some embodiments, the plurality of test genes includes at least 8, 10, 12, 15, 20, 25 or 30 cell-cycle genes, which constitute at least 20%, 25%, 30%, 40%, 50%, 60%, 70%, 75%, 80% or 90% of the plurality of test genes, and preferably 100% of the plurality of test genes.

As will be apparent to a skilled artisan apprised of the present invention and the disclosure herein, “tumor sample” means any biological sample containing one or more tumor cells, or one or more tumor-derived DNA, RNA or protein, and obtained from a cancer patient. For example, a tissue sample obtained from a tumor tissue of a cancer patient is a useful tumor sample in the present invention. The tissue sample can be an FFPE sample, or fresh frozen sample, and preferably contain largely tumor cells. A single malignant cell from a cancer patient's tumor is also a useful tumor sample. Such a malignant cell can be obtained directly from the patient's tumor, or purified from the patient's bodily fluid (e.g., blood, urine). Thus, a bodily fluid such as blood, urine, sputum and saliva containing one or tumor cells, or tumor-derived RNA or proteins, can also be useful as a tumor sample for purposes of practicing the present invention. In some embodiments, the patient having a cancer (e.g., lung cancer) has been diagnosed with that cancer.

Those skilled in the art are familiar with various techniques for determining the status of a gene or protein in a tissue or cell sample including, but not limited to, microarray analysis (e.g., for assaying mRNA or microRNA expression, copy number, etc.), quantitative real-time PCR™ (“qRT-PCR™”, e.g., TaqMan™), immunoanalysis (e.g., ELISA, immunohistochemistry), sequencing (e.g., quantitative sequencing), etc. The activity level of a polypeptide encoded by a gene may be used in much the same way as the expression level of the gene or polypeptide. Often higher activity levels indicate higher expression levels and while lower activity levels indicate lower expression levels. Thus, in some embodiments, the invention provides any of the methods discussed above, wherein the activity level of a polypeptide encoded by the CCG is determined rather than or in addition to the expression level of the CCG. Those skilled in the art are familiar with techniques for measuring the activity of various such proteins, including those encoded by the genes listed in Exemplary CCGs are listed in Tables 1, 2, 3, 5, 6, 7, 8, 9, 10 & 11. The methods of the invention may be practiced independent of the particular technique used.

In preferred embodiments, the expression of one or more normalizing (often called “housekeeping”) genes is also obtained for use in normalizing the expression of test genes. As used herein, “normalizing genes” referred to the genes whose expression is used to calibrate or normalize the measured expression of the gene of interest (e.g., test genes). Importantly, the expression of normalizing genes should be independent of cancer outcome/prognosis, and the expression of the normalizing genes is very similar among all the tumor samples. The normalization ensures accurate comparison of expression of a test gene between different samples. For this purpose, housekeeping genes known in the art can be used. Housekeeping genes are well known in the art, with examples including, but are not limited to, GUSB (glucuronidase, beta), HMBS (hydroxymethylbilane synthase), SDHA (succinate dehydrogenase complex, subunit A, flavoprotein), UBC (ubiquitin C) and YWHAZ (tyrosine 3-monooxygenase/tryptophan 5-monooxygenase activation protein, zeta polypeptide). One or more housekeeping genes can be used. Preferably, at least 2, 3, 4, 5, 6, 7, 8, 9, 10 or 15 housekeeping genes are used to provide a combined normalizing gene set. The amount of gene expression of such normalizing genes can be averaged, combined together by straight additions or by a defined algorithm. Some examples of particularly useful housekeeper genes for use in the methods and compositions of the invention include those listed in Table A below.

TABLE A

| | | Applied | |
|---|---|---|---|
| Gene | Entrez | Biosystems | RefSeq Accession |
| Symbol | GeneID | Assay ID | Nos. |
| CLTC* | 1213 | Hs00191535_m1 | NM_004859.3 |
| GUSB | 2990 | Hs99999908_m1 | NM_000181.2 |
| HMBS | 3145 | Hs00609297_m1 | NM_000190.3 |
| MMADHC* | 27249 | Hs00739517_g1 | NM_015702.2 |
| MRFAP1* | 93621 | Hs00738144_g1 | NM_033296.1 |
| PPP2CA* | 5515 | Hs00427259_m1 | NM_002715.2 |
| PSMA1* | 5682 | Hs00267631_m1 | |
| PSMC1* | 5700 | Hs02386942_g1 | NM_002802.2 |
| RPL13A* | 23521 | Hs03043885_g1 | NM_012423.2 |
| RPL37* | 6167 | Hs02340038_g1 | NM_000997.4 |
| RPL38* | 6169 | Hs00605263_g1 | NM_000999.3 |
| RPL4* | 6124 | Hs03044647_g1 | NM_000968.2 |
| RPL8* | 6132 | Hs00361285_g1 | NM_033301.1; |
| | | | NM_000973.3 |
| RPS29* | 6235 | Hs03004310_g1 | NM_001030001.1; |
| | | | NM_001032.3 |
| SDHA | 6389 | Hs00188166_m1 | NM_004168.2 |
| SLC25A3* | 6515 | Hs00358082_m1 | NM_213611.1; |
| | | | NM_002635.2; |
| | | | NM_005888.2 |
| TXNL1* | 9352 | Hs00355488_m1 | NR_024546.1; |
| | | | NM_004786.2 |
| UBA52* | 7311 | Hs03004332_g1 | NM_001033930.1; |
| | | | NM_003333.3 |
| UBC | 7316 | Hs00824723_m1 | NM_021009.4 |
| YWHAZ | 7534 | Hs00237047_m1 | NM_003406.3 |

*Subset of housekeeping genes used in normalizing CCGs and generating the CCP Score in Example 1.

In the case of measuring RNA levels for the genes, one convenient and sensitive approach is real-time quantitative PCR™ (qPCR) assay, following a reverse transcription reaction. Typically, a cycle threshold (Cₜ) is determined for each test gene and each normalizing gene, i.e., the number of cycles at which the fluorescence from a qPCR reaction above background is detectable.

The overall expression of the one or more normalizing genes can be represented by a “normalizing value” which can be generated by combining the expression of all normalizing genes, either weighted equally (straight addition or averaging) or by different predefined coefficients. For example, in a simplest manner, the normalizing value C_{tH} can be the cycle threshold (Cₜ) of one single normalizing gene, or an average of the Cₜ values of 2 or more, preferably 10 or more, or 15 or more normalizing genes, in which case, the predefined coefficient is 1/N, where N is the total number of normalizing genes used. Thus, C_{tH}=(C_{tH1}+C_{tH2}+C_{tHn})/N. As will be apparent to skilled artisans, depending on the normalizing genes used, and the weight desired to be given to each normalizing gene, any coefficients (from 0/N to N/N) can be given to the normalizing genes in weighting the expression of such normalizing genes. That is, C_{tH}=×C_{tH1}+yC_{tH2}+ . . . zC_{tHn}, wherein x+y+ . . . +z=1.

As discussed above, the methods of the invention generally involve determining the level of expression of a panel of CCGs. With modern high-throughput techniques, it is often possible to determine the expression level of tens, hundreds or thousands of genes. Indeed, it is possible to determine the level of expression of the entire transcriptome (i.e., each transcribed sequence in the genome). Once such a global assay has been performed, one may then informatically analyze one or more subsets of transcripts (i.e., panels or, as often used herein, pluralities of test genes). After measuring the expression of hundreds or thousands of transcripts in a sample, for example, one may analyze (e.g., informatically) the expression of a panel or plurality of test genes comprising primarily CCGs according to the present invention by combining the expression level values of the individual test genes to obtain a test value.

As will be apparent to a skilled artisan, the test value provided in the present invention can represent the overall expression level of the plurality of test genes composed substantially of (or weighted to be represented substantially by) cell-cycle genes. In one embodiment, to provide a test value in the methods of the invention, the normalized expression for a test gene can be obtained by normalizing the measured Cₜ for the test gene against the C_{tH}, i.e., ΔCₜ₁=(Cₜ₁−C_{tH}). Thus, the test value incorporating the overall expression of the plurality of test genes can be provided by combining the normalized expression of all test genes, either by straight addition or averaging (i.e., weighted equally) or by a different predefined coefficient. For example, the simplest approach is averaging the normalized expression of all test genes: test value=(ΔCₜ₁+ΔCₜ₂+ . . . +ΔCₜₙ)/n. As will be apparent to skilled artisans, depending on the test genes used, different weight can also be given to different test genes in the present invention. In each case where this document discloses using the expression of a plurality of genes (e.g., “determining [in a tumor sample from the patient] the expression of a plurality of test genes” or “correlating increased expression of said plurality of test genes to an increased likelihood of response”), this includes in some embodiments using a test value incorporating, representing or corresponding to the overall expression of this plurality of genes (e.g., “determining [in a tumor sample from the patient] a test value representing the expression of a plurality of test genes” or “correlating an increased test value [or a test value above some reference value] representing the expression of said plurality of test genes to an increased likelihood of response”).

It has been determined that, once the CCP phenomenon reported herein is appreciated, the choice of individual CCGs for a test panel can, in some embodiments, be somewhat arbitrary. In other words, many CCGs have been found to be very good surrogates for each other. Thus any CCG (or panel of CCGs) can be used in the various embodiments of the invention. In other embodiments of the invention, optimized CCGs are used. One way of assessing whether particular CCGs will serve well in the methods and compositions of the invention is by assessing their correlation with the mean expression of CCGs (e.g., all known CCGs, a specific set of CCGs, etc.). Those CCGs that correlate particularly well with the mean are expected to perform well in assays of the invention, e.g., because these will reduce noise in the assay.

126 CCGs and 47 housekeeping genes had their expression compared to the CCG and housekeeping mean in order to determine preferred genes for use in some embodiments of the invention. Rankings of select CCGs according to their correlation with the mean CCG expression as well as their ranking according to predictive value are given in Tables 2, 3, 5, 6, 7, 12, 13, 14, 15, 16, 17, 18 & 19.

Some CCGs do not correlate well with the mean. In some embodiments of the present invention, such genes may be grouped, assayed, analyzed, etc. separately from those that correlate well. This is especially useful if these non-correlated genes are independently associated with the clinical feature of interest (e.g., prognosis, therapy response, etc.). Thus, in some embodiments of the invention, non-correlated genes are analyzed together with correlated genes. In some embodiments, a CCG is non-correlated if its correlation to the CCG mean is less than 0.5, 0.4, 0.3, 0.2, 0.10, 0.09, 0.08, 0.07, 0.06, 0.05, 0.04, 0.03, 0.02, 0.01 or less.

Assays of 126 CCGs and 47 HK (housekeeping) genes were run against 96 commercially obtained, anonymous tumor FFPE samples without outcome or other clinical data. The working hypothesis was that the assays would measure with varying degrees of accuracy the same underlying phenomenon (cell cycle proliferation within the tumor for the CCGs, and sample concentration for the HK genes). Assays were ranked by the Pearson's correlation coefficient between the individual gene and the mean of all the candidate genes, that being the best available estimate of biological activity. Rankings for these 126 CCGs according to their correlation to the overall CCG mean are reported in Table 2.

TABLE 2

| Gene # | Gene Symbol | Correl. w/ Mean |
|---|---|---|
| 1 | TPX2 | 0.931 |
| 2 | CCNB2 | 0.9287 |
| 3 | KIF4A | 0.9163 |
| 4 | KIF2C | 0.9147 |
| 5 | BIRC5 | 0.9077 |
| 6 | BIRC5 | 0.9077 |
| 7 | RACGAP1 | 0.9073 |
| 8 | CDC2 | 0.906 |
| 9 | PRC1 | 0.9053 |
| 10 | DLGAP5/ | 0.9033 |
| | DLG7 | |
| 11 | CEP55 | 0.903 |
| 12 | CCNB1 | 0.9 |
| 13 | TOP2A | 0.8967 |
| 14 | CDC20 | 0.8953 |
| 15 | KIF20A | 0.8927 |
| 16 | BUB1B | 0.8927 |
| 17 | CDKN3 | 0.8887 |
| 18 | NUSAP1 | 0.8873 |
| 19 | CCNA2 | 0.8853 |
| 20 | KIF11 | 0.8723 |
| 21 | CDCA8 | 0.8713 |
| 22 | NCAPG | 0.8707 |
| 23 | ASPM | 0.8703 |
| 24 | FOXM1 | 0.87 |
| 25 | NEK2 | 0.869 |
| 26 | ZWINT | 0.8683 |
| 27 | PTTG1 | 0.8647 |
| 28 | RRM2 | 0.8557 |
| 29 | TTK | 0.8483 |
| 30 | TRIP13 | 0.841 |
| 31 | GINS1 | 0.841 |
| 32 | CENPF | 0.8397 |
| 33 | HMMR | 0.8367 |
| 34 | NCAPH | 0.8353 |
| 35 | NDC80 | 0.8313 |
| 36 | KIF15 | 0.8307 |
| 37 | CENPE | 0.8287 |
| 38 | TYMS | 0.8283 |
| 39 | KIAA0101 | 0.8203 |
| 40 | FANCI | 0.813 |
| 41 | RAD51AP1 | 0.8107 |
| 42 | CKS2 | 0.81 |
| 43 | MCM2 | 0.8063 |
| 44 | PBK | 0.805 |
| 45 | ESPL1 | 0.805 |
| 46 | MKI67 | 0.7993 |
| 47 | SPAG5 | 0.7993 |
| 48 | MCM10 | 0.7963 |
| 49 | MCM6 | 0.7957 |
| 50 | OIP5 | 0.7943 |
| 51 | CDC45L | 0.7937 |
| 52 | KIF23 | 0.7927 |
| 53 | EZH2 | 0.789 |
| 54 | SPC25 | 0.7887 |
| 55 | STIL | 0.7843 |
| 56 | CENPN | 0.783 |
| 57 | GTSE1 | 0.7793 |
| 58 | RAD51 | 0.779 |
| 59 | CDCA3 | 0.7783 |
| 60 | TACC3 | 0.778 |
| 61 | PLK4 | 0.7753 |
| 62 | ASF1B | 0.7733 |
| 63 | DTL | 0.769 |
| 64 | CHEK1 | 0.7673 |
| 65 | NCAPG2 | 0.7667 |
| 66 | PLK1 | 0.7657 |
| 67 | TIMELESS | 0.762 |
| 68 | E2F8 | 0.7587 |
| 69 | EXO1 | 0.758 |
| 70 | ECT2 | 0.744 |
| 71 | STMN1 | 0.737 |
| 72 | STMN1 | 0.737 |
| 73 | RFC4 | 0.737 |
| 74 | CDC6 | 0.7363 |
| 75 | CENPM | 0.7267 |
| 76 | MYBL2 | 0.725 |
| 77 | SHCBP1 | 0.723 |
| 78 | ATAD2 | 0.723 |
| 79 | KIFC1 | 0.7183 |
| 80 | DBF4 | 0.718 |
| 81 | CKS1B | 0.712 |
| 82 | PCNA | 0.7103 |
| 83 | FBXO5 | 0.7053 |
| 84 | C12orf48 | 0.7027 |
| 85 | TK1 | 0.7017 |
| 86 | BLM | 0.701 |
| 87 | KIF18A | 0.6987 |
| 88 | DONSON | 0.688 |
| 89 | MCM4 | 0.686 |
| 90 | RAD54B | 0.679 |
| 91 | RNASEH2A | 0.6733 |
| 92 | TUBA1C | 0.6697 |
| 93 | C18orf24 | 0.6697 |
| 94 | SMC2 | 0.6697 |
| 95 | CENPI | 0.6697 |
| 96 | GMPS | 0.6683 |
| 97 | DDX39 | 0.6673 |
| 98 | POLE2 | 0.6583 |
| 99 | APOBEC3B | 0.6513 |
| 100 | RFC2 | 0.648 |
| 101 | PSMA7 | 0.6473 |
| 102 | MPHOSPH1/ | 0.6457 |
| | kif20b | |
| 103 | CDT1 | 0.645 |
| 104 | H2AFX | 0.6387 |
| 105 | ORC6L | 0.634 |
| 106 | Clorf135 | 0.6333 |
| 107 | PSRC1 | 0.633 |
| 108 | VRK1 | 0.6323 |
| 109 | CKAP2 | 0.6307 |
| 110 | CCDC99 | 0.6303 |
| 111 | CCNE1 | 0.6283 |
| 112 | LMNB2 | 0.625 |
| 113 | GPSM2 | 0.625 |
| 114 | PAICS | 0.6243 |
| 115 | MCAM | 0.6227 |
| 116 | DSN1 | 0.622 |
| 117 | NCAPD2 | 0.6213 |
| 118 | RAD54L | 0.6213 |
| 119 | PDSS1 | 0.6203 |
| 120 | HN1 | 0.62 |
| 121 | C21orf45 | 0.6193 |
| 122 | CTSL2 | 0.619 |
| 123 | CTPS | 0.6183 |
| 124 | MCM7 | 0.618 |
| 125 | ZWILCH | 0.618 |
| 126 | RFC5 | 0.6177 |

After excluding CCGs with low average expression, assays that produced sample failures, CCGs with correlations less than 0.58, and HK genes with correlations less than 0.95, a subset of 56 CCGs (Panel G) and 36 HK candidate genes were left. Correlation coefficients were recalculated on these subsets, with the rankings shown in Tables 3 and 4, respectively.

TABLE 3
(“Panel G”)

| | Gene | Correl. w/ |
|---|---|---|
| Gene # | Symbol | CCG mean |
| 1 | FOXM1 | 0.908 |
| 2 | CDC20 | 0.907 |
| 3 | CDKN3 | 0.9 |
| 4 | CDC2 | 0.899 |
| 5 | KIF11 | 0.898 |
| 6 | KIAA0101 | 0.89 |
| 7 | NUSAP1 | 0.887 |
| 8 | CENPF | 0.882 |
| 9 | ASPM | 0.879 |
| 10 | BUB1B | 0.879 |
| 11 | RRM2 | 0.876 |
| 12 | DLGAP5 | 0.875 |
| 13 | BIRC5 | 0.864 |
| 14 | KIF20A | 0.86 |
| 15 | PLK1 | 0.86 |
| 16 | TOP2A | 0.851 |
| 17 | TK1 | 0.837 |
| 18 | PBK | 0.831 |
| 19 | ASF1B | 0.827 |
| 20 | C18orf24 | 0.817 |
| 21 | RAD54L | 0.816 |
| 22 | PTTG1 | 0.814 |
| 23 | KIF4A | 0.814 |
| 24 | CDCA3 | 0.811 |
| 25 | MCM10 | 0.802 |
| 26 | PRC1 | 0.79 |
| 27 | DTL | 0.788 |
| 28 | CEP55 | 0.787 |
| 29 | RAD51 | 0.783 |
| 30 | CENPM | 0.781 |
| 31 | CDCA8 | 0.774 |
| 32 | OIP5 | 0.773 |
| 33 | SHCBP1 | 0.762 |
| 34 | ORC6L | 0.736 |
| 35 | CCNB1 | 0.727 |
| 36 | CHEK1 | 0.723 |
| 37 | TACC3 | 0.722 |
| 38 | MCM4 | 0.703 |
| 39 | FANCI | 0.702 |
| 40 | KIF15 | 0.701 |
| 41 | PLK4 | 0.688 |
| 42 | APOBEC3B | 0.67 |
| 43 | NCAPG | 0.667 |
| 44 | TRIP13 | 0.653 |
| 45 | KIF23 | 0.652 |
| 46 | NCAPH | 0.649 |
| 47 | TYMS | 0.648 |
| 48 | GINS1 | 0.639 |
| 49 | STMN1 | 0.63 |
| 50 | ZWINT | 0.621 |
| 51 | BLM | 0.62 |
| 52 | TTK | 0.62 |
| 53 | CDC6 | 0.619 |
| 54 | KIF2C | 0.596 |
| 55 | RAD51AP1 | 0.567 |
| 56 | NCAPG2 | 0.535 |

TABLE 4

| | | Correlation |
|---|---|---|
| | Gene | with HK |
| Gene # | Symbol | Mean |
| 1 | RPL38 | 0.989 |
| 2 | UBA52 | 0.986 |
| 3 | PSMC1 | 0.985 |
| 4 | RPL4 | 0.984 |
| 5 | RPL37 | 0.983 |
| 6 | RPS29 | 0.983 |
| 7 | SLC25A3 | 0.982 |
| 8 | CLTC | 0.981 |
| 9 | TXNL1 | 0.98 |
| 10 | PSMA1 | 0.98 |
| 11 | RPL8 | 0.98 |
| 12 | MMADHC | 0.979 |
| 13 | RPL13A; | 0.979 |
| | LOC728658 | |
| 14 | PPP2CA | 0.978 |
| 15 | MRFAP1 | 0.978 |

The CCGs in Panel F were likewise ranked according to correlation to the CCG mean as shown in Table 5 below.

TABLE 5

| | Gene | Correl. w/ |
|---|---|---|
| Gene # | Symbol | CCG mean |
| 1 | DLGAP5 | 0.931 |
| 2 | ASPM | 0.931 |
| 3 | KIF11 | 0.926 |
| 4 | BIRC5 | 0.916 |
| 5 | CDCA8 | 0.902 |
| 6 | CDC20 | 0.9 |
| 7 | MCM10 | 0.899 |
| 8 | PRC1 | 0.895 |
| 9 | BUB1B | 0.892 |
| 10 | FOXM1 | 0.889 |
| 11 | NUSAP1 | 0.888 |
| 12 | C18orf24 | 0.885 |
| 13 | PLK1 | 0.879 |
| 14 | CDKN3 | 0.874 |
| 15 | RRM2 | 0.871 |
| 16 | RAD51 | 0.864 |
| 17 | CEP55 | 0.862 |
| 18 | ORC6L | 0.86 |
| 19 | RAD54L | 0.86 |
| 20 | CDC2 | 0.858 |
| 21 | CENPF | 0.855 |
| 22 | TOP2A | 0.852 |
| 23 | KIF20A | 0.851 |
| 24 | KIAA0101 | 0.839 |
| 25 | CDCA3 | 0.835 |
| 26 | ASF1B | 0.797 |
| 27 | CENPM | 0.786 |
| 28 | TK1 | 0.783 |
| 29 | PBK | 0.775 |
| 30 | PTTG1 | 0.751 |
| 31 | DTL | 0.737 |

When choosing specific CCGs for inclusion in any embodiment of the invention, the individual predictive power of each gene may be used to rank them in importance. The inventors have determined that the CCGs in Panel C can be ranked as shown in Table 6 below according to the predictive power of each individual gene. The CCGs in Panel F can be similarly ranked as shown in Table 7 below.

TABLE 6

| Gene # | Gene | p-value |
|---|---|---|
| 1 | NUSAP1 | 2.8E−07 |
| 2 | DLG7 | 5.9E−07 |
| 3 | CDC2 | 6.0E−07 |
| 4 | FOXM1 | 1.1E−06 |
| 5 | MYBL2 | 1.1E−06 |
| 6 | CDCA8 | 3.3E−06 |
| 7 | CDC20 | 3.8E−06 |
| 8 | RRM2 | 7.2E−06 |
| 9 | PTTG1 | 1.8E−05 |
| 10 | CCNB2 | 5.2E−05 |
| 11 | HMMR | 5.2E−05 |
| 12 | BUB1 | 8.3E−05 |
| 13 | PBK | 1.2E−04 |
| 14 | TTK | 3.2E−04 |
| 15 | CDC45L | 7.7E−04 |
| 16 | PRC1 | 1.2E−03 |
| 17 | DTL | 1.4E−03 |
| 18 | CCNB1 | 1.5E−03 |
| 19 | TPX2 | 1.9E−03 |
| 20 | ZWINT | 9.3E−03 |
| 21 | KIF23 | 1.1E−02 |
| 22 | TRIP13 | 1.7E−02 |
| 23 | KPNA2 | 2.0E−02 |
| 24 | UBE2C | 2.2E−02 |
| 25 | MELK | 2.5E−02 |
| 26 | CENPA | 2.9E−02 |
| 27 | CKS2 | 5.7E−02 |
| 28 | MAD2L1 | 1.7E−01 |
| 29 | UBE2S | 2.0E−01 |
| 30 | AURKA | 4.8E−01 |
| 31 | TIMELESS | 4.8E−01 |

TABLE 7

| Gene # | Gene Symbol | p-value |
|---|---|---|
| 1 | MCM10 | 8.60E−10 |
| 2 | ASPM | 2.30E−09 |
| 3 | DLGAP5 | 1.20E−08 |
| 4 | CENPF | 1.40E−08 |
| 5 | CDC20 | 2.10E−08 |
| 6 | FOXM1 | 3.40E−07 |
| 7 | TOP2A | 4.30E−07 |
| 8 | NUSAP1 | 4.70E−07 |
| 9 | CDKN3 | 5.50E−07 |
| 10 | KIF11 | 6.30E−06 |
| 11 | KIF20A | 6.50E−06 |
| 12 | BUB1B | 1.10E−05 |
| 13 | RAD54L | 1.40E−05 |
| 14 | CEP55 | 2.60E−05 |
| 15 | CDCA8 | 3.10E−05 |
| 16 | TK1 | 3.30E−05 |
| 17 | DTL | 3.60E−05 |
| 18 | PRC1 | 3.90E−05 |
| 19 | PTTG1 | 4.10E−05 |
| 20 | CDC2 | 0.00013 |
| 21 | ORC6L | 0.00017 |
| 22 | PLK1 | 0.0005 |
| 23 | C18orf24 | 0.0011 |
| 24 | BIRC5 | 0.00118 |
| 25 | RRM2 | 0.00255 |
| 26 | CENPM | 0.0027 |
| 27 | RAD51 | 0.0028 |
| 28 | KIAA0101 | 0.00348 |
| 29 | CDCA3 | 0.00863 |
| 30 | PBK | 0.00923 |
| 31 | ASF1B | 0.00936 |

Thus, in some embodiments of each of the various aspects of the invention the plurality of test genes comprises the top 2, 3, 4, 5, 6, 7, 8, 9, 10, 11, 12, 13, 14, 15, 20, 25, 30, 35, 40 or more CCGs listed in Table 2, 3, 5, 6, 7, 12, 13, 14, 15, 16, 17, 18 or 19. In some embodiments the plurality of test genes comprises at least some number of CCGs (e.g., at least 3, 4, 5, 6, 7, 8, 9, 10, 15, 20, 25, 30, 35, 40, 45, 50 or more CCGs) and this plurality of CCGs comprises at least 1, 2, 3, 4, 5, 6, 7, 8, 9, 10, 15, or 20 of the following genes: ASPM, BIRC5, BUB1B, CCNB2, CDC2, CDC20, CDCA8, CDKN3, CENPF, DLGAP5, FOX111, KIAA0101, KIF 11, KIF2C, KIF4A, MCM10, NUSAP1, PRC1, RACGAP1, and TPX2. In some embodiments the plurality of test genes comprises at least some number of CCGs (e.g., at least 3, 4, 5, 6, 7, 8, 9, 10, 15, 20, 25, 30, 35, 40, 45, 50 or more CCGs) and this plurality of CCGs comprises at least 1, 2, 3, 4, 5, 6, 7, 8, 9, 10, 15, or 20 of the following genes: TPX2, CCNB2, KIF4A, KIF2C, BIRC5, RACGAP1, CDC2, PRC1, DLGAP5/DLG7, CEP55, CCNB1, TOP2A, CDC20, KIF20A, BUB1B, CDKN3, NUSAP1, CCNA2, KIF11, and CDCA8. In some embodiments the plurality of test genes comprises at least some number of CCGs (e.g., at least 3, 4, 5, 6, 7, 8, 9, 10, 15, 20, 25, 30, 35, 40, 45, 50 or more CCGs) and this plurality of CCGs comprises any one, two, three, four, five, six, seven, eight, nine, or ten or all of gene numbers 1 & 2, 1 to 3, 1 to 4, 1 to 5, 1 to 6, 1 to 7, 1 to 8, 1 to 9, or 1 to 10 of any of Table 2, 3, 5, 6, 7, 12, 13, 14, 15, 16, 17, 18 or 19. In some embodiments the plurality of test genes comprises at least some number of CCGs (e.g., at least 3, 4, 5, 6, 7, 8, 9, 10, 15, 20, 25, 30, 35, 40, 45, 50 or more CCGs) and this plurality of CCGs comprises any one, two, three, four, five, six, seven, eight, or nine or all of gene numbers 2 & 3, 2 to 4, 2 to 5, 2 to 6, 2 to 7, 2 to 8, 2 to 9, or 2 to 10 of any of Table 2, 3, 5, 6, 7, 12, 13, 14, 15, 16, 17, 18 or 19. In some embodiments the plurality of test genes comprises at least some number of CCGs (e.g., at least 3, 4, 5, 6, 7, 8, 9, 10, 15, 20, 25, 30, 35, 40, 45, 50 or more CCGs) and this plurality of CCGs comprises any one, two, three, four, five, six, seven, or eight or all of gene numbers 3 & 4, 3 to 5, 3 to 6, 3 to 7, 3 to 8, 3 to 9, or 3 to 10 of any of Table 2, 3, 5, 6, 7, 12, 13, 14, 15, 16, 17, 18 or 19. In some embodiments the plurality of test genes comprises at least some number of CCGs (e.g., at least 3, 4, 5, 6, 7, 8, 9, 10, 15, 20, 25, 30, 35, 40, 45, 50 or more CCGs) and this plurality of CCGs comprises any one, two, three, four, five, six, or seven or all of gene numbers 4 & 5, 4 to 6, 4 to 7, 4 to 8, 4 to 9, or 4 to 10 of any of Table 2, 3, 5, 6, 7, 12, 13, 14, 15, 16, 17, 18 or 19. In some embodiments the plurality of test genes comprises at least some number of CCGs (e.g., at least 3, 4, 5, 6, 7, 8, 9, 10, 15, 20, 25, 30, 35, 40, 45, 50 or more CCGs) and this plurality of CCGs comprises any one, two, three, four, five, six, seven, eight, nine, 10, 11, 12, 13, 14, or 15 or all of gene numbers 1 & 2, 1 to 3, 1 to 4, 1 to 5, 1 to 6, 1 to 7, 1 to 8, 1 to 9, 1 to 10, 1 to 11, 1 to 12, 1 to 13, 1 to 14, or 1 to 15 of any of Table 2, 3, 5, 6, 7, 12, 13, 14, 15, 16, 17, 18 or 19.

In some embodiments the plurality of test genes comprises at least some number of CCGs (e.g., at least 3, 4, 5, 6, 7, 8, 9, 10, 15, 20, 25, 30, 35, 40, 45, 50 or more CCGs) and this plurality of CCGs comprises gene numbers 1 & 2; 1 & 2-3; 1 & 3-4; 1 & 4-5; 1 & 5-6; 1 & 6-7; 1 & 7-8; 1 & 8-9; 1 & 9 & 10; 1 & 10 & 11; 1 & 3; 1 & 2-4; 1 & 3-5; 1 & 4-6; 1 & 5-7; 1 & 6-8; 1 & 7-9; 1 & 8-10; 1 & 9 & 11; 1 & 4; 1 & 2-5; 1 & 3-6; 1 & 4-7; 1 & 5-8; 1 & 6-9; 1 & 7-10; 1 & 8-11; 1 & 5; 1 & 2-6; 1 & 3-7; 1 & 4-8; 1 & 5-9; 1 & 6-10; 1 & 7-11; 1 & 6; 1 & 2-7; 1 & 3-8; 1 & 4-9; 1 & 5-10; 1 & 6-11; 1 & 7; 1 & 2-8; 1 & 3-9; 1 & 4-10; 1 & 5-11; 1 & 8; 1 & 2-9; 1 & 3-10; 1 & 4-11; 1 & 9; 1 & 2-10; 1 & 3-11; 1 & 10; 1 & 2-11; 1 & 11; 2 & 3; 2 & 3-4; 2 & 4-5; 2 & 5-6; 2 & 6-7; 2 & 7-8; 2 & 8-9; 2 & 9 & 10; 2 & 10 & 11; 2 & 4; 2 & 3-5; 2 & 4-6; 2 & 5-7; 2 & 6-8; 2 & 7-9; 2 & 8-10; 2 & 9 & 11; 2 & 5; 2 & 3-6; 2 & 4-7; 2 & 5-8; 2 & 6-9; 2 & 7-10; 2 & 8-11; 2 & 6; 2 & 3-7; 2 & 4-8; 2 & 5-9; 2 & 6-10; 2 & 7-11; 2 & 7; 2 & 3-8; 2 & 4-9; 2 & 5-10; 2 & 6-11; 2 & 8; 2 & 3-9; 2 & 4-10; 2 & 5-11; 2 & 9; 2 & 3-10; 2 & 4-11; 2 & 10; 2 & 3-11; 2 & 11; 3 & 4; 3& 4-5; 3 & 5-6; 3 & 6-7; 3 & 7-8; 3 & 8-9; 3 & 9 & 10; 3 & 10 & 11; 3 & 5; 3 & 4-6; 3 & 5-7; 3 & 6-8; 3 & 7-9; 3 & 8-10; 3 & 9 & 11; 3 & 6; 3 & 4-7; 3 & 5-8; 3 & 6-9; 3 & 7-10; 3 & 8-11; 3 & 7; 3 & 4-8; 3 & 5-9; 3 & 6-10; 3 & 7-11; 3 & 8; 3 & 4-9; 3 & 5-10; 3 & 6-11; 3 & 9; 3 & 4-10; 3 & 5-11; 3 & 10; 3 & 4-11; 3 & 11; 4 & 5; 4 & 5-6; 4 & 6-7; 4 & 7-8; 4 & 8-9; 4 & 9 & 10; 4 & 10-11; 4 & 6; 4 & 5-7; 4 & 6-8; 4 & 7-9; 4 & 8-10; 4 & 9-11; 4 & 7; 4 & 5-8; 4 & 6-9; 4 & 7-10; 4 & 8-11; 4 & 8; 4 & 5-9; 4 & 6-10; 4 & 7-11; 4 & 9; 4 & 5-10; 4 & 6-11; 4 & 10; 4 & 5-11; 4 & 11; 5 & 6; 5 & 6-7; 5 & 7-8; 5 & 8-9; 5 & 9 & 10; 5 & 10-11; 5 & 7; 5 & 6-8; 5 & 7-9; 5 & 8-10; 5 & 9-11; 5 & 8; 5 & 6-9; 5 & 7-10; 5 & 8-11; 5 & 9; 5 & 6-10; 5 & 7-11; 5 & 10; 5 & 6-11; 5 & 11; 6 & 7; 6& 7-8; 6 & 8-9; 6 & 9 & 10; 6 & 10-11; 6 & 8; 6 & 7-9; 6 & 8-10; 6 & 9-11; 6 & 9; 6 & 7-10; 6 & 8-11; 6 & 10; 6 & 7-11; 6 & 11; 7 & 8; 7 & 8-9; 7 & 9 & 10; 7 & 10-11; 7 & 9; 7 & 8-10; 7 & 9-11; 7 & 10; 7 & 8-11; 7 & 11; 8 & 9; 8 & 9-10; 8 & 10-11; 8 & 10; 8 & 9-11; 8 & 11; 9 & 10; 9 & 10-11; or gene numbers 9 & 11 of any of Table 2, 3, 5, 6, 7, 12, 13, 14, 15, 16, 17, 18 or 19.

In some embodiments, the test value incorporating or representing the overall expression of the plurality of test genes is compared to one or more reference values (or index values), and optionally correlated to a poor or good prognosis (e.g., shorter expected post-surgery metastasis-free survival) or an increased or no increased likelihood of response to treatment comprising chemotherapy. In some cases such values are called “scores,” especially in the Examples below. In some embodiments a test value greater than the reference value(s) (or a test value that, relative to the reference value, represents increased expression of the test genes) can be correlated to a poor prognosis and/or increased likelihood of response to treatment comprising chemotherapy. In some embodiments the test value is deemed “greater than” the reference value (e.g., the threshold index value), and thus correlated to a poor prognosis and/or an increased likelihood of response to treatment comprising chemotherapy, if the test value exceeds the reference value by at least some amount (e.g., at least 0.5, 0.75, 0.85, 0.90, 0.95, 1, 2, 3, 4, 5, 6, 7, 8, 9, or 10 or more fold or standard deviations).

For example, the index value may incorporate or represent the gene expression levels found in a normal sample obtained from the patient of interest (including tissue surrounding the cancerous tissue in a biopsy), in which case an expression level in the tumor sample significantly higher than this index value would indicate, e.g., increased likelihood of response to a particular treatment regimen (e.g., a treatment regimen comprising chemotherapy).

Alternatively, the index value may incorporate or represent the average expression level for a set of individuals from a diverse cancer population or a subset of the population. For example, one may determine the average expression level of a gene or gene panel in a random sampling of patients with cancer (e.g., lung cancer). This average expression level may be termed the “threshold index value,” with patients having a test value higher than this value or a test value representing expression higher than the expression represented by the threshold index value (or at least some amount higher than this value) expected to have a better prognosis and/or a greater likelihood of response to a particular treatment regimen (e.g., a treatment regimen comprising chemotherapy) than those having a test value lower than this value.

Alternatively, the index value may incorporate or represent the average expression level of a particular gene or gene panel in a plurality of training patients (e.g., lung cancer patients) with similar outcomes whose clinical and follow-up data are available and sufficient to define and categorize the patients by disease outcome, e.g., response to a particular treatment regimen (e.g., a treatment regimen comprising chemotherapy). See, e.g., Examples, infra. For example, a “poor prognosis index value” or a “good response index value” can be generated from a plurality of training cancer patients characterized as having “poor prognosis” or a “good prognosis/response”, e.g., relatively short expected survival (e.g., overall survival, disease-free survival, distant metastasis-free survival, etc.); complete response, partial response, or stable disease (e.g., by RECIST criteria) after treatment comprising chemotherapy. A “good response index value” or a “poor response index value” can be generated from a plurality of training cancer patients defined as having “good prognosis” or “poor response”, e.g., absence of complete response, partial response, or stable disease (e.g., by RECIST criteria) after treatment comprising chemotherapy. Thus, for example, a good response index value of a particular gene or gene panel may represent the average level of expression of the particular gene or gene panel in patients having a “good response,” whereas a poor response index value of a particular gene or gene panel represents the average level of expression of the particular gene or gene panel in patients having a “poor response.” Thus, if the determined level of expression of a relevant gene or gene panel is closer to the good response index value of the gene or gene panel than to the poor response index value of the gene or gene panel, then it can be concluded that the patient is more likely to have a good response. On the other hand, if the determined level of expression of a relevant gene or gene panel is closer to the poor response index value of the gene or gene panel than to the good response index value of the gene or gene panel, then it can be concluded that the patient is more likely to have a poor response.

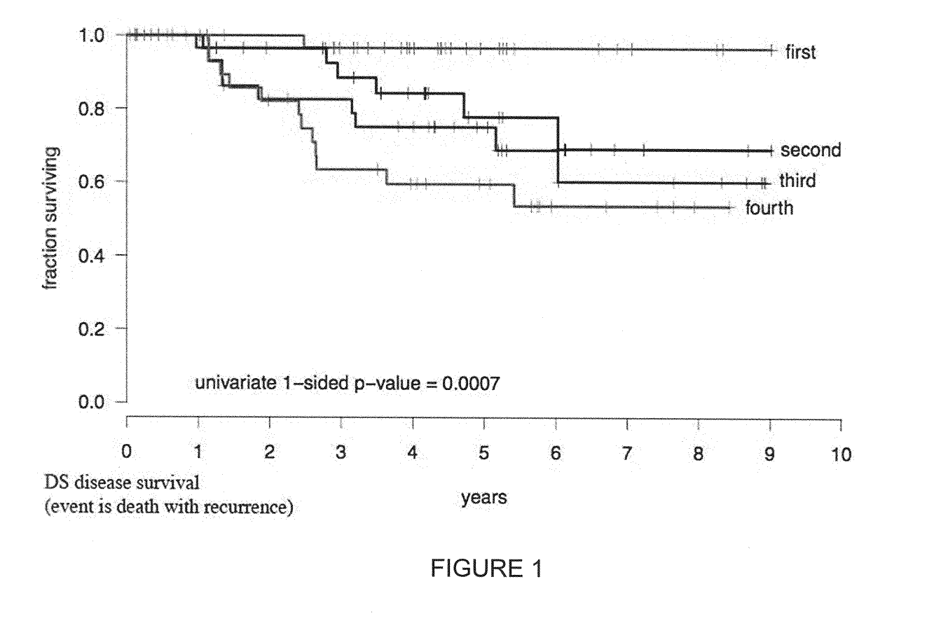

Alternatively index values may be determined thusly: In order to assign patients to risk groups, a threshold value may be set for the cell cycle mean. The optimal threshold value is selected based on the receiver operating characteristic (ROC) curve, which plots sensitivity vs (1−specificity). For each increment of the cell cycle mean, the sensitivity and specificity of the test is calculated using that value as a threshold. The actual threshold will be the value that optimizes these metrics according to the artisan's requirements (e.g., what degree of sensitivity or specificity is desired, etc.). FIG. 1 and the accompanying discussion herein demonstrate determination of a threshold value determined and validated experimentally.

Panels of CCGs (e.g., 2, 3, 4, 5, 6, 7, 8, 9, or 10 or more CCGs) can accurately predict response, as shown in FIG. 1 and Table 20. Those skilled in the art are familiar with various ways of determining the expression of a panel of genes (i.e., a plurality of genes). One may determine the expression of a panel of genes by determining the average expression level (normalized or absolute) of all panel genes in a sample obtained from a particular patient (either throughout the sample or in a subset of cells or a single cell from the sample). Increased expression in this context will mean the average expression is higher than the average expression level of these genes in some reference (e.g., higher than in normal patients; higher than some index value that has been determined to represent the average expression level in a reference population, such as patients with the same cancer; etc.). Alternatively, one may determine the expression of a panel of genes by determining the average expression level (normalized or absolute) of at least a certain number (e.g., 1, 2, 3, 4, 5, 6, 7, 8, 9, 10, 15, 20, 25, 30 or more) or at least a certain proportion (e.g., 10%, 20%, 30%, 40%, 50%, 60%, 70%, 80%, 90%, 95%, 99%, 100%) of the genes in the panel. Alternatively, one may determine the expression of a panel of genes by determining the absolute copy number of the analyte representing each gene in the panel (e.g., mRNA, cDNA, protein) and either total or average these across the genes.

“Response” (e.g., response to a particular treatment regimen) is a well-known term in the art and is used herein according to its known meaning. As an example, the meaning of “response” may be cancer-type dependent, with response in lung cancer meaning something different from response in prostate cancer. However, within each cancer-type and subtype “response” is clearly understood to those skilled in the art. For example, some objective criteria of response include Response Evaluation Criteria In Solid Tumors (RECIST), a set of published rules (e.g., changes in tumor size, etc.) that define when cancer patients improve (“respond”), stay the same (“stabilize”), or worsen (“progression”) during treatments. See, e.g., Eisenhauer et al., EUR. J. CANCER (2009) 45:228-247. “Response” can also include survival metrics (e.g., “disease-free survival” (DFS), “overall survival” (OS), etc). In some cases RECIST criteria can include: (a) Complete response (CR): disappearance of all metastases; (b) Partial response (PR): at least a 30% decrease in the sum of the largest diameter (LD) of the metastatic lesions, taking as reference the baseline sum LD; (c) Stable disease (SD): neither sufficient shrinkage to qualify for PR nor sufficient increase to qualify for PD taking as references the smallest sum LD since the treatment started; (d) Progression (PD): at least a 20% increase in the sum of the LD of the target metastatic lesions taking as reference the smallest sum LD since the treatment started or the appearance of one or more new lesions.

As shown in the Examples below, increased CCG expression correlates well with increased likelihood of response to particular treatments (e.g., treatments comprising chemotherapy). As used herein, “particular treatment” refers to a medical management regimen with at least some defined parameters. These may include administration (including prescription) of particular therapeutic agent alone; a specific combination of agents (e.g., FOLFOX, FOLFIRI); a combination of agents at least comprising a particular agent (e.g., 5-fluorouracil) or subcombination of agents (e.g., platinum compounds with taxanes) together with any other agents or interventions (e.g., surgery, radiation); a surgical or other intervention (e.g., surgical resection of the tumor, radiation therapy); or any combination of these (e.g., surgical resection of the tumor followed by chemotherapy, also known as “adjuvant” chemotherapy). “Chemotherapy” as used herein has its conventional meaning as is well-known in the art. In some embodiments, the particular treatment (e.g., a treatment regimen comprising chemotherapy) comprises a platinum-based compound (e.g., cisplatin, carboplatin, oxaliplatin) paired with a taxane (e.g., docetaxel, paclitaxel) and/or gemcitabine.

For many lung cancer patients and their physicians surgery to remove the tumor (sometimes including surrounding healthy tissue) is the standard of care. Because surgery can cure some patients and adjuvant chemotherapy is debilitating and expensive, the decision whether to undertake adjuvant chemotherapy is more difficult. In some embodiments, increased expression of CCGs correlates with increased likelihood of response to adjuvant chemotherapy (and thus in some embodiments adjuvant chemotherapy is administered, recommended or prescribed if expression of CCGs is increased). In some embodiments, increased expression of a plurality of test genes comprising CCGs, where CCGs are weighted to contribute at least 50% or more to a test value incorporating or representing the expression of the plurality of test genes, correlates with increased likelihood of response to adjuvant chemotherapy (and thus in some embodiments adjuvant chemotherapy is administered, recommended or prescribed if expression of the plurality of test genes is increased).

As used herein, a patient has an “increased likelihood” of some clinical feature or outcome (e.g., response) if the probability of the patient having the feature or outcome exceeds some reference probability or value. The reference probability may be the probability of the feature or outcome across the general relevant patient population. For example, if the probability of response (e.g., to treatment comprising chemotherapy) in the general lung cancer patient population (or some specific subpopulation, e.g., in stage Ia, Ib, or II lung cancer patients) is X % and a particular patient has been determined by the methods of the present invention to have a probability of response of Y %, and if Y>X, then the patient has an “increased likelihood” of response. In some embodiments, the patient has an increased likelihood of response if Y−X=at least 10, 20, 30, 40, 50, 60, 70, 80, or 90. Alternatively, as discussed above, a threshold or reference value may be determined and a particular patient's probability of response may be compared to that threshold or reference. Because predicting response is a prognostic endeavor, “predicting prognosis” will sometimes be used herein to refer to predicting response.

Similarly, prognosis is often used in a relative sense. Often when it is said that a patient has a poor prognosis, this means the patient has a worse prognosis than other (e.g., average) patients (or worse than the patient would have had if the patient had different clinical indications). Thus, unless expressly stated otherwise or the context clearly indicates otherwise, “poor prognosis” includes “poorer prognosis” and “good prognosis” includes “better prognosis.” As discussed elsewhere in this document, prognosis can include a patient's likelihood of cancer recurrence, cancer metastasis, or new primary cancer(s). In these cases, “poor prognosis” means the patient has an “increased likelihood” (as discussed in the preceding paragraph) of one of these clinical outcomes. Prognosis can also include the likelihood of survival (e.g., overall survival, disease-free survival, distant metastasis-free survival, etc.). In these cases, “poor prognosis” means either (a) the patient's (estimated) expected survival time is some certain amount (e.g., 1, 2, 3, 4, 5, 6, 7, 8, 9, 10, 15, or 20 years), which is lower than some reference amount; or (b) the patient has a “decreased likelihood” (as discussed in the preceding paragraph) of survival beyond a certain amount of time (e.g., 1, 2, 3, 4, 5, 6, 7, 8, 9, 10, 15, 20 or more years). The opposite would of course be true for a “good prognosis.”

As shown in Tables 6 & 7, individual CCGs can predict response quite well. Thus some embodiments of the invention comprise determining the expression of a single CCG listed in any of Table 1, 2, 3, 5, 6, 7, 8, 9, 10 or 11 or Panel A, B, C, D, E, F, G, H, J or K and correlating increased expression to increased likelihood of response.

FIG. 1 and Table 20 show that panels of CCGs (e.g., 2, 3, 4, 5, or 6 CCGs) can accurately predict response. Thus in some aspects the invention provides a method of classifying a cancer comprising determining the status of a panel of genes (e.g., a plurality of test genes) comprising a plurality of CCGs. For example, increased expression in a panel of genes (or plurality of test genes) may refer to the average expression level of all panel or test genes in a particular patient being higher than the average expression level of these genes in normal patients (or higher than some index value that has been determined to represent the normal average expression level). Alternatively, increased expression in a panel of genes may refer to increased expression in at least a certain number (e.g., 1, 2, 3, 4, 5, 6, 7, 8, 9, 10, 15, 20, 25, 30 or more) or at least a certain proportion (e.g., 10%, 20%, 30%, 40%, 50%, 60%, 70%, 80%, 90%, 95%, 99%, 100%) of the genes in the panel as compared to the average normal expression level.

In some embodiments the panel comprises at least 3, 4, 5, 6, 7, 8, 9, 10, 15, 20, 25, 30, 35, 40, 45, 50, 70, 80, 90, 100, 200, or more CCGs. In some embodiments the panel comprises at least 10, 15, 20, or more CCGs. In some embodiments the panel comprises between 5 and 100 CCGs, between 7 and 40 CCGs, between 5 and 25 CCGs, between 10 and 20 CCGs, or between 10 and 15 CCGs. In some embodiments CCGs comprise at least a certain proportion of the panel. Thus in some embodiments the panel comprises at least 25%, 30%, 40%, 50%, 60%, 70%, 75%, 80%, 85%, 90%, 95%, 96%, 97%, 98%, or 99% CCGs. In some preferred embodiments the panel comprises at least 10, 15, 20, 25, 30, 35, 40, 45, 50, 70, 80, 90, 100, 200, or more CCGs, and such CCGs constitute of at least 50%, 60%, 70%, preferably at least 75%, 80%, 85%, more preferably at least 90%, 95%, 96%, 97%, 98%, or 99% or more of the total number of genes in the panel. In some embodiments the panel of CCGs comprises the genes in Table 1, 2, 3, 5, 6, 7, 8, 9, 10 or 11; Panel A, B, C, D, E, F, G, H, J or K; or “sub-panels” of Panel F in Tables A′ to E′. In some embodiments the panel comprises at least 2, 3, 4, 5, 6, 7, 8, 9, 10, 11, 12, 13, 14, 15, 20, 25, 30, or more of the genes in Table 1, 2, 3, 5, 6, 7, 8, 9, 10 or 11; Panel A, B, C, D, E, F, G, H, J or K; or “sub-panels” of Panel F in Tables A′ to E′. In some embodiments the invention provides a method of determining prognosis and/or predicting response to a particular treatment regimen (e.g., a regimen comprising chemotherapy), the method comprising determining the status of the CCGs in any one of Table 1, 2, 3, 5, 6, 7, 8, 9, 10 or 11; Panel A, B, C, D, E, F, G, H, J or K; or “sub-panels” of Panel F in Tables A′ to E′ and correlating increased expression of the panel to a poor prognosis and/or increased likelihood of response to the treatment regimen.

Several panels of CCGs (shown in Table 1, 2, 3, 5, 6, 7, 8, 9, 10 or 11; Panel A, B, C, D, E, F, G, H, J or K; or “sub-panels” of Panel F in Tables A′ to E′) are useful in determining prognosis and/or predicting response to particular treatment.

TABLE 8
“Panel C”

| Gene | Entrez |
|---|---|
| Symbol | GeneID |
| AURKA | 6790 |
| BUB1* | 699 |
| CCNB1* | 891 |
| CCNB2* | 9133 |
| CDC2* | 983 |
| CDC20* | 991 |
| CDC45L* | 8318 |
| CDCA8* | 55143 |
| CENPA | 1058 |
| CKS2* | 1164 |
| DLG7* | 9787 |
| DTL* | 51514 |
| FOXM1* | 2305 |
| HMMR* | 3161 |
| KIF23* | 9493 |
| KPNA2 | 3838 |
| MAD2L1* | 4085 |
| MELK | 9833 |
| MYBL2* | 4605 |
| NUSAP1* | 51203 |
| PBK* | 55872 |
| PRC1* | 9055 |
| PTTG1* | 9232 |
| RRM2* | 6241 |
| TIMELESS* | 8914 |
| TPX2* | 22974 |
| TRIP13* | 9319 |
| TTK* | 7272 |
| UBE2C | 11065 |
| UBE2S* | 27338 |
| ZWINT* | 11130 |

*These genes can be used as a 26-gene subset panel (“Panel D”) in some embodiments of the invention.

TABLE 9
“Panel E”

| Name | GeneID |
|---|---|
| ASF1B* | 55723 |
| ASPM* | 259266 |
| BIRC5* | 332 |
| BUB1B* | 701 |
| C18orf24* | 220134 |
| CDC2* | 983 |
| CDC20* | 991 |
| CDCA3* | 83461 |
| CDCA8* | 55143 |
| CDKN3* | 1033 |
| CENPF* | 1063 |
| CENPM* | 79019 |
| CEP55* | 55165 |
| DLGAP5* | 9787 |
| DTL* | 51514 |
| FOXM1* | 2305 |
| KIAA0101* | 9768 |
| KIF11* | 3832 |
| KIF20A* | 10112 |
| KIF4A | 24137 |
| MCM10* | 55388 |
| NUSAP1* | 51203 |
| ORC6L* | 23594 |
| PBK* | 55872 |
| PLK1* | 5347 |
| PRC1* | 9055 |
| PTTG1* | 9232 |
| RAD51* | 5888 |
| RAD54L* | 8438 |
| RRM2* | 6241 |
| TK1* | 7083 |
| TOP2A* | 7153 |

*These genes can be used as a 31-gene subset panel (“Panel F”) in some embodiments of the invention.

TABLE 10
“Panel G”

| | | | |
|---|---|---|---|
| ASF1B*# | Hs00216780_m1 | RRM2*# | Hs00357247_g1 |
| ASPM*# | Hs00411505_m1 | TK1*# | Hs01062125_m1 |
| BUB1B*# | Hs01084828_m1 | TOP2A*# | Hs00172214_m1 |
| C18orf24*# | Hs00536843_m1 | GAPDH\hat{ } | Hs99999905_m1 |
| CDC2*# | Hs00364293_m1 | CLTC** | Hs00191535_m1 |
| CDKN3*# | Hs00193192_m1 | MMADHC** | Hs00739517_g1 |
| CENPF*# | Hs00193201_m1 | PPP2CA** | Hs00427259_m1 |
| CENPM*# | Hs00608780_m1 | PSMA1** | Hs00267631_m1 |
| DTL*# | Hs00978565_m1 | PSMC1** | Hs02386942_g1 |
| CDCA3*# | Hs00229905_m1 | RPL13A** | Hs03043885_g1 |
| KIAA0101*# | Hs00207134_m1 | RPL37** | Hs02340038_g1 |
| KIF11*# | Hs00189698_m1 | RPL38** | Hs00605263_g1 |
| KIF20A*# | Hs00993573_m1 | RPL4** | Hs03044647_g1 |
| KIF4A*# | Hs01020169_m1 | RPL8** | Hs00361285_g1 |
| MCM10*# | Hs00960349_m1 | RPS29** | Hs03004310_g1 |
| NUSAP1*# | Hs01006195_m1 | SLC25A3** | Hs00358082_m1 |
| PBK*# | Hs00218544_m1 | TXNL1** | Hs00355488_m1 |
| PLK1*# | Hs00153444_m1 | UBA52** | Hs03004332_g1 |
| PRC1*# | Hs00187740_m1 | | |
| PTTG1*# | Hs00851754_u1 | | |
| RAD51*# | Hs00153418_m1 | | |
| RAD54L*# | Hs00269177_m1 | | |

*CCP genes (Panel H)
**Housekeeping control genes (Panel I)

TABLE 11
“Panel J”

| | | Entrez | | | Entrez |
|---|---|---|---|---|---|
| Gene Symbol | ABI Assay ID | GeneID | Gene Symbol | ABI Assay ID | GeneID |
| ASF1B*# | Hs00216780_m1 | 55723 | RRM2*# | Hs00357247_g1 | 6241 |
| ASPM*# | Hs00411505_m1 | 259266 | TK1*# | Hs01062125_m1 | 7083 |
| BUB1B*# | Hs01084828_m1 | 701 | TOP2A*# | Hs00172214_m1 | 7153 |
| C18orf24*# | Hs00536843_m1 | 220134 | GAPDH\hat{ } | Hs99999905_m1 | 2597 |
| CDC2*# | Hs00364293_m1 | 983 | CLTC** | Hs00191535_m1 | 1213 |
| CDKN3*# | Hs00193192_m1 | 83461 | MMADHC** | Hs00739517_g1 | 27249 |
| CENPF*# | Hs00193201_m1 | 1033 | PPP2CA** | Hs00427259_m1 | 5515 |
| CENPM*# | Hs00608780_m1 | 1063 | PSMA1** | Hs00267631_m1 | 5682 |
| DTL*# | Hs00978565_m1 | 79019 | PSMC1** | Hs02386942_g1 | 5700 |
| CDCA3*# | Hs00229905_m1 | 51514 | RPL13A** | Hs03043885_g1 | 23521 |
| KIAA0101*# | Hs00207134_m1 | 9768 | RPL37** | Hs02340038_g1 | 6167 |
| KIF11*# | Hs00189698_m1 | 3832 | RPL38** | Hs00605263_g1 | 6169 |
| KIF20A*# | Hs00993573_m1 | 10112 | RPL4** | Hs03044647_g1 | 6124 |
| MCM10*# | Hs00960349_m1 | 55388 | RPL8** | Hs00361285_g1 | 6132 |
| NUSAP1*# | Hs01006195_m1 | 51203 | RPS29** | Hs03004310_g1 | 6235 |
| PBK*# | Hs00218544_m1 | 55872 | SLC25A3** | Hs00358082_m1 | 6515 |
| PLK1*# | Hs00153444_m1 | 5347 | TXNL1** | Hs00355488_m1 | 9352 |
| PRC1*# | Hs00187740_m1 | 9055 | UBA52** | Hs03004332_g1 | 7311 |
| PTTG1*# | Hs00851754_u1 | 9232 | | | |
| RAD51*# | Hs00153418_m1 | 5888 | | | |
| RAD54L*# | Hs00269177_m1 | 8438 | | | |

*CCP genes (Panel K)
**Housekeeping control genes
\hat{ }Internal control gene

Similar to Tables 2 to 7 above, the CCP genes in Tables 10 & 11 were ranked according to correlation to the CCP mean and according to independent predictive value (p-value). Rankings according to correlation to the mean are shown in Tables 12 to 14 below. Rankings according to p-value are shown in Tables 15 & 16 below.

TABLE 12

| Gene # | Gene Symbol |
|---|---|
| 1 | KIF4A |
| 2 | CDC2 |
| 3 | PRC1 |
| 4 | TOP2A |
| 5 | KIF20A |
| 6 | BUB1B |
| 7 | CDKN3 |
| 8 | PTTG1 |
| 9 | NUSAP1 |
| 10 | KIF11 |
| 11 | ASPM |
| 12 | RRM2 |
| 13 | CENPF |
| 14 | KIAA0101 |
| 15 | PBK |
| 16 | MCM10 |
| 17 | RAD51 |
| 18 | CDCA3 |
| 19 | ASF1B |
| 20 | DTL |
| 21 | PLK1 |
| 22 | CENPM |
| 23 | TK1 |
| 24 | C18orf24 |
| 25 | RAD54L |

TABLE 13

| Gene # | Gene Symbol |
|---|---|
| 1 | CDKN3 |
| 2 | CDC2 |
| 3 | KIF11 |
| 4 | KIAA0101 |
| 5 | NUSAP1 |
| 6 | CENPF |
| 7 | ASPM |
| 8 | BUB1B |
| 9 | RRM2 |
| 10 | KIF20A |
| 11 | PLK1 |
| 12 | TOP2A |
| 13 | TK1 |
| 14 | PBK |
| 15 | ASF1B |
| 16 | C18orf24 |
| 17 | RAD54L |
| 18 | PTTG1 |
| 19 | KIF4A |
| 20 | CDCA3 |
| 21 | MCM10 |
| 22 | PRC1 |
| 23 | DTL |
| 24 | RAD51 |
| 25 | CENPM |

TABLE 14

| Gene # | Gene Symbol |
|---|---|
| 1 | ASPM |
| 2 | KIF11 |
| 3 | MCM10 |
| 4 | PRC1 |
| 5 | BUB1B |
| 6 | NUSAP1 |
| 7 | C18orf24 |
| 8 | PLK1 |
| 9 | CDKN3 |
| 10 | RRM2 |
| 11 | RAD51 |
| 12 | RAD54L |
| 13 | CDC2 |
| 14 | CENPF |
| 15 | TOP2A |
| 16 | KIF20A |
| 17 | KIAA0101 |
| 18 | CDCA3 |
| 19 | ASF1B |
| 20 | CENPM |
| 21 | TK1 |
| 22 | PBK |
| 23 | PTTG1 |
| 24 | DTL |
| 25 | KIF4A |

TABLE 15

| Gene # | Gene Symbol |
|---|---|
| 1 | NUSAP1 |
| 2 | CDC2 |
| 3 | RRM2 |
| 4 | PTTG1 |
| 5 | PBK |
| 6 | PRC1 |
| 7 | DTL |
| 8 | ASF1B |
| 9 | ASPM |
| 10 | BUB1B |
| 11 | C18orf24 |
| 12 | CDCA3 |
| 13 | CDKN3 |
| 14 | CENPF |
| 15 | CENPM |
| 16 | KIAA0101 |
| 17 | KIF11 |
| 18 | KIF20A |
| 19 | KIF4A |
| 20 | MCM10 |
| 21 | PLK1 |
| 22 | RAD51 |
| 23 | RAD54L |
| 24 | TK1 |
| 25 | TOP2A |

TABLE 16

| Gene # | Gene Symbol |
|---|---|
| 1 | MCM10 |
| 2 | ASPM |
| 3 | CENPF |
| 4 | TOP2A |
| 5 | NUSAP1 |
| 6 | CDKN3 |
| 7 | KIF11 |
| 8 | KIF20A |
| 9 | BUB1B |
| 10 | RAD54L |
| 11 | TK1 |
| 12 | DTL |
| 13 | PRC1 |
| 14 | PTTG1 |
| 15 | CDC2 |
| 16 | PLK1 |
| 17 | C18orf24 |
| 18 | RRM2 |
| 19 | CENPM |
| 20 | RAD51 |
| 21 | KIAA0101 |
| 22 | CDCA3 |
| 23 | PBK |
| 24 | ASF1B |
| 25 | KIF4A |

The rankings of each gene according to correlation to the mean (Tables 2, 3 & 5) and p-value (Tables 6 & 7) were used to derive two different combination rankings Table 17 ranks the CCP genes of Table 10 according to the highest unweighted combination score calculated by the following formula: Combination score for each gene=(1/(correlation in Table 2))+(1/(correlation in Table 3))+(1/(correlation in Table 5))+(1/(p-value in Table 6))+(1/(p-value in Table 7)). Table 18 ranks the CCP genes of Table 10 according to the highest weighted combination score (which gives greater weight to p-value over correlation to the mean) calculated by the following formula: Combination score for each gene=(2/(correlation in Table 2))+(3/(correlation in Table 3))+(5/(correlation in Table 5))+(7/(p-value in Table 6))+(10/(p-value in Table 7)).

TABLE 17

| Gene # | Gene Symbol |
|---|---|
| 1 | NUSAP1 |
| 2 | MCM10 |
| 3 | ASPM |
| 4 | CDC2 |
| 5 | KIF11 |
| 6 | CDKN3 |
| 7 | CENPF |
| 8 | KIF4A |
| 9 | PRC1 |
| 10 | BUB1B |
| 11 | RRM2 |
| 12 | TOP2A |
| 13 | PTTG1 |
| 14 | KIF20A |
| 15 | KIAA0101 |
| 16 | PLK1 |
| 17 | PBK |
| 18 | C18orf24 |
| 19 | RAD54L |
| 20 | DTL |
| 21 | TK1 |
| 22 | RAD51 |
| 23 | ASF1B |
| 24 | CDCA3 |
| 25 | CENPM |

TABLE 18

| Gene # | Gene Symbol |
|---|---|
| 1 | NUSAP1 |
| 2 | CDC2 |
| 3 | KIF11 |
| 4 | ASPM |
| 5 | CDKN3 |
| 6 | BUB1B |
| 7 | PRC1 |
| 8 | RRM2 |
| 9 | CENPF |
| 10 | TOP2A |
| 11 | KIF20A |
| 12 | PTTG1 |
| 13 | MCM10 |
| 14 | KIAA0101 |
| 15 | PBK |
| 16 | PLK1 |
| 17 | DTL |
| 18 | KIF4A |
| 19 | RAD51 |
| 20 | C18orf24 |
| 21 | ASF1B |
| 22 | CDCA3 |
| 23 | TK1 |
| 24 | RAD54L |
| 25 | CENPM |

Analogous to Tables 2 to 7 and Tables 15 & 16 above, the CCP genes in Panel F of Table 9 were ranked according to independent predictive value (p-value) in the study reported as Example 3 below. These rankings are shown in Table 19 below.

TABLE 19

| | Gene | Univariate |
|---|---|---|
| Gene # | Symbol | p-value |
| 1 | C18orf24 | 1.73E−05 |
| 2 | KIF11 | 5.63E−05 |
| 3 | PTTG1 | 6.13E−05 |
| 4 | PBK | 9.10E−05 |
| 5 | CENPF | 1.38E−04 |
| 6 | RAD54L | 1.46E−04 |
| 7 | CEP55 | 3.21E−04 |
| 8 | ORC6L | 4.58E−04 |
| 9 | RRM2 | 4.69E−04 |
| 10 | CDKN3 | 4.89E−04 |
| 11 | DLGAP5 | 5.60E−04 |
| 12 | RAD51 | 7.08E−04 |
| 13 | DTL | 7.88E−04 |
| 14 | KIF20A | 7.98E−04 |
| 15 | FOXM1 | 1.25E−03 |
| 16 | ASPM | 2.37E−03 |
| 17 | BUB1B | 2.54E−03 |
| 18 | CDCA8 | 2.62E−03 |
| 19 | CDC20 | 4.23E−03 |
| 20 | KIAA0101 | 5.08E−03 |
| 21 | BIRC5 | 6.89E−03 |
| 22 | PRC1 | 7.10E−03 |
| 23 | PLK1 | 7.11E−03 |
| 24 | MCM10 | 9.37E−03 |
| 25 | TOP2A | 1.00E−02 |
| 26 | CDC2 | 1.08E−02 |
| 27 | TK1 | 1.15E−02 |
| 28 | CDCA3 | 1.41E−02 |
| 29 | NUSAP1 | 2.48E−02 |
| 30 | CENPM | 3.42E−02 |
| 31 | ASF1B | 4.33E−02 |

In CCG signatures the particular CCGs assayed is often not as important as the total number of CCGs. The number of CCGs assayed can vary depending on many factors, e.g., technical constraints, cost considerations, the classification being made, the cancer being tested, the desired level of predictive power, etc. Increasing the number of CCGs assayed in a panel according to the invention is, as a general matter, advantageous because, e.g., a larger pool of mRNAs to be assayed means less “noise” caused by outliers and less chance of an assay error throwing off the overall predictive power of the test. However, cost and other considerations will generally limit this number and finding the optimal number of CCGs for a signature is desirable.

It has been discovered that the predictive power of a CCG signature often ceases to increase significantly beyond a certain number of CCGs. In order to determine the optimal number of cell cycle genes for the signature, the predictive power of the mean was tested for randomly selected sets of from 1 to 30 of the CCGs in Panel C (FIG. 1). This demonstrates, for some embodiments of the invention, a threshold number of CCGs in a panel (10, 15, or between 10 and 15) that provides significantly improved predictive power. In some embodiments even smaller panels of CCGs are sufficient to prognose disease outcome and/or predict therapy response/benefit (e.g., “sub-panels” of Panel F in Tables A′ to E′). To evaluate how even smaller subsets of a larger CCG set (i.e., smaller CCG subpanels) performed, the inventors compared how well the CCGs from Panel C predicted outcome as a function of the number of CCGs included in the signature (FIG. 1). As shown in Table 20 below and FIG. 1, small CCG signatures (e.g., 2, 3, 4, 5, 6 CCGS, etc.) are significant predictors.

TABLE 20

| # of CCGs | Mean of log10 (p-value)* |
|---|---|
| 1 | −3.579 |
| 2 | −4.279 |
| 3 | −5.049 |
| 4 | −5.473 |
| 5 | −5.877 |
| 6 | −6.228 |

*For 1000 randomly drawn subsets, size 1 through 6, of CCGs.

Tables A′ to E′, submitted as part of this description in electronic form, further illustrate this feature of the invention by showing the predictive power (both univariate and multivariate p-value) of numerous sub-panels chosen from Panel F. As can be seen, each 2-gene and 3-gene sub-panel chosen from Panel F is significantly predictive of lung cancer prognosis in the cohorts described in Examples 1-3. The same is true for all 4-gene, 5-gene and 6-gene sub-panels chosen from the top 10 genes in Panel F (i.e., from the genes in Panel F ranked according to p-value as in Table 19). Thus, in each embodiment of the invention described in this document, there is a further embodiment in which the panel of genes (or the plurality of test genes, etc.) comprises a sub-panel of any of Tables A′ to E′. By way of non-limiting example, the invention provides a method of determining the prognosis of a patient having lung cancer or the likelihood of cancer recurrence in said patient, comprising: (1) obtaining a sample from said patient; (2) determining the expression levels of a panel of genes in said sample, wherein said panel comprises a sub-panel of Panel F chosen from any of Tables A′ to E′; (3) providing a test value by (i) weighting the determined expression of each of a plurality of test genes selected from said panel of genes with a predefined coefficient, and (ii) combining the weighted expression to provide said test value, wherein the genes of said sub-panel are weighted (e.g., collectively) to contribute at least 25% of the test value; and (4) classifying said patient as having a poor or a good prognosis or an increased or not increased likelihood of cancer recurrence based at least in part on said test value.

In some embodiments, the optimal number of CCGs in a signature (n_{O}) can be found wherever the following is true

(Pₙ₊₁−Pₙ)<C_{O},

wherein P is the predictive power (i.e., Pₙ is the predictive power of a signature with n genes and Pₙ₊₁ is the predictive power of a signature with n genes plus one) and C_{O} is some optimization constant. Predictive power can be defined in many ways known to those skilled in the art including, but not limited to, the signature's p-value. C_{O} can be chosen by the artisan based on his or her specific constraints. For example, if cost is not a critical factor and extremely high levels of sensitivity and specificity are desired, C_{O} can be set very low such that only trivial increases in predictive power are disregarded. On the other hand, if cost is decisive and moderate levels of sensitivity and specificity are acceptable, C_{O} can be set higher such that only significant increases in predictive power warrant increasing the number of genes in the signature.

Alternatively, a graph of predictive power as a function of gene number may be plotted (as in FIG. 1) and the second derivative of this plot taken. The point at which the second derivative decreases to some predetermined value (C_{O}′) may be the optimal number of genes in the signature.

FIG. 1 illustrates the empirical determination of optimal numbers of CCGs in CCG panels of the invention. Randomly selected subsets of the 31 CCGs in Panel F were tested as distinct CCG signatures and predictive power (i.e., p-value) was determined for each. As FIG. 1 shows, p-values ceased to improve significantly between about 10 and about 15 CCGs, thus indicating that, in some embodiments, an optimal number of CCGs in a prognostic panel is from about 10 to about 15. Thus some embodiments of the invention provide a method of predicting prognosis (or likelihood of response to a particular treatment regimen) in a patient having lung cancer comprising determining the status of a panel of genes, wherein the panel comprises between about 10 and about 15 CCGs and increased expression of the CCGs indicates a poor prognosis (or an increased likelihood of response to the particular treatment, e.g., treatment comprising chemotherapy). In some embodiments the panel comprises between about 10 and about 15 CCGs and the CCGs constitute at least 90% of the panel (or are weighted to contribute at least 75%). In other embodiments the panel comprises CCGs plus one or more additional markers that significantly increase the predictive power of the panel (i.e., make the predictive power significantly better than if the panel consisted of only the CCGs). Any other combination of CCGs (including any of those listed in Table 1, 2, 3, 5, 6, 7, 8, 9, 10 or 11; Panel A, B, C, D, E, F, G, H, J or K; or “sub-panels” of Panel F in Tables A′ to E′) can be used to practice the invention.

In some embodiments the panel comprises at least 3, 4, 5, 6, 7, 8, 9, 10, 15, 20, 25, 30, 35, 40, 45, 50 or more CCGs. In some embodiments the panel comprises between 5 and 100 CCGs, between 7 and 40 CCGs, between 5 and 25 CCGs, between 10 and 20 CCGs, or between 10 and 15 CCGs. In some embodiments CCGs comprise at least a certain proportion of the panel. Thus in some embodiments the panel comprises at least 25%, 30%, 40%, 50%, 60%, 70%, 75%, 80%, 85%, 90%, 95%, 96%, 97%, 98%, or 99% CCGs. In some embodiments the CCGs are any of the genes listed in Table 1, 2, 3, 5, 6, 7, 8, 9, 10 or 11; Panel A, B, C, D, E, F, G, H, J or K; or “sub-panels” of Panel F in Tables A′ to E′. In some embodiments the panel comprises at least 2, 3, 4, 5, 6, 7, 8, 9, 10, 15, 20, 25, 30, 35, 40, 45, 50 or more genes in any of Table 1, 2, 3, 5, 6, 7, 8, 9, 10 or 11; Panel A, B, C, D, E, F, G, H, J or K; or “sub-panels” of Panel F in Tables A′ to E′. In some embodiments the panel comprises all of the genes in any of Table 1, 2, 3, 5, 6, 7, 8, 9, 10 or 11; Panel A, B, C, D, E, F, G, H, J or K; or “sub-panels” of Panel F in Tables A′ to E′.

As mentioned above, many of the CCGs of the invention have been analyzed to determine their correlation to the CCG mean and also to determine their relative predictive value within a panel (see Tables 2, 3, 5, 6, 7, 12, 13, 14, 15, 16, 17, 18 & 19). Thus in some embodiments the plurality of test genes comprises at least some number of CCGs (e.g., at least 3, 4, 5, 6, 7, 8, 9, 10, 15, 20, 25, 30, 35, 40, 45, 50 or more CCGs) and this plurality of CCGs comprises the top 2, 3, 4, 5, 6, 7, 8, 9, 10, 11, 12, 13, 14, 15, 20, 25, 30, 35, 40 or more CCGs listed in Table 2, 3, 5, 6, 7, 12, 13, 14, 15, 16, 17, 18 or 19. In some embodiments the plurality of test genes comprises at least some number of CCGs (e.g., at least 3, 4, 5, 6, 7, 8, 9, 10, 15, 20, 25, 30, 35, 40, 45, 50 or more CCGs) and this plurality of CCGs comprises at least 1, 2, 3, 4, 5, 6, 7, 8, 9, 10, 15, or 20 of the following genes: ASPM, BIRC5, BUB1B, CCNB2, CDC2, CDC20, CDCA8, CDKN3, CENPF, DLGAP5, FOXM1, KIAA0101, KIF11, KIF2C, KIF4A, MCM10, NUSAP1, PRC1, RACGAP1, and TPX2. In some embodiments the plurality of test genes comprises at least some number of CCGs (e.g., at least 3, 4, 5, 6, 7, 8, 9, 10, 15, 20, 25, 30, 35, 40, 45, 50 or more CCGs) and this plurality of CCGs comprises any one, two, three, four, five, six, seven, eight, nine, or ten or all of gene numbers 1 & 2, 1 to 3, 1 to 4, 1 to 5, 1 to 6, 1 to 7, 1 to 8, 1 to 9, or 1 to 10 of any of Table 2, 3, 5, 6, 7, 12, 13, 14, 15, 16, 17, 18 or 19. In some embodiments the plurality of test genes comprises at least some number of CCGs (e.g., at least 3, 4, 5, 6, 7, 8, 9, 10, 15, 20, 25, 30, 35, 40, 45, 50 or more CCGs) and this plurality of CCGs comprises any one, two, three, four, five, six, seven, eight, or nine or all of gene numbers 2 & 3, 2 to 4, 2 to 5, 2 to 6, 2 to 7, 2 to 8, 2 to 9, or 2 to 10 of any of Table 2, 3, 5, 6, 7, 12, 13, 14, 15, 16, 17, 18 or 19. In some embodiments the plurality of test genes comprises at least some number of CCGs (e.g., at least 3, 4, 5, 6, 7, 8, 9, 10, 15, 20, 25, 30, 35, 40, 45, 50 or more CCGs) and this plurality of CCGs comprises any one, two, three, four, five, six, seven, or eight or all of gene numbers 3 & 4, 3 to 5, 3 to 6, 3 to 7, 3 to 8, 3 to 9, or 3 to 10 of any of Table 2, 3, 5, 6, 7, 12, 13, 14, 15, 16, 17, 18 or 19. In some embodiments the plurality of test genes comprises at least some number of CCGs (e.g., at least 3, 4, 5, 6, 7, 8, 9, 10, 15, 20, 25, 30, 35, 40, 45, 50 or more CCGs) and this plurality of CCGs comprises any one, two, three, four, five, six, or seven or all of gene numbers 4 & 5, 4 to 6, 4 to 7, 4 to 8, 4 to 9, or 4 to 10 of any of Table 2, 3, 5, 6, 7, 12, 13, 14, 15, 16, 17, 18 or 19. In some embodiments the plurality of test genes comprises at least some number of CCGs (e.g., at least 3, 4, 5, 6, 7, 8, 9, 10, 15, 20, 25, 30, 35, 40, 45, 50 or more CCGs) and this plurality of CCGs comprises any one, two, three, four, five, six, seven, eight, nine, 10, 11, 12, 13, 14, or 15 or all of gene numbers 1 & 2, 1 to 3, 1 to 4, 1 to 5, 1 to 6, 1 to 7, 1 to 8, 1 to 9, 1 to 10, 1 to 11, 1 to 12, 1 to 13, 1 to 14, or 1 to 15 of any of Table 2, 3, 5, 6, 7, 12, 13, 14, 15, 16, 17, 18 or 19.

In some embodiments the invention provides an method of determining a lung cancer patient's prognosis or the likelihood of the patient responding to a particular treatment comprising: (1) obtaining the measured expression levels of a plurality of genes comprising a plurality of CCGs (e.g., genes in Table 1, 2, 3, 5, 6, 7, 8, 9, 10 or 11; Panel A, B, C, D, E, F, G, H, J or K; or “sub-panels” of Panel F in Tables A′ to E′) in a sample from the patient; (2) obtaining a clinical score for the patient comprising (or reflecting) one or more clinical parameters relevant to the patient's lung cancer (e.g., age, gender, smoking, stage, treatment, tumor size, pleural invasion); (3) deriving a combined test value from the measured levels obtained in (1) and the clinical score obtained in (2); (4) comparing the combined test value to a combined reference value derived from measured expression levels of the plurality of genes and a clinical score comprising (or reflecting) the one or more clinical parameters in a reference population of patients; and (5)(a) correlating a combined test value greater than the combined reference value to a poor prognosis (or increased likelihood of response to a particular treatment) or (5)(b) correlating a combined test value equal to or less than the combined reference value to a good prognosis (or decreased likelihood of response to a particular treatment).

In some embodiments the combined score includes CCP score and any single parameter or combination of age, gender, smoking, stage, treatment, tumor size, and pleural invasion (which single or combination of clinical parameters can be termed the “clinical score” component of the combined score). CCP, age and tumor size can be a continuous numeric variable. Gender, smoking, treatment, and pleural invasion can be a binary numeric variable (e.g., yes=X, no=Y). Tumor stage can be a numeric variable with a particular value assigned to any particular clinical stage (example shown below).

In some embodiments the combined score is calculated according to the following formula:

Combined Score=A*(CCP Score)+B*(Clinical Score) (1)

In some embodiments the clinical score is the patient's score according to a clinical nomogram for lung cancer prognosis (or for predicting response to a particular treatment). In some embodiments the combined score is calculated according to the following modified version of Formula 1:

Combined Score=C*(A*(CCP score)+B*(clinical score))+D (2)

wherein C and D can each be additional variables (e.g., expression of other genes) with their own coefficients, additional functions, or predetermined constants. In some such embodiments C=20 and D=15.

In some embodiments CCP score is the unweighted mean of C_{T} values for expression of the CCP genes being analyzed (e.g., any gene(s) in Table 1, 2, 3, 5, 6, 7, 8, 9, 10 or 11; Panel A, B, C, D, E, F, G, H, J or K; or “sub-panels” of Panel F in Tables A′ to E′), optionally normalized by the unweighted mean of the control genes so that higher values indicate higher expression (in some embodiments one unit is equivalent to a two-fold change in expression). In some embodiments the CCP score ranges from −8 to 8 or from −1.6 to 3.7.

In one particular embodiment, clinical score is represented by the numeric value assigned the patient's tumor stage as shown below:

| IASLC 7th Edition | Numeric |
|---|---|
| Pathologic Stage | Stage |
| IA = | 1 |
| IB = | 2 |
| IIA = | 3 |
| IIB = | 4 |

In one embodiment of the invention utilizing Formula 1 (or Formula 2 wherein C and D are each 0), A=0.34 and B=0.49. In another embodiment utilizing Formula 1 (or Formula 2 wherein C and D are each 0), A=0.33 and B=0.52. In one embodiment utilizing Formula 1 (or Formula 2 wherein C and D are each 0), A=0.33 and B=0.52 and the “clinical score” comprises (or consists of) pathologic stage as shown above. In one embodiment utilizing Formula 2, A=0.33, B=0.52, C=20, D=15 and the “clinical score” of B comprises (or consists of or consists essentially of) pathologic stage as shown above.

In some embodiments A=0.34 & B=0.49; A=0.95, B=0.61; A=0.57 & B=0.39; or A=0.58 & B=0.41. In some embodiments, A, B, C and/or D is within rounding of these values (e.g., A is between 0.945 and 0.954 or between 0.325 and 0.334, B is between 0.515 and 0.524, etc.). In some embodiments, A, B, C and/or D is within ±1%, ±2%, ±3%, ±4%, ±5%, ±10%, ±15%, ±20%, ±25%, ±30%, ±35%, ±40%, ±45%, ±50%, of these values (e.g., A is between 0.29 and 0.37, B is between 0.46 and 0.58, etc.). In some cases a formula may not have all of the specified coefficients (and thus not incorporate the corresponding variable(s)). In some embodiments A is between 0.9 and 1, 0.9 and 0.99, 0.9 and 0.95, 0.85 and 0.95, 0.86 and 0.94, 0.87 and 0.93, 0.88 and 0.92, 0.89 and 0.91, 0.85 and 0.9, 0.8 and 0.95, 0.8 and 0.9, 0.8 and 0.85, 0.75 and 0.99, 0.75 and 0.95, 0.75 and 0.9, 0.75 and 0.85, or between 0.75 and 0.8. In some embodiments B is between 0.40 and 1, 0.45 and 0.99, 0.45 and 0.95, 0.55 and 0.8, 0.55 and 0.7, 0.55 and 0.65, 0.59 and 0.63, or between 0.6 and 0.62.

In some embodiments A is between 0.1 and 0.2, 0.3, 0.4, 0.5, 0.6, 0.7, 0.8, 0.9, 1, 1.5, 2, 2.5, 3, 3.5, 4, 4.5, 5, 6, 7, 8, 9, 10, 11, 12, 13, 14, 15, or 20; or between 0.2 and 0.3, 0.4, 0.5, 0.6, 0.7, 0.8, 0.9, 1, 1.5, 2, 2.5, 3, 3.5, 4, 4.5, 5, 6, 7, 8, 9, 10, 11, 12, 13, 14, 15, or 20; or between 0.3 and 0.4, 0.5, 0.6, 0.7, 0.8, 0.9, 1, 1.5, 2, 2.5, 3, 3.5, 4, 4.5, 5, 6, 7, 8, 9, 10, 11, 12, 13, 14, 15, or 20; or between 0.4 and 0.5, 0.6, 0.7, 0.8, 0.9, 1, 1.5, 2, 2.5, 3, 3.5, 4, 4.5, 5, 6, 7, 8, 9, 10, 11, 12, 13, 14, 15, or 20; or between 0.5 and 0.6, 0.7, 0.8, 0.9, 1, 1.5, 2, 2.5, 3, 3.5, 4, 4.5, 5, 6, 7, 8, 9, 10, 11, 12, 13, 14, 15, or 20; or between 0.6 and 0.7, 0.8, 0.9, 1, 1.5, 2, 2.5, 3, 3.5, 4, 4.5, 5, 6, 7, 8, 9, 10, 11, 12, 13, 14, 15, or 20; or between 0.7 and 0.8, 0.9, 1, 1.5, 2, 2.5, 3, 3.5, 4, 4.5, 5, 6, 7, 8, 9, 10, 11, 12, 13, 14, 15, or 20; or between 0.8 and 0.9, 1, 1.5, 2, 2.5, 3, 3.5, 4, 4.5, 5, 6, 7, 8, 9, 10, 11, 12, 13, 14, 15, or 20; or between 0.9 and 1, 1.5, 2, 2.5, 3, 3.5, 4, 4.5, 5, 6, 7, 8, 9, 10, 11, 12, 13, 14, 15, or 20; or between 1 and 1.5, 2, 2.5, 3, 3.5, 4, 4.5, 5, 6, 7, 8, 9, 10, 11, 12, 13, 14, 15, or 20; or between 1.5 and 2, 2.5, 3, 3.5, 4, 4.5, 5, 6, 7, 8, 9, 10, 11, 12, 13, 14, 15, or 20; or between 2 and 2.5, 3, 3.5, 4, 4.5, 5, 6, 7, 8, 9, 10, 11, 12, 13, 14, 15, or 20; or between 2.5 and 3, 3.5, 4, 4.5, 5, 6, 7, 8, 9, 10, 11, 12, 13, 14, 15, or 20; or between 3 and 3.5, 4, 4.5, 5, 6, 7, 8, 9, 10, 11, 12, 13, 14, 15, or 20; or between 3.5 and 4, 4.5, 5, 6, 7, 8, 9, 10, 11, 12, 13, 14, 15, or 20; or between 4 and 4.5, 5, 6, 7, 8, 9, 10, 11, 12, 13, 14, 15, or 20; or between 4.5 and 5, 6, 7, 8, 9, 10, 11, 12, 13, 14, 15, or 20; or between 5 and 6, 7, 8, 9, 10, 11, 12, 13, 14, 15, or 20; or between 6 and 7, 8, 9, 10, 11, 12, 13, 14, 15, or 20; or between 7 and 8, 9, 10, 11, 12, 13, 14, 15, or 20; or between 8 and 9, 10, 11, 12, 13, 14, 15, or 20; or between 9 and 10, 11, 12, 13, 14, 15, or 20; or between 10 and 11, 12, 13, 14, 15, or 20; or between 11 and 12, 13, 14, 15, or 20; or between 12 and 13, 14, 15, or 20; or between 13 and 14, 15, or 20; or between 14 and 15, or 20; or between 15 and 20; B is between 0.1 and 0.2, 0.3, 0.4, 0.5, 0.6, 0.7, 0.8, 0.9, 1, 1.5, 2, 2.5, 3, 3.5, 4, 4.5, 5, 6, 7, 8, 9, 10, 11, 12, 13, 14, 15, or 20; or between 0.2 and 0.3, 0.4, 0.5, 0.6, 0.7, 0.8, 0.9, 1, 1.5, 2, 2.5, 3, 3.5, 4, 4.5, 5, 6, 7, 8, 9, 10, 11, 12, 13, 14, 15, or 20; or between 0.3 and 0.4, 0.5, 0.6, 0.7, 0.8, 0.9, 1, 1.5, 2, 2.5, 3, 3.5, 4, 4.5, 5, 6, 7, 8, 9, 10, 11, 12, 13, 14, 15, or 20; or between 0.4 and 0.5, 0.6, 0.7, 0.8, 0.9, 1, 1.5, 2, 2.5, 3, 3.5, 4, 4.5, 5, 6, 7, 8, 9, 10, 11, 12, 13, 14, 15, or 20; or between 0.5 and 0.6, 0.7, 0.8, 0.9, 1, 1.5, 2, 2.5, 3, 3.5, 4, 4.5, 5, 6, 7, 8, 9, 10, 11, 12, 13, 14, 15, or 20; or between 0.6 and 0.7, 0.8, 0.9, 1, 1.5, 2, 2.5, 3, 3.5, 4, 4.5, 5, 6, 7, 8, 9, 10, 11, 12, 13, 14, 15, or 20; or between 0.7 and 0.8, 0.9, 1, 1.5, 2, 2.5, 3, 3.5, 4, 4.5, 5, 6, 7, 8, 9, 10, 11, 12, 13, 14, 15, or 20; or between 0.8 and 0.9, 1, 1.5, 2, 2.5, 3, 3.5, 4, 4.5, 5, 6, 7, 8, 9, 10, 11, 12, 13, 14, 15, or 20; or between 0.9 and 1, 1.5, 2, 2.5, 3, 3.5, 4, 4.5, 5, 6, 7, 8, 9, 10, 11, 12, 13, 14, 15, or 20; or between 1 and 1.5, 2, 2.5, 3, 3.5, 4, 4.5, 5, 6, 7, 8, 9, 10, 11, 12, 13, 14, 15, or 20; or between 1.5 and 2, 2.5, 3, 3.5, 4, 4.5, 5, 6, 7, 8, 9, 10, 11, 12, 13, 14, 15, or 20; or between 2 and 2.5, 3, 3.5, 4, 4.5, 5, 6, 7, 8, 9, 10, 11, 12, 13, 14, 15, or 20; or between 2.5 and 3, 3.5, 4, 4.5, 5, 6, 7, 8, 9, 10, 11, 12, 13, 14, 15, or 20; or between 3 and 3.5, 4, 4.5, 5, 6, 7, 8, 9, 10, 11, 12, 13, 14, 15, or 20; or between 3.5 and 4, 4.5, 5, 6, 7, 8, 9, 10, 11, 12, 13, 14, 15, or 20; or between 4 and 4.5, 5, 6, 7, 8, 9, 10, 11, 12, 13, 14, 15, or 20; or between 4.5 and 5, 6, 7, 8, 9, 10, 11, 12, 13, 14, 15, or 20; or between 5 and 6, 7, 8, 9, 10, 11, 12, 13, 14, 15, or 20; or between 6 and 7, 8, 9, 10, 11, 12, 13, 14, 15, or 20; or between 7 and 8, 9, 10, 11, 12, 13, 14, 15, or 20; or between 8 and 9, 10, 11, 12, 13, 14, 15, or 20; or between 9 and 10, 11, 12, 13, 14, 15, or 20; or between 10 and 11, 12, 13, 14, 15, or 20; or between 11 and 12, 13, 14, 15, or 20; or between 12 and 13, 14, 15, or 20; or between 13 and 14, 15, or 20; or between 14 and 15, or 20; or between 15 and 20. In some embodiments, A, B, and/or C is within rounding of any of these values (e.g., A is between 0.45 and 0.54, etc.).

The results of any analyses according to the invention will often be communicated to physicians, genetic counselors and/or patients (or other interested parties such as researchers) in a transmittable form that can be communicated or transmitted to any of the above parties. Such a form can vary and can be tangible or intangible. The results can be embodied in descriptive statements, diagrams, photographs, charts, images or any other visual forms. For example, graphs showing expression or activity level or sequence variation information for various genes can be used in explaining the results. Diagrams showing such information for additional target gene(s) are also useful in indicating some testing results. The statements and visual forms can be recorded on a tangible medium such as papers, computer readable media such as floppy disks, compact disks, etc., or on an intangible medium, e.g., an electronic medium in the form of email or website on internet or intranet. In addition, results can also be recorded in a sound form and transmitted through any suitable medium, e.g., analog or digital cable lines, fiber optic cables, etc., via telephone, facsimile, wireless mobile phone, internet phone and the like.

Thus, the information and data on a test result can be produced anywhere in the world and transmitted to a different location. As an illustrative example, when an expression level, activity level, or sequencing (or genotyping) assay is conducted outside the United States, the information and data on a test result may be generated, cast in a transmittable form as described above, and then imported into the United States. Accordingly, the present invention also encompasses a method for producing a transmittable form of information on at least one of (a) expression level or (b) activity level for at least one patient sample. The method comprises the steps of (1) determining at least one of (a) or (b) above according to methods of the present invention; and (2) embodying the result of the determining step in a transmittable form. The transmittable form is a product of such a method.

Techniques for analyzing such expression, activity, and/or sequence data (indeed any data obtained according to the invention) will often be implemented using hardware, software or a combination thereof in one or more computer systems or other processing systems capable of effectuating such analysis.

Thus, the present invention further provides a system for determining gene expression in a tumor sample, comprising: (1) a sample analyzer for determining the expression levels of a panel of genes in a sample (e.g., a tumor sample) including at least 2, 4, 6, 8 or 10 cell-cycle genes, wherein the sample analyzer contains the sample which is from a patient having lung cancer, or mRNA molecules from the patient sample or cDNA molecules from mRNA expressed from the panel of genes; (2) a first computer program for (a) receiving gene expression data on at least 4 test genes selected from the panel of genes, (b) weighting the determined expression of each of the test genes, and (c) combining the weighted expression to provide a test value, wherein at least 20%, 50%, at least 75% or at least 90% of the test genes are cell-cycle genes (or wherein the cell-cycle genes are weighted to contribute at least 50%, 60%, 70%, 80%, 90%, 95% or 100% of the test value); and (3) a second computer program for comparing the test value to one or more reference values each associated with (a) a predetermined degree of risk of cancer recurrence or progression of cancer and/or (b) a predetermined degree of likelihood of response to a particular treatment regimen (e.g., treatment regimen comprising chemotherapy). In some embodiments, the system further comprises a display module displaying the comparison between the test value to the one or more reference values, or displaying a result of the comparing step.

In some embodiments, the amount of RNA transcribed from the panel of genes including test genes is measured in the sample. In addition, the amount of RNA of one or more housekeeping genes in the sample is also measured, and used to normalize or calibrate the expression of the test genes, as described above.

In some embodiments, the plurality of test genes includes at least 2, 3 or 4 cell-cycle genes, which constitute at least 50%, 75% or 80% of the plurality of test genes, and preferably 100% of the plurality of test genes. In some embodiments, the plurality of test genes includes at least 5, 6 or 7, or at least 8 cell-cycle genes, which constitute at least 20%, 25%, 30%, 40%, 50%, 60%, 70%, 75%, 80% or 90% of the plurality of test genes, and preferably 100% of the plurality of test genes.

In some other embodiments, the plurality of test genes includes at least 8, 10, 12, 15, 20, 25 or 30 cell-cycle genes, which constitute at least 20%, 25%, 30%, 40%, 50%, 60%, 70%, 75%, 80% or 90% of the plurality of test genes, and preferably 100% of the plurality of test genes.

The sample analyzer can be any instrument useful in determining gene expression, including, e.g., a sequencing machine, a real-time PCR machine, and a microarray instrument.

The computer-based analysis function can be implemented in any suitable language and/or browsers. For example, it may be implemented with C language and preferably using object-oriented high-level programming languages such as Visual Basic, SmallTalk, C++, and the like. The application can be written to suit environments such as the Microsoft Windows™ environment including Windows™ 98, Windows™ 2000, Windows™ NT, and the like. In addition, the application can also be written for the MacIntosh™, SUN™, UNIX or LINUX environment. In addition, the functional steps can also be implemented using a universal or platform-independent programming language. Examples of such multi-platform programming languages include, but are not limited to, hypertext markup language (HTML), JAVA™, JavaScript™, Flash programming language, common gateway interface/structured query language (CGI/SQL), practical extraction report language (PERL), AppleScript™ and other system script languages, programming language/structured query language (PL/SQL), and the like. Java™- or JavaScript™-enabled browsers such as HotJava™, Microsoft™ Explorer™, or Netscape™ can be used. When active content web pages are used, they may include Java™ applets or ActiveX™ controls or other active content technologies.

The analysis function can also be embodied in computer program products and used in the systems described above or other computer- or internet-based systems. Accordingly, another aspect of the present invention relates to a computer program product comprising a computer-usable medium having computer-readable program codes or instructions embodied thereon for enabling a processor to carry out gene status analysis. These computer program instructions may be loaded onto a computer or other programmable apparatus to produce a machine, such that the instructions which execute on the computer or other programmable apparatus create means for implementing the functions or steps described above. These computer program instructions may also be stored in a computer-readable memory or medium that can direct a computer or other programmable apparatus to function in a particular manner, such that the instructions stored in the computer-readable memory or medium produce an article of manufacture including instruction means which implement the analysis. The computer program instructions may also be loaded onto a computer or other programmable apparatus to cause a series of operational steps to be performed on the computer or other programmable apparatus to produce a computer implemented process such that the instructions which execute on the computer or other programmable apparatus provide steps for implementing the functions or steps described above.

Thus one aspect of the present invention provides a system for determining whether a patient has increased likelihood of response to a particular treatment regimen. Generally speaking, the system comprises (1) computer program for receiving, storing, and/or retrieving a patient's CCG status data (e.g., expression level, activity level, variants) and optionally clinical parameter data (e.g., clinical stage); (2) computer program for querying this patient data; (3) computer program for concluding whether there is an increased likelihood of recurrence based on this patient data; and optionally (4) computer program for outputting/displaying this conclusion. In some embodiments this means for outputting the conclusion may comprise a computer program for informing a health care professional of the conclusion.

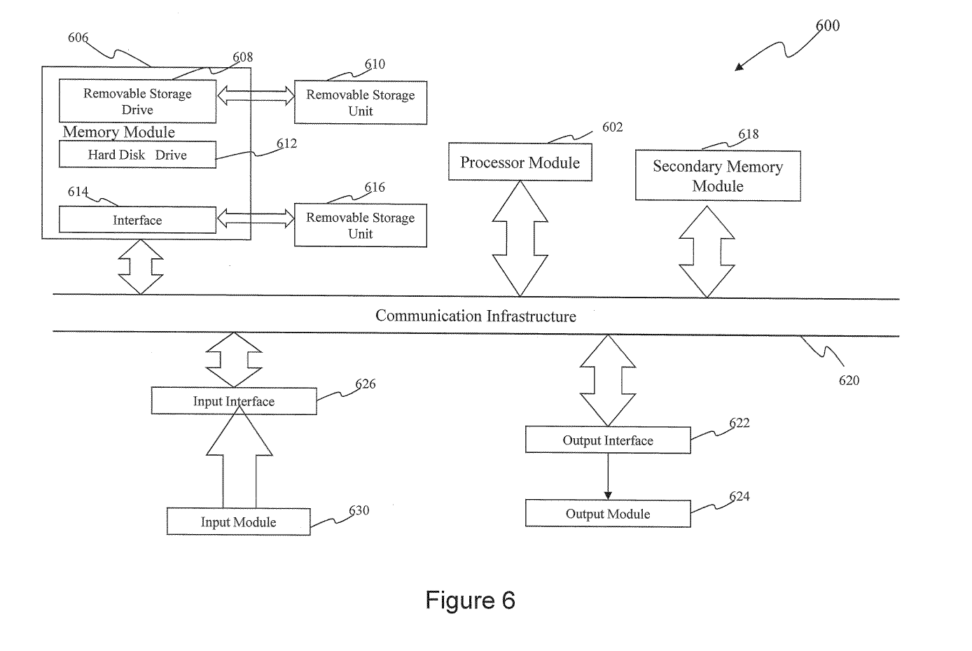

One example of such a computer system is the computer system [600] illustrated in FIG. 6. Computer system [600] may include at least one input module [630] for entering patient data into the computer system [600]. The computer system [600] may include at least one output module [624] for indicating whether a patient has an increased or decreased likelihood of response and/or indicating suggested treatments determined by the computer system [600]. Computer system [600] may include at least one memory module [606] in communication with the at least one input module [630] and the at least one output module [624].

The at least one memory module [606] may include, e.g., a removable storage drive [608], which can be in various forms, including but not limited to, a magnetic tape drive, a floppy disk drive, a VCD drive, a DVD drive, an optical disk drive, etc. The removable storage drive [608] may be compatible with a removable storage unit [610] such that it can read from and/or write to the removable storage unit [610]. Removable storage unit [610] may include a computer usable storage medium having stored therein computer-readable program codes or instructions and/or computer readable data. For example, removable storage unit [610] may store patient data. Example of removable storage unit [610] are well known in the art, including, but not limited to, floppy disks, magnetic tapes, optical disks, and the like. The at least one memory module [606] may also include a hard disk drive [612], which can be used to store computer readable program codes or instructions, and/or computer readable data.

In addition, as shown in FIG. 1, the at least one memory module [606] may further include an interface [614] and a removable storage unit [616] that is compatible with interface [614] such that software, computer readable codes or instructions can be transferred from the removable storage unit [616] into computer system [600]. Examples of interface [614] and removable storage unit [616] pairs include, e.g., removable memory chips (e.g., EPROMs or PROMs) and sockets associated therewith, program cartridges and cartridge interface, and the like. Computer system [600] may also include a secondary memory module [618], such as random access memory (RAM).

Computer system [600] may include at least one processor module [602]. It should be understood that the at least one processor module [602] may consist of any number of devices. The at least one processor module [602] may include a data processing device, such as a microprocessor or microcontroller or a central processing unit. The at least one processor module [602] may include another logic device such as a DMA (Direct Memory Access) processor, an integrated communication processor device, a custom VLSI (Very Large Scale Integration) device or an ASIC (Application Specific Integrated Circuit) device. In addition, the at least one processor module [602] may include any other type of analog or digital circuitry that is designed to perform the processing functions described herein.

As shown in FIG. 6, in computer system [600], the at least one memory module [606], the at least one processor module [602], and secondary memory module [618] are all operably linked together through communication infrastructure [620], which may be a communications bus, system board, cross-bar, etc.). Through the communication infrastructure [620], computer program codes or instructions or computer readable data can be transferred and exchanged. Input interface [626] may operably connect the at least one input module [626] to the communication infrastructure [620]. Likewise, output interface [622] may operably connect the at least one output module [624] to the communication infrastructure [620].

The at least one input module [630] may include, for example, a keyboard, mouse, touch screen, scanner, and other input devices known in the art. The at least one output module [624] may include, for example, a display screen, such as a computer monitor, TV monitor, or the touch screen of the at least one input module [630]; a printer; and audio speakers. Computer system [600] may also include, modems, communication ports, network cards such as Ethernet cards, and newly developed devices for accessing intranets or the internet.

The at least one memory module [606] may be configured for storing patient data entered via the at least one input module [630] and processed via the at least one processor module [602]. Patient data relevant to the present invention may include expression level, activity level, copy number and/or sequence information for a CCG. Patient data relevant to the present invention may also include clinical parameters relevant to the patient's disease (e.g., age, tumor size, node status, tumor stage). Any other patient data a physician might find useful in making treatment decisions/recommendations may also be entered into the system, including but not limited to age, gender, and race/ethnicity and lifestyle data such as diet information. Other possible types of patient data include symptoms currently or previously experienced, patient's history of illnesses, medications, and medical procedures.

The at least one memory module [606] may include a computer-implemented method stored therein. The at least one processor module [602] may be used to execute software or computer-readable instruction codes of the computer-implemented method. The computer-implemented method may be configured to, based upon the patient data, indicate whether the patient has an increased likelihood of recurrence, progression or response to any particular treatment, generate a list of possible treatments, etc.

In certain embodiments, the computer-implemented method may be configured to identify a patient as having or not having an increased likelihood of recurrence or progression. For example, the computer-implemented method may be configured to inform a physician that a particular patient has an increased likelihood of recurrence. Alternatively or additionally, the computer-implemented method may be configured to actually suggest a particular course of treatment based on the answers to/results for various queries.

FIG. 7 illustrates one embodiment of a computer-implemented method [700] of the invention that may be implemented with the computer system [600] of the invention. The method [700] begins with one of three queries ([710], [711]), either sequentially or substantially simultaneously. If the answer to/result for any of these queries is “Yes” [720], the method concludes [730] that the patient has an increased likelihood of recurrence or of response to a particular treatment regimen (e.g., treatment comprising chemotherapy). If the answer to/result for all of these queries is “No” [721], the method concludes [731] that the patient does not have an increased likelihood of recurrence or of response to a particular treatment regimen (e.g., treatment comprising chemotherapy). The method [700] may then proceed with more queries, make a particular treatment recommendation ([740], [741]), or simply end.

When the queries are performed sequentially, they may be made in the order suggested by FIG. 7 or in any other order. Whether subsequent queries are made can also be dependent on the results/answers for preceding queries. In some embodiments of the method illustrated in FIG. 7, for example, the method asks about clinical parameters [711] first and, if the patient has one or more clinical parameters identifying the patient as at increased likelihood of recurrence or response to a particular treatment then the method concludes such [730] or optionally confirms by querying CCG status, while if the patient has no such clinical parameters then the method proceeds to ask about CCG status [710]. As mentioned above, the preceding order of queries may be modified. In some embodiments an answer of “yes” to one query (e.g., [710]) prompts one or more of the remaining queries to confirm that the patient has increased risk of recurrence.

In some embodiments, the computer-implemented method of the invention [700] is open-ended. In other words, the apparent first step [710 and/or 711] in FIG. 7 may actually form part of a larger process and, within this larger process, need not be the first step/query. Additional steps may also be added onto the core methods discussed above. These additional steps include, but are not limited to, informing a health care professional (or the patient itself) of the conclusion reached; combining the conclusion reached by the illustrated method [700] with other facts or conclusions to reach some additional or refined conclusion regarding the patient's diagnosis, prognosis, treatment, etc.; making a recommendation for treatment (e.g., “patient should/should not undergo adjuvant chemotherapy”); additional queries about additional biomarkers, clinical parameters (e.g., age, tumor size, node status, tumor stage), or other useful patient information (e.g., age at diagnosis, general patient health, etc.).

Regarding the above computer-implemented method [700], the answers to the queries may be determined by the method instituting a search of patient data for the answer. For example, to answer the respective queries [710, 711], patient data may be searched for CCG status (e.g., CCG expression level data) and/or clinical parameters (e.g., tumor stage, nomogram score, etc.). If such a comparison has not already been performed, the method may compare these data to some reference in order to determine if the patient has an abnormal (e.g., elevated, low, negative) status. Additionally or alternatively, the method may present one or more of the queries [710, 711] to a user (e.g., a physician) of the computer system [100]. For example, the questions [710, 711] may be presented via an output module [624]. The user may then answer “Yes” or “No” or provide some other value (e.g., numerical or qualitative value incorporating or representing CCG status) via an input module [630]. The method may then proceed based upon the answer received. Likewise, the conclusions [730, 731] may be presented to a user of the computer-implemented method via an output module [624].

Thus in some embodiments the invention provides a method comprising: accessing information on a patient's CCG status stored in a computer-readable medium; querying this information to determine whether a sample obtained from the patient shows increased expression of a plurality of test genes comprising at least 2 CCGs (e.g., a test value incorporating or representing the expression of this plurality of test genes that is weighted such that CCGs contribute at least 50% to the test value, such test value being higher than some reference value); outputting [or displaying] the quantitative or qualitative (e.g., “increased”) likelihood that the patient will respond to a particular treatment regimen. As used herein in the context of computer-implemented embodiments of the invention, “displaying” means communicating any information by any sensory means. Examples include, but are not limited to, visual displays, e.g., on a computer screen or on a sheet of paper printed at the command of the computer, and auditory displays, e.g., computer generated or recorded auditory expression of a patient's genotype.

The practice of the present invention may also employ conventional biology methods, software and systems. Computer software products of the invention typically include computer readable media having computer-executable instructions for performing the logic steps of the method of the invention. Suitable computer readable medium include floppy disk, CD-ROM/DVD/DVD-ROM, hard-disk drive, flash memory, ROM/RAM, magnetic tapes and etc. Basic computational biology methods are described in, for example, Setubal et al., INTRODUCTION TO COMPUTATIONAL BIOLOGY METHODS (PWS Publishing Company, Boston, 1997); Salzberg et al. (Ed.), COMPUTATIONAL METHODS IN MOLECULAR BIOLOGY, (Elsevier, Amsterdam, 1998); Rashidi & Buehler, BIOINFORMATICS BASICS: APPLICATION IN BIOLOGICAL SCIENCE AND MEDICINE (CRC Press, London, 2000); and Ouelette & Bzevanis, BIOINFORMATICS: A PRACTICAL GUIDE FOR ANALYSIS OF GENE AND PROTEINS (Wiley & Sons, Inc., 2^{nd} ed., 2001); see also, U.S. Pat. No. 6,420,108.

The present invention may also make use of various computer program products and software for a variety of purposes, such as probe design, management of data, analysis, and instrument operation. See U.S. Pat. Nos. 5,593,839; 5,795,716; 5,733,729; 5,974,164; 6,066,454; 6,090,555; 6,185,561; 6,188,783; 6,223,127; 6,229,911 and 6,308,170. Additionally, the present invention may have embodiments that include methods for providing genetic information over networks such as the Internet as shown in U.S. Ser. No. 10/197,621 (U.S. Pub. No. 20030097222); Ser. No. 10/063,559 (U.S. Pub. No. 20020183936), Ser. No. 10/065,856 (U.S. Pub. No. 20030100995); Ser. No. 10/065,868 (U.S. Pub. No. 20030120432); Ser. No. 10/423,403 (U.S. Pub. No. 20040049354).

Techniques for analyzing such expression, activity, and/or sequence data (indeed any data obtained according to the invention) will often be implemented using hardware, software or a combination thereof in one or more computer systems or other processing systems capable of effectuating such analysis.

Thus one aspect of the present invention provides systems related to the above methods of the invention. In one embodiment the invention provides a system for determining a patient's prognosis and/or whether a patient will respond to a particular treatment regimen, comprising:
(1) a sample analyzer for determining the expression levels in a sample of a plurality of test genes including at least 2, 3, 4, 5, 6, 7, 8, 9, 10 or more CCGs (e.g., genes in Table 1, 2, 3, 5, 6, 7, 8, 9, 10 or 11; Panel A, B, C, D, E, F, G, H, J or K; or “sub-panels” of Panel F in Tables A′ to E′), wherein the sample analyzer contains the sample, RNA from the sample and expressed from the panel of genes, or DNA synthesized from said RNA;
(2) a first computer program for
   (a) receiving gene expression data on said plurality of test genes,
   (b) weighting the determined expression of each of the test genes with a predefined coefficient, and
   (c) combining the weighted expression to provide a test value, wherein the combined weight given to said at least 2, 3, 4, 5, 6, 7, 8, 9, 10 or more CCGs is at least 40% (or 50%, 60%, 70%, 80%, 90%, 95% or 100%) of the total weight given to the expression of all of said plurality of test genes; and
(3) a second computer program for comparing the test value to one or more reference values each associated with a predetermined likelihood of recurrence or progression or a predetermined likelihood of response to a particular treatment regimen.
   In some embodiments at least 20%, 50%, 75%, or 90% of said plurality of test genes are CCGs. In some embodiments the sample analyzer contains reagents for determining the expression levels in the sample of said panel of genes including at least 2, 3, 4, 5, 6, 7, 8, 9, 10 or more CCGs. In some embodiments the sample analyzer contains CCG-specific reagents as described below.

In another embodiment the invention provides a system for determining gene expression in a sample (e.g., tumor sample), comprising: (1) a sample analyzer for determining the expression levels of a panel of genes in a sample including at least 2, 3, 4, 5, 6, 7, 8, 9, 10 or more CCGs, wherein the sample analyzer contains the sample which is from a patient having lung cancer, RNA from the sample and expressed from the panel of genes, or DNA synthesized from said RNA; (2) a first computer program for (a) receiving gene expression data on at least 2, 3, 4, 5, 6, 7, 8, 9, 10 or more test genes selected from the panel of genes, (b) weighting the determined expression of each of the test genes with a predefined coefficient, and (c) combining the weighted expression to provide a test value, wherein the combined weight given to said at least 2, 3, 4, 5, 6, 7, 8, 9, 10 or more CCGs is at least 40% (or 50%, 60%, 70%, 80%, 90%, 95% or 100%) of the total weight given to the expression of all of said plurality of test genes; and (3) a second computer program for comparing the test value to one or more reference values each associated with a predetermined degree of risk of cancer recurrence or progression of the lung cancer. In some embodiments at least 20%, 50%, 75%, or 90% of said plurality of test genes are CCGs. In some embodiments the system comprises a computer program for determining the patient's prognosis and/or determining (including quantifying) the patient's degree of risk of cancer recurrence or progression based at least in part on the comparison of the test value with said one or more reference values.

In some embodiments, the system further comprises a display module displaying the comparison between the test value and the one or more reference values, or displaying a result of the comparing step, or displaying the patient's prognosis and/or degree of risk of cancer recurrence or progression.

In a preferred embodiment, the amount of RNA transcribed from the panel of genes including test genes (and/or DNA reverse transcribed therefrom) is measured in the sample. In addition, the amount of RNA of one or more housekeeping genes in the sample (and/or DNA reverse transcribed therefrom) is also measured, and used to normalize or calibrate the expression of the test genes, as described above.

In some embodiments, the plurality of test genes includes at least 2, 3 or 4 CCGs, which constitute at least 20%, 25%, 30%, 40%, 50%, 60%, 70%, 75%, 80% or 90% of the plurality of test genes, and preferably 100% of the plurality of test genes. In some embodiments, the plurality of test genes includes at least 5, 6 or 7, or at least 8 CCGs, which constitute at least 20%, 25%, 30%, 40%, 50%, 60%, 70%, 75%, 80% or 90% of the plurality of test genes, and preferably 100% of the plurality of test genes. Thus in some embodiments the plurality of test genes comprises at least some number of CCGs (e.g., at least 3, 4, 5, 6, 7, 8, 9, 10, 15, 20, 25, 30, 35, 40, 45, 50 or more CCGs) and this plurality of CCGs comprises the top 2, 3, 4, 5, 6, 7, 8, 9, 10, 11, 12, 13, 14, 15, 20, 25, 30, 35, 40 or more CCGs listed in Table 2, 3, 5, 6, 7, 12, 13, 14, 15, 16, 17, 18 or 19. In some embodiments the plurality of test genes comprises at least some number of CCGs (e.g., at least 3, 4, 5, 6, 7, 8, 9, 10, 15, 20, 25, 30, 35, 40, 45, 50 or more CCGs) and this plurality of CCGs comprises at least 1, 2, 3, 4, 5, 6, 7, 8, 9, 10, 15, or 20 of the following genes: ASPM, BIRC5, BUB1B, CCNB2, CDC2, CDC20, CDCA8, CDKN3, CENPF, DLGAP5, FOX111, KIAA0101, KIF11, KIF2C, KIF4A, MCM10, NUSAP1, PRC1, RACGAP1, and TPX2. In some embodiments the plurality of test genes comprises at least some number of CCGs (e.g., at least 3, 4, 5, 6, 7, 8, 9, 10, 15, 20, 25, 30, 35, 40, 45, 50 or more CCGs) and this plurality of CCGs comprises any one, two, three, four, five, six, seven, eight, nine, or ten or all of gene numbers 1 & 2, 1 to 3, 1 to 4, 1 to 5, 1 to 6, 1 to 7, 1 to 8, 1 to 9, or 1 to 10 of any of Table 2, 3, 5, 6, 7, 12, 13, 14, 15, 16, 17, 18 or 19. In some embodiments the plurality of test genes comprises at least some number of CCGs (e.g., at least 3, 4, 5, 6, 7, 8, 9, 10, 15, 20, 25, 30, 35, 40, 45, 50 or more CCGs) and this plurality of CCGs comprises any one, two, three, four, five, six, seven, eight, or nine or all of gene numbers 2 & 3, 2 to 4, 2 to 5, 2 to 6, 2 to 7, 2 to 8, 2 to 9, or 2 to 10 of any of Table 2, 3, 5, 6, 7, 12, 13, 14, 15, 16, 17, 18 or 19. In some embodiments the plurality of test genes comprises at least some number of CCGs (e.g., at least 3, 4, 5, 6, 7, 8, 9, 10, 15, 20, 25, 30, 35, 40, 45, 50 or more CCGs) and this plurality of CCGs comprises any one, two, three, four, five, six, seven, or eight or all of gene numbers 3 & 4, 3 to 5, 3 to 6, 3 to 7, 3 to 8, 3 to 9, or 3 to 10 of any of Table 2, 3, 5, 6, 7, 12, 13, 14, 15, 16, 17, 18 or 19. In some embodiments the plurality of test genes comprises at least some number of CCGs (e.g., at least 3, 4, 5, 6, 7, 8, 9, 10, 15, 20, 25, 30, 35, 40, 45, 50 or more CCGs) and this plurality of CCGs comprises any one, two, three, four, five, six, or seven or all of gene numbers 4 & 5, 4 to 6, 4 to 7, 4 to 8, 4 to 9, or 4 to 10 of any of Table 2, 3, 5, 6, 7, 12, 13, 14, 15, 16, 17, 18 or 19. In some embodiments the plurality of test genes comprises at least some number of CCGs (e.g., at least 3, 4, 5, 6, 7, 8, 9, 10, 15, 20, 25, 30, 35, 40, 45, 50 or more CCGs) and this plurality of CCGs comprises any one, two, three, four, five, six, seven, eight, nine, 10, 11, 12, 13, 14, or 15 or all of gene numbers 1 & 2, 1 to 3, 1 to 4, 1 to 5, 1 to 6, 1 to 7, 1 to 8, 1 to 9, 1 to 10, 1 to 11, 1 to 12, 1 to 13, 1 to 14, or 1 to 15 of any of Table 2, 3, 5, 6, 7, 12, 13, 14, 15, 16, 17, 18 or 19.

In some other embodiments, the plurality of test genes includes at least 8, 10, 12, 15, 20, 25 or 30 CCGs, which constitute at least 20%, 25%, 30%, 40%, 50%, 60%, 70%, 75%, 80% or 90% of the plurality of test genes, and preferably 100% of the plurality of test genes.

The sample analyzer can be any instrument useful in determining gene expression, including, e.g., a sequencing machine (e.g., Illumina HiSeg™, Ion Torrent PGM, ABI SOLiD™ sequencer, PacBio RS, Helicos Heliscope™, etc.), a real-time PCR machine (e.g., ABI 7900, Fluidigm BioMark™, etc.), a microarray instrument, etc.

In one aspect, the present invention provides methods of treating a cancer patient comprising obtaining CCG status information (e.g., the genes in Table 1, 2, 3, 5, 6, 7, 8, 9, 10 or 11; Panel A, B, C, D, E, F, G, H, J or K; or “sub-panels” of Panel F in Tables A′ to E′), and recommending, prescribing or administering a treatment for the cancer patient based on the CCG status. For example, the invention provides a method of treating a cancer patient comprising:

(1) determining the expression of a plurality of test genes, wherein said plurality of test genes comprises at least 4 (or 5, 6, 7, 8, 9, 10, 15, 20, 30 or more) CCGs;
(2) based at least in part on the determination in step (1), recommending, prescribing or administering either
   (a) a treatment regimen comprising chemotherapy (e.g., adjuvant chemotherapy) if the patient has increased expression of the plurality of test genes (e.g., and CCGs are weighted to contribute at least 50% to the determination of increased expression of the plurality of test genes), or
   (b) a treatment regimen not comprising chemotherapy if the patient does not have increased expression of the plurality of test genes (e.g., and CCGs are weighted to contribute at least 50% to the determination of increased expression of the plurality of test genes).

In one aspect, the invention provides compositions for use in the above methods. Such compositions include, but are not limited to, nucleic acid probes hybridizing to a CCG, including but not limited to a CCG listed in any of Table 1, 2, 3, 5, 6, 7, 8, 9, 10 or 11; Panel A, B, C, D, E, F, G, H, J or K; or “sub-panels” of Panel F in Tables A′ to E′ (or to any nucleic acids encoded thereby or complementary thereto); nucleic acid primers and primer pairs suitable for selectively amplifying all or a portion of such a CCG or any nucleic acids encoded thereby; antibodies binding immunologically to a polypeptide encoded by such a CCG; probe sets comprising a plurality of said nucleic acid probes, nucleic acid primers, antibodies, and/or polypeptides; microarrays comprising any of these; kits comprising any of these; etc. In some aspects, the invention provides computer methods, systems, software and/or modules for use in the above methods.

In some embodiments the invention provides a probe comprising an isolated oligonucleotide capable of selectively hybridizing to at least 2, 3, 4, 5, 6, 7, 8, 9, 10 or more of the genes in Table 1, 2, 3, 5, 6, 7, 8, 9, 10 or 11; Panel A, B, C, D, E, F, G, H, J or K; or “sub-panels” of Panel F in Tables A′ to E′. The terms “probe” and “oligonucleotide” (also “oligo”), when used in the context of nucleic acids, interchangeably refer to a relatively short nucleic acid fragment or sequence. The invention also provides primers useful in the methods of the invention. “Primers” are probes capable, under the right conditions and with the right companion reagents, of selectively amplifying a target nucleic acid (e.g., a target gene). In the context of nucleic acids, “probe” is used herein to encompass “primer” since primers can generally also serve as probes.

The probe can generally be of any suitable size/length. In some embodiments the probe has a length from about 8 to 200, 15 to 150, 15 to 100, 15 to 75, 15 to 60, or 20 to 55 bases in length. They can be labeled with detectable markers with any suitable detection marker including but not limited to, radioactive isotopes, fluorophores, biotin, enzymes (e.g., alkaline phosphatase), enzyme substrates, ligands and antibodies, etc. See Jablonski et al., NUCLEIC ACIDS RES. (1986) 14:6115-6128; Nguyen et al., BIOTECHNIQUES (1992) 13:116-123; Rigby et al., J. MOL. BIOL. (1977) 113:237-251. Indeed, probes may be modified in any conventional manner for various molecular biological applications. Techniques for producing and using such oligonucleotide probes are conventional in the art.

Probes according to the invention can be used in the hybridization/amplification/detection techniques discussed above. Thus, some embodiments of the invention comprise probe sets suitable for use in a microarray in detecting, amplifying and/or quantitating a plurality of CCGs. In some embodiments the probe sets have a certain proportion of their probes directed to CCGs—e.g., a probe set consisting of 10%, 20%, 30%, 40%, 50%, 55%, 60%, 65%, 70%, 75%, 80%, 85%, 90%, 95%, 96%, 97%, 98%, 99%, or 100% probes specific for CCGs. In some embodiments the probe set comprises probes directed to at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 40, 45, 50, 60, 70, 80, 90, 100, 125, 150, 175, 200, 250, 300, 350, 400, 450, 500, 600, 700, or 800 or more, or all, of the genes in Table 1, 2, 3, 5, 6, 7, 8, 9, 10 or 11; Panel A, B, C, D, E, F, G, H, J or K; or “sub-panels” of Panel F in Tables A′ to E′. Such probe sets can be incorporated into high-density arrays comprising 5,000, 10,000, 20,000, 50,000, 100,000, 200,000, 300,000, 400,000, 500,000, 600,000, 700,000, 800,000, 900,000, or 1,000,000 or more different probes. In other embodiments the probe sets comprise primers (e.g., primer pairs) for amplifying nucleic acids comprising at least a portion of one or more of the CCGs in Table 1, 2, 3, 5, 6, 7, 8, 9, 10 or 11; Panel A, B, C, D, E, F, G, H, J or K; or “sub-panels” of Panel F in Tables A′ to E′.

In another aspect of the present invention, a kit is provided for practicing the prognosis of the present invention. The kit may include a carrier for the various components of the kit. The carrier can be a container or support, in the form of, e.g., bag, box, tube, rack, and is optionally compartmentalized. The carrier may define an enclosed confinement for safety purposes during shipment and storage. The kit includes various components useful in determining the status of one or more CCGs and one or more housekeeping gene markers, using the above-discussed detection techniques. For example, the kit many include oligonucleotides specifically hybridizing under high stringency to mRNA or cDNA of the genes in Table 1, 2, 3, 5, 6, 7, 8, 9, 10 or 11; Panel A, B, C, D, E, F, G, H, J or K; or “sub-panels” of Panel F in Tables A′ to E′. Such oligonucleotides can be used as PCR primers in RT-PCR reactions, or hybridization probes. In some embodiments the kit comprises reagents (e.g., probes, primers, and or antibodies) for determining the expression level of a panel of genes, where said panel comprises at least 25%, 30%, 40%, 50%, 60%, 75%, 80%, 90%, 95%, 99%, or 100% CCGs (e.g., CCGs in Table 1, 2, 3, 5, 6, 7, 8, 9, 10 or 11; Panel A, B, C, D, E, F, G, H, J or K; or “sub-panels” of Panel F in Tables A′ to E′). In some embodiments the kit consists of reagents (e.g., probes, primers, and or antibodies) for determining the expression level of no more than 2500 genes, wherein at least 5, 10, 15, 20, 30, 40, 50, 60, 70, 80, 90, 100, 120, 150, 200, 250, or more of these genes are CCGs (e.g., CCGs in Table 1, 2, 3, 5, 6, 7, 8, 9, 10 or 11; Panel A, B, C, D, E, F, G, H, J or K; or “sub-panels” of Panel F in Tables A′ to E′).

The oligonucleotides in the detection kit can be labeled with any suitable detection marker including but not limited to, radioactive isotopes, fluorephores, biotin, enzymes (e.g., alkaline phosphatase), enzyme substrates, ligands and antibodies, etc. See Jablonski et al., Nucleic Acids Res., 14:6115-6128 (1986); Nguyen et al., Biotechniques, 13:116-123 (1992); Rigby et al., J. Mol. Biol., 113:237-251 (1977). Alternatively, the oligonucleotides included in the kit are not labeled, and instead, one or more markers are provided in the kit so that users may label the oligonucleotides at the time of use.

In another embodiment of the invention, the detection kit contains one or more antibodies selectively immunoreactive with one or more proteins encoded by one or more CCGs or optionally any additional markers. Examples include antibodies that bind immunologically to a protein encoded by a gene in Table 1, 2, 3, 5, 6, 7, 8, 9, 10 or 11; Panel A, B, C, D, E, F, G, H, J or K; or “sub-panels” of Panel F in Tables A′ to E′. Methods for producing and using such antibodies are well-known in the art.

Various other components useful in the detection techniques may also be included in the detection kit of this invention. Examples of such components include, but are not limited to, Taq polymerase, deoxyribonucleotides, dideoxyribonucleotides, other primers suitable for the amplification of a target DNA sequence, RNase A, and the like. In addition, the detection kit preferably includes instructions on using the kit for practice the prognosis method of the present invention using human samples. In one embodiment of the invention the CCG score is calculated from RNA expression of 31 CCGs normalized by 15 housekeeper genes (HK). The relative numbers of CCGs and HK genes are optimized in order to minimize the variance of the CCG score. The CCG score is the unweighted mean of CT values for CCG expression, normalized by the unweighted mean of the HK genes so that higher values indicate higher expression. In some embodiments, one unit is equivalent to a two-fold change in expression. In some embodiments, the CCG scores are centered by the mean value, determined in a training set.

In some embodiments, a dilution experiment is performed on commercial prostate samples to estimate the measurement error of the CCG score (se=0.10) and the effect of missing values. In some embodiments, the CCG score may remain stable as concentration decreased to the point of 10 failures out of a total 31 CCGs. In some embodiments, samples with more than 9 missing values are not assigned a CCG score.

In some embodiments, samples may be obtained from an FFPE sample block. In some embodiments, 5 μm sections may be cut from the sample block. In some embodiments sections may be stained with haematoxylin and eosin (H&E). In some embodiments, tumor areas may be marked by a pathologist. In some embodiments 10 μm sections are cut adjacent to the H&E stained sections. In some embodiments tumor areas on the unstained sections are identified by alignment with the marked areas on the H&E stain. In some embodiments tumor areas are macro-dissected manually. In some embodiments, samples are deparaffinized by xylene extractions followed by washes with ethanol. In some embodiments samples are treated overnight with proteinase K. In some embodiments samples are subjected to RNA extraction. In some embodiments, RNA extraction is performed using the Qiagen miRNAeasy kit. In some embodiments RNA is treated with DNASE I to remove potential genomic DNA contamination. In some embodiments, RNA is converted to cDNA and synthesized cDNA serves as template for replicate pre-amplification reactions. In some embodiments, samples are run on Taqman™ low density arrays (TLDA, Applied Biosystems).

In some embodiments raw data for the calculation of the CCP score equals the Cₜ values of the genes from the TLDA arrays. In some embodiments, the CCP score is the unweighted mean of Cₜ values for cell cycle gene expression, normalized by the unweighted mean of the house keeper genes so that higher values indicate higher expression. In some embodiments CCP scores are centered by the mean value determined in a commercial training set.

In one embodiment of the invention early stage lung adenocarcinoma samples can be used as a “training” cohort for the purpose of defining centering constants in lung tissue. In some embodiments these constants can be used to center the triplicate expression mean of CCP genes before averaging into CCP scores. In some embodiments distribution of CCP scores in the training cohort is similar to the distribution in any of the clinical sample sets.

In one embodiment of the invention patient samples with early stage lung adenocarcinoma may be studied. In some embodiments patients may be selected using staging criteria following the 6^{th} edition of the IASLC staging guidelines. In some embodiments other clinical data including, gender, ethnicity, smoking status, recurrence and vital status may be collected.

In one embodiment, survival data for the cohort includes disease-free survival (DFS, time from surgery to first recurrence or last follow-up for recurrence) and overall survival (OS, time from surgery to death or last follow-up for survival). In some embodiments deaths without recurrence are censored at time of death and not included as cancer-related death events.

In some embodiments, a cohort may be analyzed by Cox proportional hazard analysis using disease survival as the outcome variable. In some embodiments, continuous variables include CCP score and clinical parameters including stage (numerical, 1A=1, 1B=2, IIa=3, IIB=4), adjuvant treatment (categorical, y/n), age in years, smoking status (numerical, never=1, former=2, current=3) and gender (male/female). In some embodiments an interaction term for adjuvant treatment and stage may be introduced to account for the known difference in treatment outcome in stage IA versus other stages. In some embodiments, the test statistic for the prognostic value of the CCP score is the likelihood ratio for the full model (all clinical variable plus the CCP score) versus the reduced model (all clinical variables, no CCP score).

In some embodiments, a univariate analysis may show

that stage, CCP score and gender are significantly correlated with disease survival. In some embodiments the p-value for stage may be equal to or less than 0.05. In some embodiments the p-value for stage may be equal to or less than 0.01. In some embodiments the p-value for stage may be equal to or less than 0.00. In some embodimnets the p-value for stage may be equal to or less than 0.0001. In some embodiments the p-value may be equal to or less than 0.00045. In some embodiments the p-value for CCP score may be equal to or less than 0.05, in some embodiments the p-value for CCP score may be equal to or less than 0.01. In some embodiments the p-value for CCP score may be equal to or less than 0.0013 or less. In some embodiments the p-value for gender may be equal to or less than 0.05, in some embodiments the p-value for stage may be equal to or less than 0.054.

In some embodiments, a multivariate analysis may show that CCP score is a significant predictor of disease survival when added to a model of all clinical parameters. In some embodiments the CCP score may be equal to or less than 0.05. In some embodiments the CCP score may be equal to or less than 0.0175. In some embodiments the Hazard Ratio may be equal to or greater than 1.52. In some embodiments, the 95% confidence interval may be equal to 1.04 and 2.24. In some embodiments the lowest CCP quartile has a 5-year survival expectation of 98%, In some embodiments the highest CCP quartile has a 5-year survival rate of 60%.

In some embodiments stage I and stage II patients partition across all four CCP quartiles. Thus, in some embodiments CCP score can be used to modify treatment considerations depending on risk estimates besides clinical staging criteria.

In some embodiments stage IB samples may be analyzed separately. In some embodiments CCP score is a significant predictor of outcome for stage IB patients. In some embodiments the CCP score p-value is equal to or less than 0.05. In some embodiments the CCP score p-value is equal to or less than 0.02. In some embodiments CCP score may be used as a threshold for a high risk (above the mean) and low risk groups (below the mean). In some embodiments the low risk group may have a survival rate of 95% or higher. In some embodiments the high risk group may have a survival rate of 75% or lower. In some embodiments stage IB samples in the highest CCP quartile have a 5-year survival rate of 80% or higher. In some embodiments, stage IB samples in the lowest CCP quartile have a 5-year survival rate of 30% or lower.

In some embodiments, the CCP score not only acts as a prognostic (by identifying rapidly progressing cancers) but may also be indicative of treatment benefit (by identifying cancers that will be most susceptible to disruption of the cell cycle.). In some embodiments the test statistic is the likelihood ratio for the full model (all clinical variable, CCP score and CCP:adjuvant treatment interaction term) versus the reduced model (all clinical variables no CCP score, no interaction term). In some embodiments, the interaction for CCP score and adjuvant treatment is not formally significant at the 0.05 level. In some embodiments, the interaction for CCP score is equal to or less than 0.07. In some embodiments untreated patients in the highest CCP quartile have a survival rate of 30% or lower. In some embodiments untreated patients in the lowest CCP quartile have survival rates of 70% or higher. In some embodiments patients treated with adjuvant therapy in the highest CCP quartile have a survival rate of 70% or higher. In some embodiments a high CCP score correlates strongly with a higher likelihood of response to adjuvant chemotherapy.

In another aspect of the invention, the prognostic value of CCP in terms of p-values and standardized hazard ratios from univariate, and multivariate, Cox proportional hazards models is evaluated. In some embodiments, the endpoint may be death from disease within five years of surgery. In some embodiments death from disease can be defined as death following recurrence. In some embodiments patients who are lost to follow-up or died of other causes are censored from the analysis.

In some embodiments univariate p-values are based on the partial likelihood ratio. In some embodiments multivariate p-values are based on the partial likelihood ratio for the change in deviance from a full model versus a reduced model. In some embodiments the full model includes all relevant covariates. In some embodiments the reduced model includes all covariates except for the covariate being evaluated, and any interaction terms involving the covariate being evaluated. In some embodiments hazard ratios are standardized to represent the increased risk associated with a one standard deviation increase in CCP score.

In some embodiments CCP score may be combined with clinical variables in multivariate Cox proportional hazards models. In some embodiments clinical data for age, gender, smoking status, stage, adjuvant treatment, pleural invasion, and/or tumor size is included. In some embodiments an interaction term for stage with treatment is included.

In some embodiments categorical clinical variables are coded to explain the maximum possible variability in patient outcomes. In some embodiments stage may be coded as a 4-level categorical variable (IA, IB, IIA, IIB) rather than a 2-level categorical variable (I,II). In some embodiments less significant p-values may be associated with stage.

In some embodiments the appropriateness of combining cohorts may be assessed. In some embodiments Cox proportional hazards models may be constructed for each of the clinical variables, consisting of the clinical variable in question, a variable designating cohort, and an interaction term. In some embodiments, interaction terms may have a p-value greater than 0.05 in two-sided likelihood ratio tests.

In some embodiments the appropriateness of the proportional hazards assumption may be evaluated. In some embodiments, time dependence for the hazard ratio of the CCP score is not supported. In some embodiments the possibility that CCP score might have a non-linear effect is evaluated. In some embodiments second- and third-order polynomials for CCP score are tested in Cox proportional hazards models but were not significant at the 5% level.

In some embodiments a Cox proportional hazards models is constructed for each available clinical variable, consisting of the clinical variable in question, CCP score, and an interaction term. In some embodiments the p-value for the interaction terms is greater than 0.05.

In some embodiments variables for each patient include age, gender, smoking status, stage, adjuvant treatment, tumor size, pleural invasion, cohort, and/or CCP score. In some embodiments age in years is a quantitative variable. In some embodiments gender is a binary variable (male, female). In some embodiments, smoking status is a 3-level categorical variable (never, former, current). In some embodiments pathological stage is according to the 7th edition TNM classification. In some embodiments pathological stage is a 4-level categorical variable (IA, IB, IIA, IIB). In some embodiments adjuvant treatment is a binary variable (no, yes). In some embodiment tumor size is a quantitative variable. In some embodiments tumor size is measured in centimeters. In some embodiments pleural invasion is a binary variable (no, yes). In some embodiments cohort is a 2-level categorical variable. In some embodiments CCP score is a quantitative variable.

In some embodiments univariate analysis assess CCP scores ability to predict five year survival. In some embodiments the p-value is equal to or less than 0.05. In some embodiments the p-value is equal to or less than 0.01. In some embodiments the p-value is equal to or less than 0.001. In some embodiments the p-value is equal to or less than 0.0003. In some embodiments multivariate analysis assesses CCP's ability to predict five-year survival. In some embodiments the p-value is equal to or less than 0.05. In some embodiments the p-value is equal to or less than 0.01. In some embodiments the p-value is equal to or less than 0.007. In some embodiments the standardized Hazard Ratio is equal to 1.50. In some embodiments the 95% Confidence Intervals are equal to 1.11 and 2.02. In some embodiments the results from multivariate analysis indicate that the CCP score is able to capture a significant amount of prognostic information independent of the many clinical variables. In some embodiments 5-year disease survival for patients with low CCP scores is 92% or higher. In some embodiments 5-year disease survival for patients with medium CCP scores is 79% in patients or lower. In some embodiments 5-year disease survival for patients with high CCP scores is 73% or lower.

In another aspect of the invention the relationship between CCP score and absolute benefit from adjuvant treatment is analyzed. In some embodiments CCP score maybe be used to predict survival in patients treated with adjuvant therapies.

In some embodiments the technique of Zhang & Klein (Confidence bands for the difference of two survival curves under the proportional hazards model, LIFETIME DATA ANALYSIS (2001)7:243-254) may be used to evaluate the absolute difference in 5-year predicted risk of disease-related death for patients who received adjuvant treatment versus patients who did not receive adjuvant treatment over a range of observed CCP scores. In some embodiments complex contrast coding may be used to test whether the absolute difference, due to treatment, in the hazard of disease related death is greater for patients with high CCP scores than for patients with low CCP scores.

In some embodiments the Zhang & Klein method may be used to test for differences in survival between two treatments (or between patients receiving treatment, and patients not receiving treatment) after adjusting for the effects of other covariates. In some embodiments estimates of absolute treatment benefit may be calculated together with point wise confidence bands, over a range of observed CCP scores.

In some embodiments contrast coding may be used as to test whether the absolute decrease in the hazard of disease-related death due to adjuvant treatment is significantly greater for patients with high CCP scores than for patients with low CCP scores. In some embodiments CCP scores may be categorized as high or low using the median as the cutoff point. In some embodiments each patient may be assigned to one of four groups: high CCP with adjuvant treatment (ht), high CCP without adjuvant treatment (hu), low CCP with adjuvant treatment (lt), and low CCP without adjuvant treatment (lu). In some embodiments, the null hypothesis is H₀: ht−hu=lt−lu. In some embodiments the null hypothesis is H₀: ht−hu−lt+lu=0. In some embodiments the null hypothesis may be tested with Cox proportional hazards regression, using 5-year disease related death as the outcome, by applying the complex contrast vector c=(1, −1, −1, 1). In some embodiments significantly greater absolute treatment benefit is indicated for patients with high CCP scores compared to patients with low CCP scores. In some embodiments the p-value is equal to or lower than 0.05. In some embodiments the p-value is equal to or lower than 0.01. In some embodiments the p-value is equal to or lower than 0.0060. In some embodiments the association between CCP score and absolute treatment benefit maintains significance after adjusting for age, gender, smoking status, stage, tumor size, and pleural invasion status in the complex contrast model. In some embodiments the p-value is equal to or lower than 0.05. In some embodiments the p-value is equal to or lower than 0.024).

In another aspect of the invention, a combined prognostic score of pathological stage (pStage) and the CCP expression score may be modeled in stage I and II patients without adjuvant treatment. In some embodiments DC values may be centered by processing site and scaled by the ratio of the standard deviations of the CCP score in qPCR and microarray data. In some embodiments the outcome measure is five year disease-specific survival. In some embodiments coefficients for the combination of CCP and pStage are derived from a bivariate Cox proportional hazards model. In some embodiments pathological stage is modeled as numerical variable (IA=1, IB=2, IIA=3, IIB=4). In some embodiments the Cox PH model may be stratified by cohort. In some embodiments cohorts are evaluated individually. In some embodiments coefficients for a final model may be derived from a combination of all cohorts. In some embodiments the final prognostic score may be scaled to represent values between 0 and 80.

In some embodiments hazard ratios for CCP score and pathological stage are consistent across the various cohorts. In some embodiments CCP together with pathological stage provides the best prediction for lung cancer mortality. In some embodiments Prognostic score=20*(0.33*CCP score+0.52*stage)+15. In some embodiments the p-value is equal to or less than 0.05. In some embodiments the p-value is equal to or less than 0.01. In some embodiments the p-value is equal to or less than 0.001. In some embodiments the p-value is equal to or less than 0.00078.

In some embodiments the combined score may differentiate 5-year lung cancer mortality risk for patients assigned the same risk based on pathological stage alone. In some embodiments pathological stage alone may provided estimates of 5-year risk of cancer-specific death. In some embodiments stage IA provides a 5-year risk of cancer-specific death estimate of 12.6% or less. In some embodiments stage IB provides a 5-year risk of cancer-specific death estimate of 22.6% or less. In some embodiments stage HA provides a 5-year risk of cancer-specific death estimate of 38.4% or more. In some embodiments stage IIB provides a 5-year risk of cancer-specific death estimate of 60% or more. In some embodiments the prognostic score may be used to separate stage IA patients with 5-year risk estimates ranging from 6% to 24%. In some embodiments the prognostic score may be used to separate stage IB patients with 5-year risk estimates ranging from 10% to 42%. In some embodiments the prognostic score may be used to separate stage IIA patients with 5-year risk estimates ranging from 21% to 63%. In some embodiments the prognostic score may be used to separate stage IIB patients with 5-year risk estimates ranging from 32% to 75%.

In some embodiments a pre-defined prognostic score (PS) is calculated for each patient. In some embodiments a PS cut-point is determined such that the percentage of stage IA patients having a PS at or below the cutpoint is close as possible to 85%.

In some embodiments the association of CCP, and the PS, with 5-year lung cancer mortality is evaluated using Cox proportional hazards models, likelihood ratio tests or both. In some embodiments the Mantel-Cox logrank test is used to evaluate the difference in 5-year lung cancer mortality for patients with PS scores at or below a cut-point versus patients with scores above a cut-point.

In some embodiments PS may be used to predict 5 year lung cancer specific survival. In some embodiments low and high risk may be classified by a cut-off predefined as the 85% percentile of the PS in stage IA patients. In some embodiments there is a significant difference between the average risk between low and high risk patient groups.

In some embodiments patients in the low PS group have a significantly more favorable 5-year survival than patients in the high PS group. In some embodiments the Log-rank p value is at least 3.8×10⁻⁷.

In some embodiments risk stratification is improved by PS compared to pathological stage alone. In some embodiments patients with pathological stage lA have an 18% risk of disease specific death within five years. In some embodiments patients with pathological stage IB have a 28% risk of disease specific death within five years. In some embodiments patients with pathological stage IIA have a 42% risk of disease specific death within five years. In some embodiments patients with pathological stage IIB have a 60% risk of disease specific death within five years. In some embodiments, pathological stage is combined with CCP score resulting in the ability to assigned significantly more detailed risk to patients assigned identical risk according to pathological stage alone.

In some embodiments CCP score alone is a significant prognostic marker. In some embodiments CCP score is evaluated using univariate analysis. In some the univariate p-value is at least 0.05. In some the univariate p-value is at least 0.01. In some the univariate p-value is at least 0.001. In some the univariate p-value is at least 0.0001. In some the univariate p-value is at least 0.00001. In some the univariate p-value is at least 0.0000011. In some embodiments CCP score is evaluated using multivariate analysis. In some embodiments CCP score is evaluated using multivariate analysis. In some the multivariate p-value is at least 0.05. In some the multivariate p-value is at least 0.01. In some the multivariate p-value is at least 0.005.

In some embodiments the prognostic value of PS is evaluated by univariate analysis. In some embodiments the p-value is at least 0.05. In some embodiments the p-value is at least 0.01. In some embodiment the p-value is at least 0.001. In some embodiments the p-value is at least 2.8×10̂−11. In some embodiments the prognostic value of PS is evaluated by bivariate analysis. In some embodiments the p-value is at least 0.05. In some embodiments the p-value is at least 0.01. In some embodiments the p-value is at least 0.093. In some embodiments the combination of pathological stage and CCP score into the Prognostic Score captures significant prognostic information that is not provided by pathological stage alone.

In some embodiments the prognostic value of the PS is evaluated in IA and IB stage cancer separately using a univariate model. In some embodiments the Hazard Ratio is 1.67. In some embodiments the 95% confidence intervals are 1.27, and 2.29. In some embodiments the p-value is at least 0.05. In some embodiments the p-value is at least 0.01. In some embodiments the p-value is at least 0.001. In some embodiments the p-value is at least 0.0027. In some embodiments the prognostic value of the PS is evaluated in IA and IB stage cancer separately using a bivariate model. In some embodiments the Hazard Ratio is 1.74. In some embodiments the 95% confidence intervals are 1.16, and 2.61. In some embodiments the p-value is at least 0.05. In some embodiments the combination of pathological stage and CCP score into the Prognostic Score captures significant prognostic information that is not provided by pathological stage alone when restricted to stage IA-IB disease.

In another embodiment of the invention CCP expression and pathological stage may be used to assess prognosis for post-surgical risk of death in patients diagnosed with lung carcinoids.

In some embodiments, CCP scores may be generated stage IA, IB, IIA, IIB, and IIIB lung carcinoid patients. In some embodiments the outcome measure is survival.

In some embodiments the association of CCP with mortality is evaluated using the Cox proportional hazards model. In some embodiments the p-value in a univariate analysis is at least 0.05. In some embodiments the p-value in a univariate analysis is at least 0.01. In some embodiments the p-value in a univariate analysis is at least 0.00125. In some embodiments the p-value in a multivariate analysis is at least 0.05. In some embodiments the p-value in a multivariate analysis is at least 0.01. In some embodiments the p-value in a multivariate analysis is at least 0.0035.

In another embodiment of the invention CCP expression and pathological stage may be used to assess prognosis for post-surgical risk of death in patients diagnosed with lung carcinoids.

In some embodiments disease may be spread among two histological groups: atypical and typical. In some embodiments stage may be coded as a 4-level categorical variable. In some embodiments stages may consist of IA, IB, IIA/IIB, and IIIA/IIIB/IV.

In some embodiments the association of CCP with death from disease may be evaluated using the Cox proportional hazards model. In some embodiments univariate analysis of Cox proportional hazards models may be used to evaluate the association of CCP with death from lung carcinoids. In some embodiments the p-value is at least 0.05. In some embodiments the p-value is at least 0.01. In some embodiments the p-value is at least 0.0014. In some embodiments the association of CCP with disease free survival may be evaluated using the Cox proportional hazards model. In some embodiments univariate analysis of Cox proportional hazards models may be used to evaluate the association of CCP with disease free survival. In some embodiments the p-value is at least 0.05. In some embodiments the p-value is at least 0.01. In some embodiments the p-value is at least 0.006.

In some embodiments the association of CCP and death with disease in atypical carcinoid patients may be evaluated using the Cox proportional hazards model. In some embodiments univariate analysis may be used to evaluate the association of CCP and death with disease in atypical carcinoid patients. In some embodiments CCP is a highly significant predictor of death with recurrence of disease. In some embodiments the p-value is at least 0.05. In some embodiment the p-value is at least 0.0102.

### Example 1

The expression profile described here as a prognostic and predictive tool in NSCLC adenocarcinoma was composed of 31 CCP genes (Panel F) and 15 housekeeping genes (Table A) used to normalize RNA content per sample. The gene panel is further described in International Application No. PCT/US2010/020397 (pub. no. WO/2010/080933).

### CCG Score

The CCG score was calculated from RNA expression of 31 CCGs (Panel F) normalized by 15 housekeeper genes (HK). The relative numbers of CCGs (31) and HK genes (15) were optimized in order to minimize the variance of the CCG score. The CCG score is the unweighted mean of CT values for CCG expression, normalized by the unweighted mean of the HK genes so that higher values indicate higher expression. One unit is equivalent to a two-fold change in expression. The CCG scores were centered by the mean value, again determined in the training set.

A dilution experiment was performed on four of the commercial prostate samples to estimate the measurement error of the CCG score (se=0.10) and the effect of missing values. It was found that the CCG score remained stable as concentration decreased to the point of 10 failures out of the total 31 CCGs. Based on this result, samples with more than 9 missing values were not assigned a CCG score.

### Experimental Procedures

From each FFPE sample block one 5 μm section was cut and stained with haematoxylin and eosin. Tumor areas were marked by a pathologist. Additional two 10 μm sections were cut directly adjacent to the H&E stained section. Tumor areas on the unstained sections were identified by alignment with the marked areas on the H&E stain and macro-dissected manually into Eppendorff tubes. Sections were deparaffinized by xylene extractions followed by washes with ethanol. After an overnight incubation with proteinase K, deparaffinized tissue was subjected to RNA extraction using the Qiagen miRNAeasy kit according to manufacturer's instructions. Total RNA was treated with DNASE I to remove potential genomic DNA contamination. Final RNA yield was determined on a Nanodrop spectrophotometer.

For each sample 500 ng RNA was converted to cDNA using the high capacity cDNA archive kit (Applied Biosystems). Newly synthesized cDNA served as template for replicate pre-amplification reactions. Each of the reactions contained 3 μl cDNA and a pool of Taqman™ assays for all 46 genes in the signature (15 housekeeping genes, 31 cell cycle genes). Preamplification was run for 14 cycles to generate sufficient total copies even from a low copy sample to inoculate individual PCR reactions for 46 genes. Preamplification reactions were diluted 1:20 before loading on Taqman™ low density arrays (TLDA, Applied Biosystems). Raw data for the calculation of the CCP score were the Cₜ values of the 46 genes from the TLDA arrays. The CCP score was the unweighted mean of Cₜ values for cell cycle gene expression, normalized by the unweighted mean of the house keeper genes so that higher values indicate higher expression. One unit is equivalent to a two-fold change in expression. The CCP scores were centered by the mean value determined in the commercial training set.

### Commercial Samples

Early stage (IA, IB, IIA, IIB) lung adenocarcinoma samples were purchased from two sources. This sample set was considered the “training” cohort for the purpose of defining centering constants in lung tissue. These constants were used to center the triplicate expression mean of CCP genes before averaging into CCP scores. This avoided giving undue influence of outlier genes when calculating the CCP gene average. CCP scores were ascertained as described bove. Distribution of CCP scores in this training cohort was similar to the distribution in any of the clinical sample sets.

### Clinical Sample Set 1

A total of 200 patient samples with early stage lung adenocarcinoma was used in this study. These patients were selected from a cohort ascertained between 1995 and 2001. Staging criteria were following the 6^{th} edition of the IASLC staging guidelines. Clinical parameters of the cohort are summarized in Table B.

TABLE B

| Variable | | N |
|---|---|---|
| Gender | Male | 96 |
| | Female | 104 |
| Ethnicity | Caucasian | 178 |
| | Non- | 22 |
| | Caucasian | |
| Smoking | Never | 28 |
| status | smoker | |
| | Former | 81 |
| | Smoker | |
| | Current | 91 |
| | Smoker | |
| Recurrence | No | 119 |
| | Yes | 71 |
| | Unknown | 9 |
| Vital Status | Alive | 113 |
| | Deceased | 87 |

CCP scores for 199 samples were generated as described above. One sample did not contain tumor. 38 samples were of advanced stage (IIIA, IIB, IV) and were excluded from analysis. Two samples had undefined metastasis status (Mx) and were removed for analysis purposes. 32 patients had received neoadjuvant treatment. Since this may affect staging and prior staging was not available, neoadjuvant treated samples were omitted from analysis. Four samples were excluded for synchronous cancers and one patient sample was duplicate. For the final analysis 137 stage I and stage II samples remained (see Table C).

TABLE C

| | | Eligible |
|---|---|---|
| | | for |
| | N | analysis |
| Samples | 200 | 200 |

| | | | |
|---|---|---|---|
| Stage | IA + IB | 129 | 162 |
| | IIA + IIB | 33 | |
| | IIIA + IIIB + III | 30 | |
| | IV | 8 | |
| M stage | Mx | 2 | 160 |
| Neoadjuvant | No | 168 | 144 |
| | Yes | 32 | |
| Adjuvant | No | 141 | 142 |
| | Yes | 50 | |
| | Unknown | 9 | |
| | | 4 | 139 |

Synchronous other cancer

| | | | |
|---|---|---|---|
| Tumor | Negative | 1 | 138 |
| content | | | |

| | | |
|---|---|---|
| Duplicate patient | 1 | 137 |

Survival data for the cohort included disease-free survival (DFS, time from surgery to first recurrence or last follow-up for recurrence) and overall survival (OS, time from surgery to death or last follow-up for survival). A total of 45 recurrences and 50 deaths were observed in the 137 samples included in the analysis. However, only 32 deaths were preceded by a recurrence suggesting that a large number of death events were not related to disease. Deaths without recurrence were censored at time of death and not included as cancer-related death events. The “death with recurrence” outcome measure is referred to as DS (disease survival).

The cohort was analysed by Cox proportional hazard analysis using DS as outcome variable. Besides the CCP score as continuous variable, clinical parameters in the models included stage (numerical, 1A=1, 1B=2, IIa=3, IIB=4), adjuvant treatment (categorical, y/n), age in years, smoking status (numerical, never=1, former=2, current=3) and gender (male/female). In addition, an interaction term for adjuvant treatment and stage was introduced to account for the known difference in treatment outcome in stage IA vs. the remaining stages. The test statistic for the prognostic value of the CCP score is the likelihood ratio for the full model (all clinical variable plus the CCP score) versus the reduced model (all clinical variables, no CCP score).

In univariate analysis, only stage (p=0.000045), CCP score (p=0.0013) and gender (p=0.054) were significantly correlated with disease survival (see Table D).

TABLE D

Variable

| | Univariate | Multivariate |
|---|---|---|
| | (Disease | (Disease |
| | Survival) | Survival) |
| Stage | 4.6 × 10⁻⁵ | |
| CCP | 0.0013 | 0.0175 (HR |
| | | 1.52; 95% CI |
| | | 1.04, 2.24) |
| Gender | 0.054 | |
| Age | 0.22 | |
| Smoking | 0.93 | |
| Treatment | 0.8 | |

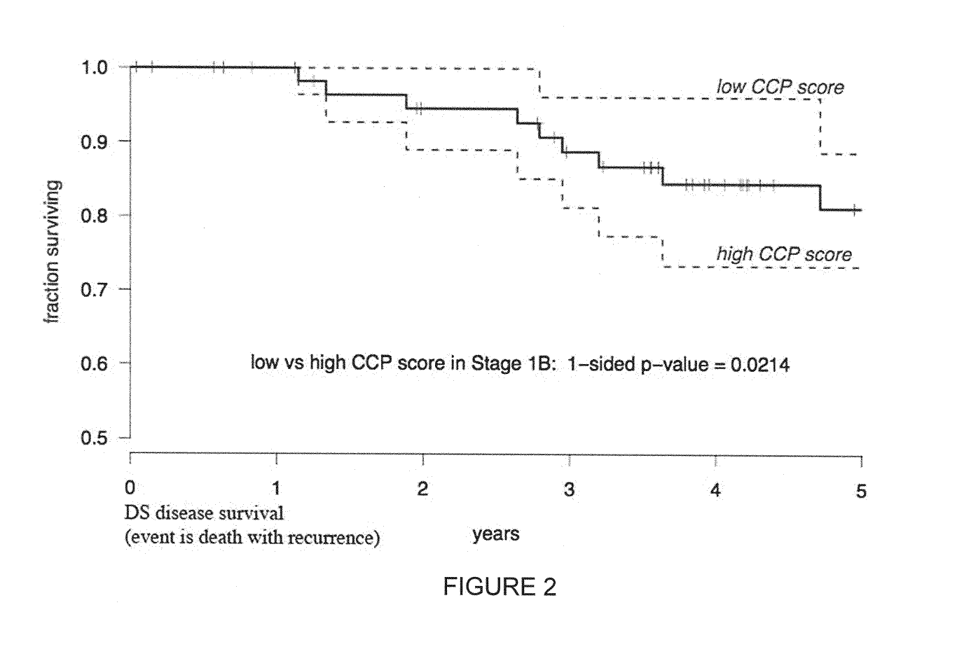

In multivariate analysis, CCP score remained a significant predictor of disease survival when added to a model of all clinical parameters (p=0.0175, HR 1.52, 95% CI 1.04, 2.24). A Kaplan-Meier analysis for the stage I and II cohort using CCP score quartiles is shown in FIG. 2. The lowest CCP quartile has a 5-year survival expectation of 98%, the highest CCP quartile has a 5-year survival rate of 60%. The stage distribution within the CCP quartiles is shown in Table E.

TABLE E

| CCP | | Stage | | Stage | 5-year |
|---|---|---|---|---|---|
| Score | Stage I | II | Stage I | II | Survival |
| Quartile | (N) | (N) | (%) | (%) | (%) |
| 1 | 31 | 2 | 30 | 8 | 98 |
| 2 | 27 | 5 | 26 | 19 | 78 |
| 3 | 24 | 8 | 23 | 31 | 76 |
| 4 | 21 | 11 | 20 | 42 | 60 |

Both stage I and stage II patients partition across all four CCP quartiles, supporting the assumption that patients of high risk exist within the lowest stage group and patients with reduced risk can be found among higher stages. Thus, the CCP score can be used to modify treatment considerations depending on risk estimates besides clinical staging criteria.

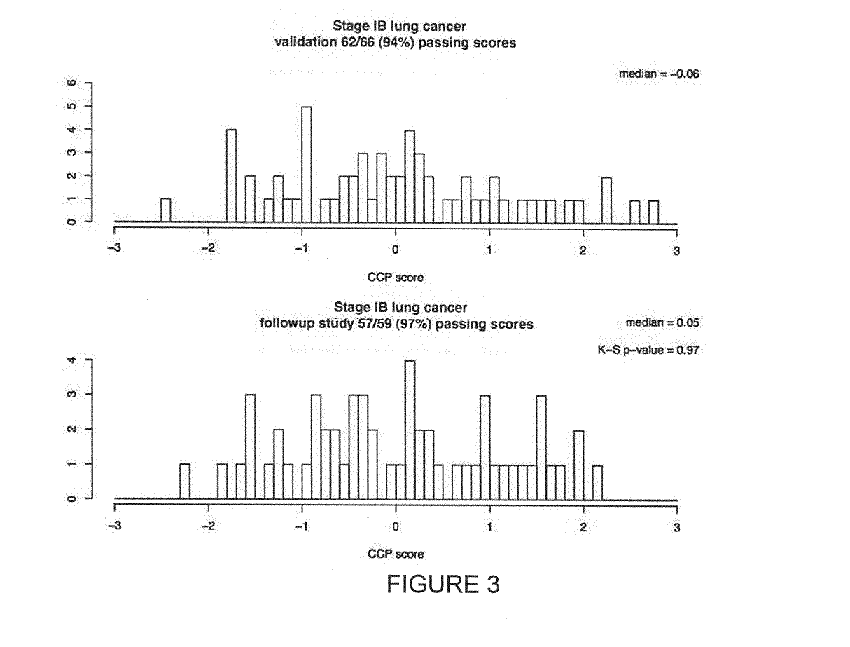

To investigate the value of the prognostic signature in stage IB, the clinically most relevant subgroup of early stage NSCLC, a survival analysis was performed in the subset of stage IB samples of set 1. A total of 66 patients were classified as stage IB of which 62 had passing CCP scores and were used for analysis. Within the stage IB subgroup the CCP score remained a significant predictor of outcome (p=0.02). Using the mean CCP score as a threshold for a high risk (above the mean) and low risk group (below the mean), two patient groups with different survival rates (95% vs 75%) could be identified (FIG. 3).

### Clinical Sample Set 2

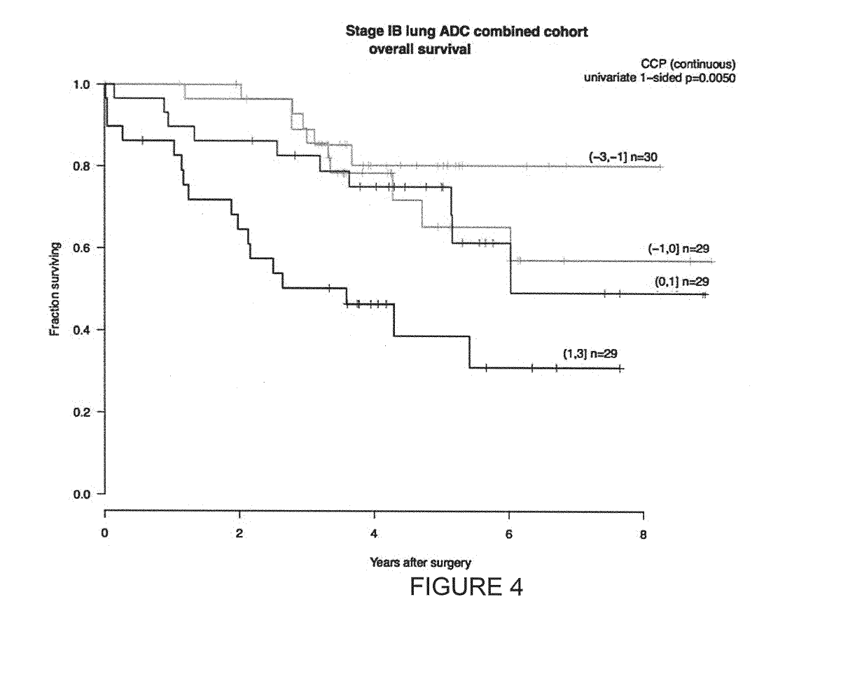

To confirm the results of the first analysis, samples were analyzed from a second, independent cohort of patients cohort ascertained between 2001 and 2005. A total of 57 samples were processed for RNA and CCP scores were determined as in the previous cohort. 55 samples received CCP scores for a passing rate of 96%. Sample quality, success rate and CCP score distribution was similar to the previous set of stage IB samples. Distribution of CCP scores in the stage IB samples from set 1 and set 2 is shown in FIG. 4. Clinical characteristics of the two IB sets was also similar except for more recent dates for surgery and follow-up dates in the second cohort. The more contemporary cohort also had a higher percentage of adjuvant treated samples (47% vs. 14%) reflecting the more aggressive use of adjuvant treatment in recent years. The percentage of smokers declined slightly compared to the older cohort (25% vs. 47%). Males were of higher risk in both cohorts, more so in the second set, but the interaction between gender and outcome was not significant after adjustment for multiple testing.

Cox proportional hazard analysis for this Set 2 stage IB cohort was performed as before. Overall survival (17 events) and disease survival (9 events) were available as outcome variables for Set 2. In univariate analysis, gender and treatment were significant predictors of overall survival and disease survival. In multivariate analysis, gender, treatment and CCP score predicted outcome. A summary of results for the two stage IB cohorts can be found in Table F (sample Set 1) and Table G (sample Set 2). In addition, tumor size (largest diameter) and pleural invasion was available for analysis. Neither parameter was significant in multivariate analysis.

TABLE F

| | |
|---|---|
| Univariate | Multivariate |

| | OS | DS | OS | DS |
|---|---|---|---|---|
| N events | 24/62 | 13/62 | 24/62 | 13/62 |
| Adjuvant | 0.18 | NA | 0.38 | NA |
| Treatment | | | | |
| Smoking Status | 0.53 | 0.64 | 0.28 | 0.7 |
| Age at Surgery | 0.19 | 0.43 | 0.1 | 0.4 |
| Gender | 0.23 | 0.35 | 0.59 | 0.94 |
| CCP (HR) | 0.02 | 0.029 | 0.029 | 0.024 |
| | (1.44) | (1.43) | (1.43) | (1.65) |

TABLE G

| | |
|---|---|
| Univariate | Multivariate |

| | OS | DS | OS | DS |
|---|---|---|---|---|
| N events | 17/55 | Sep-55 | 17/55 | Sep-55 |
| Adjuvant | 0.01 | 0.04 | 0.019 | 0.01 |
| Treatment | | | | |
| Smoking Status | 0.86 | 0.88 | 0.33 | 0.87 |
| Age at Surgery | 0.09 | 0.7 | 0.59 | 0.51 |
| Gender | 0.00009 | 0.002 | 0.002 | 0.005 |
| CCP (HR) | 0.06 | 0.19 | 0.01 | 0.09 |
| | (1.41) | (1.31) | (2.11) | (1.78) |

### Combined Stage IB Samples

To maximize statistical power both sets of stage IB samples were combined for Cox PH analysis. The results, shown in Table H, support the CCP score as a strong prognostic marker of disease outcome with a hazard ratio of 1.5 per CCP score unit.

TABLE H

| | |
|---|---|
| Univariate | Multivariate |

| | OS | DS | OS | DS |
|---|---|---|---|---|
| N events | 41/118 | 22/118 | 41/118 | 22/118 |
| Adjuvant | 0.008 | 0.027 | 0.011 | 0.0097 |
| Treatment | | | | |
| Smoking Status | 0.72 | 0.66 | 0.45 | 0.87 |
| Age at Surgery | 0.036 | 0.39 | 0.17 | 0.99 |
| Gender | 0.0006 | 0.0077 | 0.016 | 0.057 |
| Grade | 0.93 | 0.75 | NA | NA |
| CCP (HR) | 0.005 | 0.017 | 0.006 | 0.0135 |
| | (1.43) | (1.50) | (1.46) | (1.56) |

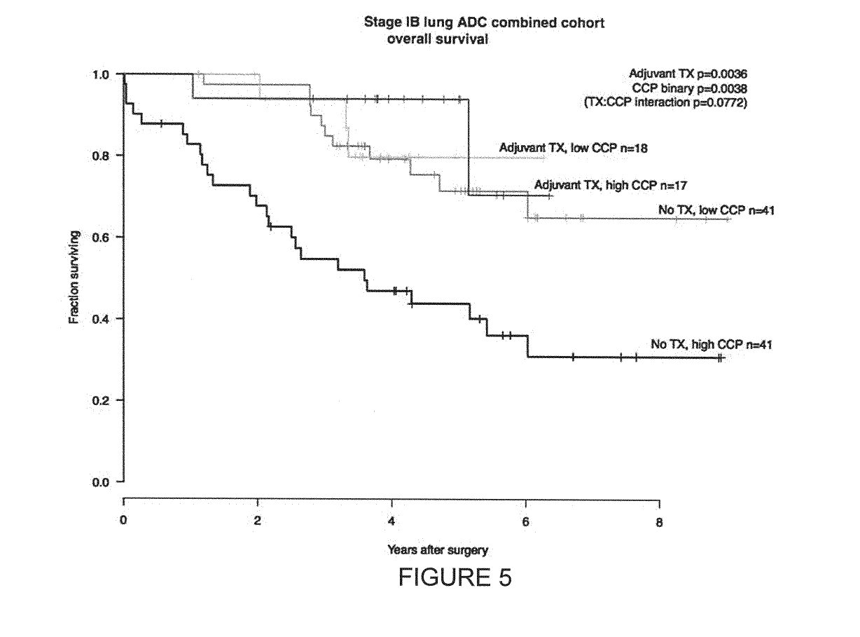

Since the distribution of CCP scores in stage IB ranges from <−2 to >2, the hazard ratio between the patient group with the lowest CCP scores and the patient set with the highest CCP levels rises to almost 7 fold. A Kaplan Meier survival analysis using CCP score quartiles (see FIG. 5) for the combined stage IB samples shows that the lowest CCP quartile has a 5-year survival rate of 80%, while the 5-year survival rate for the highest CCP score quartile drops to 30%.

### Prediction of Treatment Benefit

The RNA signature applied here as a prognostic marker in NSCLC adenocarcinoma measures the expression of proliferation genes. Chemotherapy preferentially targets rapidly proliferating cells by disrupting essential processes in the cell cycle. The inventors thus hypothesized that, in contrast to a conventional multigene panel, the CCP score not only acts as a prognostic (by identifying rapidly progressing cancers) but may also be indicative of treatment benefit (by identifying cancers that will be most susceptible to disruption of the cell cycle). The combined cohort of stage IB samples had a sufficient number of treated patients to address this question.

To test for the predictive power of the CCP score, an interaction term for CCP score and adjuvant treatment was added to the model. The test statistic is the likelihood ratio for the full model (all clinical variable, CCP score and CCP:adjuvant treatment interaction term) versus the reduced model (all clinical variables no CCP score, no interaction term). Although the interaction for CCP score and adjuvant treatment was not formally significant at the 0.05 level, it showed a strong trend (p=0.07). Most importantly, the interaction coefficient supported the assumption that high CCP scores receive more treatment benefit. A survival plot using the CCP mean as threshold within the treated and untreated sample groups in shown in FIG. 6. The Kaplan Meier plot illustrates two conclusions. First, the prognostic power of the CCP score is most pronounced in the untreated samples with a strong separation between survival rates of the high and low CCP group (high CCP 30% vs low CCP 70%). Second and possibly most unexpectedly, among the high CCP patients, patients treated adjuvantly show a much improved outcome with survival rates close to the low CCP patient group (high CCP untreated 30%, high CCP treated 70%). Thus a high CCP score correlates strongly with a higher likelihood of response to adjuvant chemotherapy (including one of the most important measures of response, i.e., survival).

### Example 2

### Introduction

This Example 2 builds on the study summarized in Example 1 above by combining the analysis in Example with analysis of additional samples. Unless indicated otherwise, all methods (e.g., sample preparation, gene expression analysis, CCP score calculation, statistical analysis, etc.) in this Example 2 were as described in Example 1. In this study, the CCP score was applied to stage I-II NSCLC ADC patients from a combined sample cohort (referred to herein as Combined Cohort) of 381 FFPE samples.

### Patient Populations

Detailed information regarding patients from the Combined Cohort is provided in Table I. The Combined Cohort was an aggregation of patient samples from two separate source cohorts, designated herein as “S1” and “S2.” S1 Cohort: 186 FFPE samples were obtained from 185 resectable stage I NSCLC ADC patients, and matching clinical data. Samples from 177 patients produced passing CCP scores. Two patients were omitted due to missing clinical data related to stage and adjuvant treatment, and one patient was omitted who died 12 days after surgery. S2 Cohort: 294 FFPE samples and 293 matching clinical records were obtained from patients with resectable non-small cell lung adenocarcinoma. 207 patients were stage I-II with passing CCP scores and complete clinical data comparable to the S1 cohort.

TABLE I

| | S1 | S2 | Total |
|---|---|---|---|
| | (N = 174) | (N = 207) | (N = 381) |
| Age mean ± SD (y) | 64 ± 8 | 66 ± 11 | 65 ± 10 |
| Sex | | | |
| Male | 122 (70%) | 94 (45%) | 216 (57%) |
| Female | 52 (30%) | 113 (55%) | 165 (43%) |
| Smoking | | | |
| Never | 26 (15%) | 34 (16%) | 60 (16%) |
| Former | 47 (27%) | 93 (45%) | 140 (37%) |
| Current | 101 (58%) | 80 (39%) | 181 (48%) |
| Stage | | | |
| IA | 120 (69%) | 64 (31%) | 184 (48%) |
| IB | 54 (31%) | 99 (48%) | 153 (40%) |
| IIA | — | 27 (13%) | 27 (7%) |
| IIB | — | 17 (8%) | 17 (4%) |
| Treatment | | | |
| Yes | 19 (11%) | 46 (22%) | 65 (17%) |
| No | 155 (89%) | 161 (78%) | 316 (83%) |
| Pleural invasion | | | |
| Yes | 24 (14%) | 80 (39%) | 104 (27%) |
| No | 150 (86%) | 127 (61%) | 277 (73%) |
| Tumor size <3 cm | | | |
| Yes | 137 (79%) | 103 (50%) | 240 (63%) |
| No | 37 (21%) | 104 (50%) | 141 (37%) |
| T stage | | | |
| T1a | 64 (37%) | 42 (20%) | 106 (28%) |
| T1b | 56 (32%) | 32 (15%) | 88 (23%) |
| T2a | 54 (31%) | 105 (51%) | 159 (42%) |
| T2b | — | 17 (8%) | 17 (4%) |
| T3 | — | 11 (5%) | 11 (3%) |
| N status | | | |
| N0 | 174 (100%) | 186 (90%) | 360 (94%) |
| N1 | — | 21 (10%) | 21 (6%) |
| Recurrence <5 y | | | |
| Yes | 36 (21%) | 55 (27%) | 91 (24%) |
| No | 138 (79%) | 152 (73%) | 290 (76%) |
| Death from disease <5 y | | | |
| Yes | 28 (16%) | 34 (16%) | 62 (16%) |
| No | 146 (84%) | 173 (84%) | 319 (84%) |

### Statistical Analysis

We evaluated the prognostic value of CCP in terms of p-values and standardized hazard ratios from univariate, and multivariate, Cox proportional hazards models. The endpoint was death from disease within five years of surgery. Death from disease was defined as death (of disease if known) following recurrence. Patients who were lost to follow-up or died of other causes were censored at the last observation.

All p-values in this report are two-sided. Univariate p-values were based on the partial likelihood ratio. Multivariate p-values were based on the partial likelihood ratio for the change in deviance from a full model (which included all relevant covariates) versus a reduced model (which included all covariates except for the covariate being evaluated, and any interaction terms involving the covariate being evaluated). In order to compare hazard ratios corresponding to different gene expression analysis platforms, hazard ratios were standardized to represent the increased risk associated with a one standard deviation increase in CCP score.

### Prognostic Information Beyond Clinical Variables

The primary goal was to further validate the results in Example 1 (i.e., CCP score adds a significant amount of prognostic information to that which is captured by conventional clinical parameters). This was accomplished by combining the CCP score with clinical variables in multivariate Cox proportional hazards models. Ideally, these models would include as many relevant clinical variables as possible. In the Combined Cohort, we were able to obtain clinical data for age, gender, smoking status, stage (7^{th} edition TNM), adjuvant treatment, pleural invasion, and tumor size. We hypothesized that the influence of adjuvant treatment might differ by stage, so we included an interaction term for stage with treatment in the cohorts where this information was available.

To measure the prognostic power of the CCP score as conservatively as possible, we coded categorical clinical variables in such a way as to explain the maximum possible variability in patient outcomes, essentially overfitting the model with clinical variables. For instance, stage was coded as a 4-level categorical variable (IA, IB, IIA, IIB) rather than a 2-level categorical variable (I,II). This resulted in less significant p-values associated with stage (due to the extra degrees of freedom, and possibly due to having fewer patients in each category), but including this extra information in a multivariate model makes it more difficult for other variables, such as CCP score, to reach significance.

### Combining FFPE Cohorts

To assess the appropriateness of combining the S1 and S2 cohorts, we tested whether clinical differences between the S1 and S2 cohorts were relevant to five year disease-related death. To this end, we constructed Cox proportional hazards models, for each of the clinical variables listed above, consisting of the clinical variable in question, a variable designating cohort, and an interaction term. After adjusting for multiple comparisons, none of the interaction terms were significant at the 5% level in two-sided likelihood ratio tests.

### Proportional Hazards and Non-Linear Effects

Plots of scaled Schoenfeld residuals versus untransformed time were used to evaluate the appropriateness of the proportional hazards assumption for these data. No evidence was found supporting time dependence for the hazard ratio of the CCP score. We also investigated the possibility that CCP score might have a non-linear effect; second- and third-order polynomials for CCP score were tested in Cox proportional hazards models but were not significant at the 5% level.

### Tests for Heterogeneity in the CCP Score Hazard Ratio

We constructed Cox proportional hazards models, for each available clinical variable, consisting of the clinical variable in question, CCP score, and an interaction term. None of these interaction terms reached significance at the 5% level.

Modeling of Variables:

Variables for each patient included age in years as a quantitative variable, gender as a binary variable (male, female), smoking status as a 3-level categorical variable (never, former, current), pathological stage (7th edition TNM classification) as a 4-level categorical variable (IA, IB, IIA, IIB), adjuvant treatment as a binary variable (no, yes), tumor size in centimeters rounded to the nearest millimeter as a quantitative variable, pleural invasion as a binary variable (no, yes), cohort as a 2-level categorical variable, and CCP score as a quantitative variable.

### Results

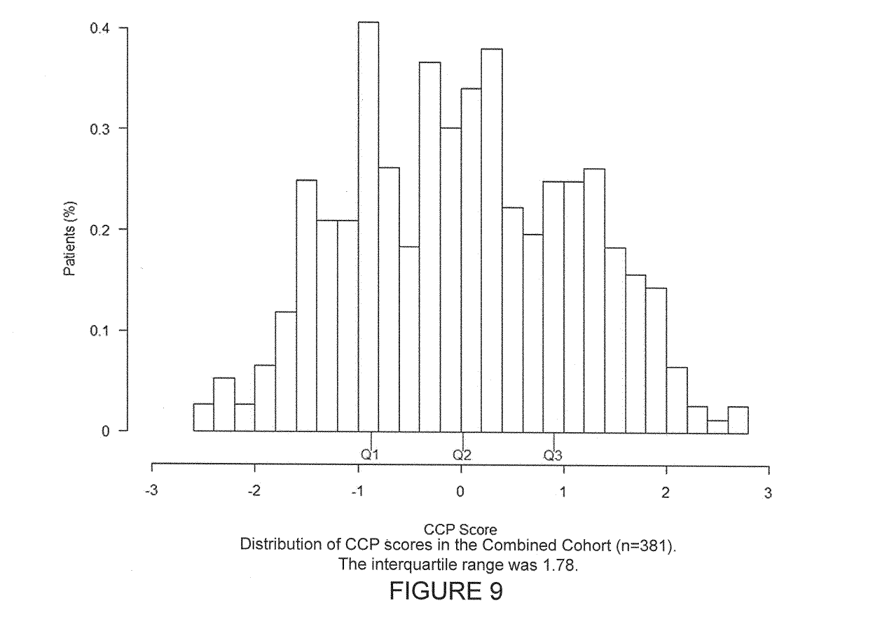

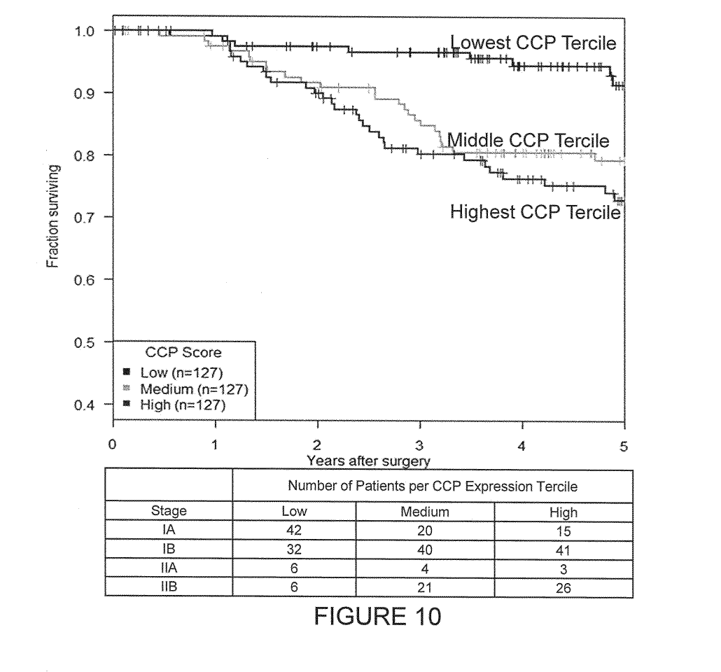

FIG. 9 shows the distribution of the CCP score among the 381 patients in the Combined Cohort. Complete results from univariate and multivariate analysis of Cox proportional hazards models are provided in Table J. In the Combined Cohort, CCP was again the most significant predictor in univariate (p-value: 0.0003) and multivariate analysis (p-value: 0.007, standardized HR: 1.50, 95% CI: 1.11-2.02). The results from multivariate analysis indicate that the CCP score was able to capture a significant amount of prognostic information independent of the many clinical variables available for the S1 and S2 cohorts. FIG. 10 shows a Kaplan-Meier plot of 5-year survival against CCP score. 5-year disease survival was 92% in patients with low CCP scores, 79% in patients with medium CCP scores, and 73% in patients with high CCP scores.

TABLE J

p-value
(unless hazard ratio indicated)

| Events/N: 62/381 | Univariate | Multivariate |
|---|---|---|
| CCP | 3.00E−04 | 7.00E−03 |
| Standardized CCP | 1.59 (1.23-2.05) | 1.5 (1.11-2.02) |
| Hazard Ratio (95% C.I.) | | |
| Age | 0.04 | 0.12 |
| Gender | 2.00E−03 | 0.01 |
| Smoking | 0.32 | 0.99 |
| Stage | 4.00E−03 | 0.15 |
| Treatment | 0.52 | 0.13 |
| Tumor Size | 7.00E−03 | 0.39 |
| Pleural Inv. | 0.01 | 9.00E−03 |
| Cohort | 0.43 | 0.61 |
| Stage:Treatment | NA | 0.09 |

### Example 3

This Example 3 builds on the study summarized in Examples 1 & 2 above by analyzing the relationship between CCP score and absolute benefit from adjuvant treatment in the S2 cohort. Unless indicated otherwise, all methods (e.g., sample preparation, gene expression analysis, CCP score calculation, statistical analysis, etc.) in this Example 3 were as described in Examples 1 & 2. Detailed information regarding patients in the S2 cohort is provided above in the description of Example 2. Of note here, the 207 addressable patients in S2 included 46 patients who had received adjuvant therapy. The treated patient set from S2 showed significant improvement (p=0.030, HR=0.32) in 5 year survival (Kaplan-Meier estimate 92.25%, 95% CI 77.70%-97.46%) compared to patients not receiving adjuvant treatment (Kaplan-Meier estimate 77.56%, 95% CI 69.46%-83.76%).

In this Example 3 it was hypothesized that the absolute benefit from adjuvant treatment (survival in treated patients minus survival in untreated patients) should be greater for patients with high CCP scores than for patients with low CCP scores. Two methods for testing this hypothesis were used. In the first method, we implemented the technique of Zhang & Klein (Confidence bands for the difference of two survival curves under the proportional hazards model, LIFETIME DATA ANALYSIS (2001)7:243-254) to evaluate the absolute difference in 5-year predicted risk of disease-related death for patients who received adjuvant treatment versus patients who did not receive adjuvant treatment over the range of observed CCP scores. In the second method, we employed complex contrast coding to test whether the absolute difference, due to treatment, in the hazard of disease related death was greater for patients with high CCP scores than for patients with low CCP scores.

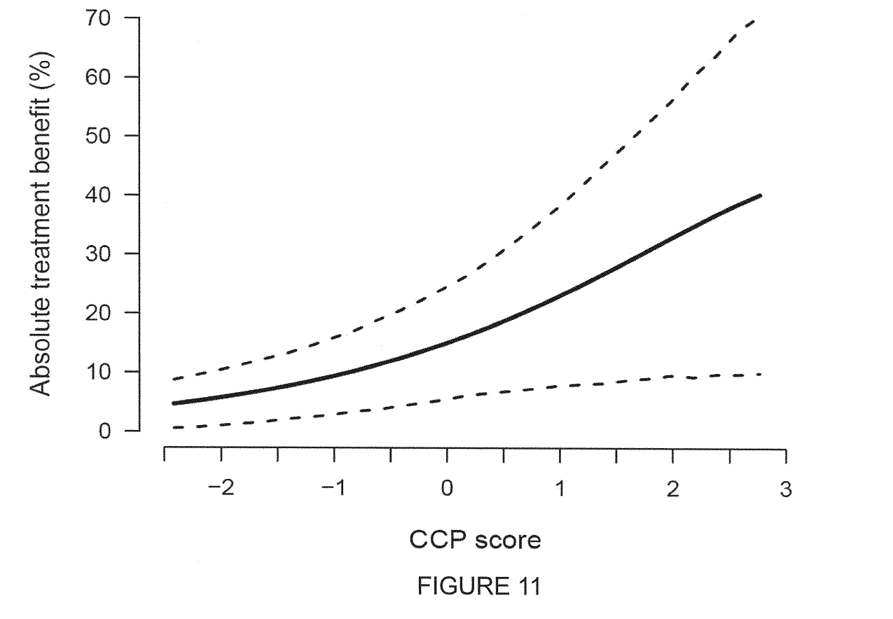

The Zhang & Klein method may be used, in particular, to test for differences in survival between two treatments (or between patients receiving treatment, and patients not receiving treatment) after adjusting for the effects of other covariates. We used this method to evaluate the difference in 5-year disease-related death between treated and untreated patients after adjusting for the effect of the CCP score. More specifically, we calculated estimates of absolute treatment benefit, together with point wise confidence bands, over the range of CCP scores observed in the S2 patient population (FIG. 11).

Contrast coding was used as follows: To test whether the absolute decrease in the hazard of disease-related death due to adjuvant treatment is significantly greater for patients with high CCP scores than for patients with low CCP scores, we categorized CCP scores as high or low using the median as the cutoff point and assigned each patient to one of four groups: high CCP with adjuvant treatment (ht), high CCP without adjuvant treatment (hu), low CCP with adjuvant treatment (lt), and low CCP without adjuvant treatment (lu). The null hypothesis

H₀:ht−hu=lt−lu,

or equivalently

H₀: ht−hu−lt+lu=0,

was tested with Cox proportional hazards regression, using 5-year disease related death as the outcome, by applying the complex contrast vector c=(1, −1, −1, 1). This analysis indicated significantly greater absolute treatment benefit for patients with high CCP scores compared to patients with low CCP scores (p=0.0060). The association between CCP score and absolute treatment benefit maintained significance after adjusting for age, gender, smoking status, stage, tumor size, and pleural invasion status in the complex contrast model (p=0.024).

### Example 4

This Example 4 builds on the study summarized in Examples 1 & 2 above by modeling and then validating a score combining CCP expression and pathological stage to assess prognosis for (predict) post-surgical risk of cancer-specific death in NSCLC patients. Unless indicated otherwise, all methods (e.g., sample preparation, gene expression analysis, CCP score calculation, statistical analysis, etc.) in this Example 4 were as described in Examples 1 & 2. Detailed information regarding patients in the S1 and S2 cohorts is provided above in the descriptions of Examples 2 & 3.

### Training

A combined prognostic score of pathological stage (pStage) and the CCP expression score was modeled in stage I and II patients without adjuvant treatment from publicly available microarray data from the Director's Consortium (DC) cohort (Shedden et al., Nat. Med. (2008) 14:822-827) and S1 and S2 of the above Examples. To adjust for platform related differences, DC values were centered by processing site and scaled by the ratio of the standard deviations of the CCP score in qPCR and microarray data. The modeling set of 495 patients included 179 patients from the DC cohort and 316 patients from the combined S1/S2 cohort. The outcome measure was five year disease-specific survival. Coefficients for the combination of CCP and pStage were derived from a bivariate Cox proportional hazards model where pathological stage was modeled as numerical variable (IA=1, IB=2, IIA=3, IIB=4). The Cox PH model was stratified by cohort. To ensure consistent contribution of each prognostic factor, all cohorts were evaluated individually. The coefficients for the final model were derived from the combination of all cohorts. The final prognostic score was scaled to represent values between 0 and 80.

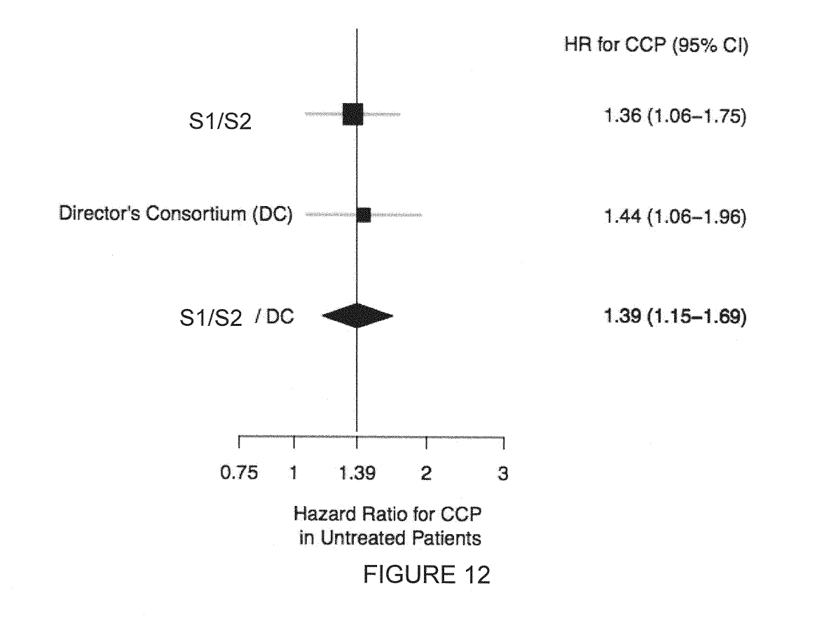

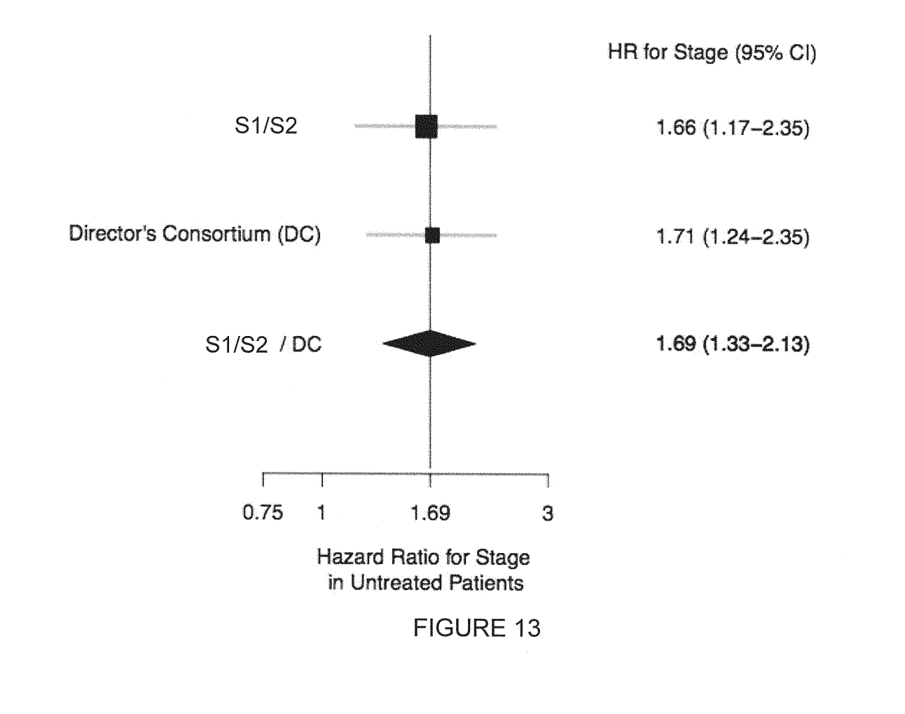

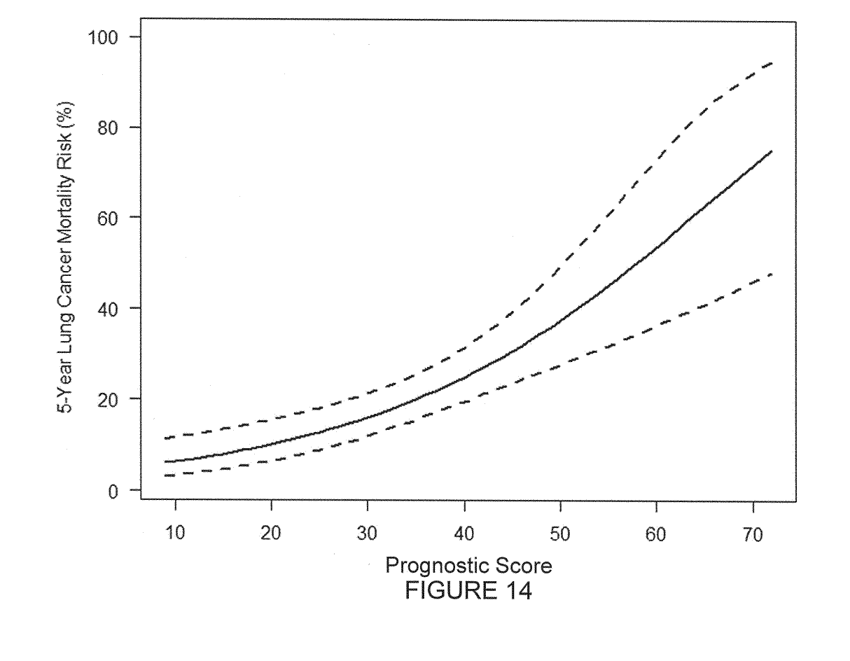

As shown in FIGS. 12 and 13, hazard ratios for CCP score and pathological stage were consistent across the various cohorts. CCP together with pathological stage provided the best prediction for lung cancer mortality, particularly according to the following formula: Prognostic score=20*(0.33*CCP score+0.52*stage)+15. FIG. 14 plots mortality risk versus combined prognostic score. Performance of CCP and pathological stage individually are shown in Table K below.

TABLE K

| Cohort | Stage HR | CCP HR | Stage | CCP |
|---|---|---|---|---|
| (Events/N) | (95% CI) | (95% CI) | p value | p value |
| S1/S2/DC | 1.69 | 1.39 | 2.7 × 10⁻⁵ | 7.8 × 10⁻⁴ |
| (90/495) | (1.33-2.13) | (1.15-1.69) | | |

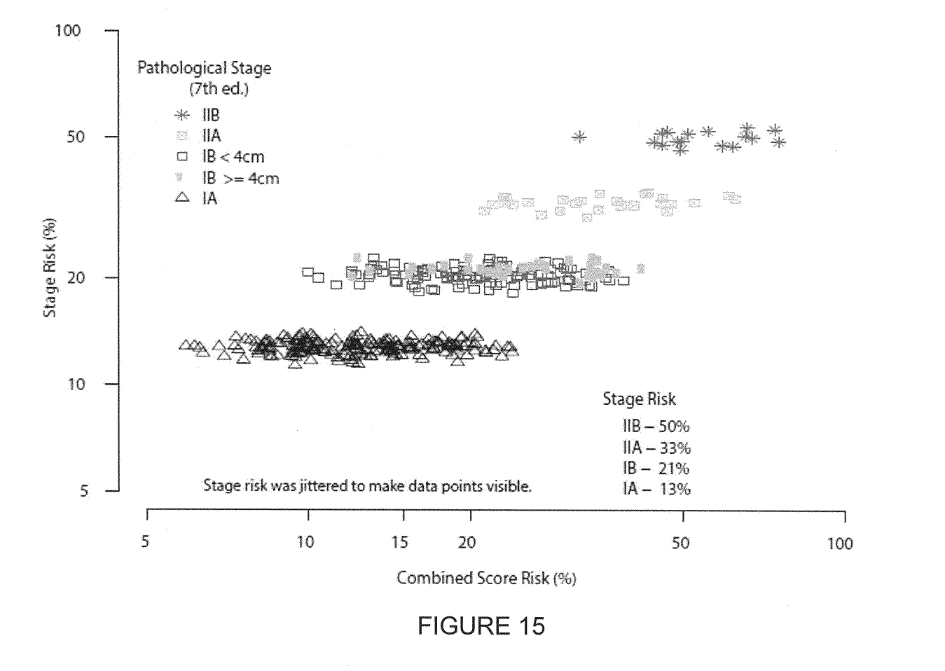

As shown in FIG. 15, the combined score differentiated 5-year lung cancer mortality risk for patients assigned the same risk based on pathological stage alone. Specifically, in the combined S1/S2 cohort, pathological stage alone provided estimates of 5-year risk of cancer-specific death of 12.6% (stage IA), 22.6% (stage IB), 38.4% (stage HA) and 60% (stage IIB). In the same cohort, the prognostic score could separate stage IA patients with 5-year risk estimates ranging from 6% to 24%. Similarly increased discrimination of risk estimates were observed for stage IB (10% to 42%), stage IIA (21% to 63%) and stage IIB patients (32% to 75%).

### Validation

Both the CCP score alone and the combined prognostic score discussed/derived above were validated in a large independent cohort. 650 patients in two cohorts (V1 and V2) aggregated for this validation met the following criteria: Stage I-II NSCLC ADC by 7th edition IASLC staging; complete surgical resection; no neo-adjuvant treatment; no adjuvant chemotherapy or radiation within 12 weeks of surgery. Characteristics of the patient cohorts are a shown in Table L below.

TABLE L

| | V1 | V2 |
|---|---|---|
| | N = 474 | N = 176 |
| | N (%) | N (%) |
| Age at Diagnosis | | |
| Median | 67 | 68 |
| SD | 11 | 10 |
| Sex | | |
| Male | 172 (36) | 69 (39) |
| Female | 302 (64) | 107 (61) |
| Tumor Size <3 cm | | |
| Yes | 394 (83) | 76 (43) |
| No | 80 (17) | 100 (57) |
| Stage | | |
| IA | 309 (65) | 36 (20) |
| IB | 142 (30) | 53 (30) |
| IIA | 15 (3) | 62 (35) |
| IIB | 8 (2) | 25 (14) |
| Pleural Invasion* | | |
| Yes | 114 (24) | 64 (36) |
| No | 343 (72) | 112 (64) |
| Disease related death at 5 y | | |
| Yes | 92 (19) | 60 (34) |
| No | 382 (81) | 116 (66) |

*Pleural invasion data were not available for 17 patients

Archived FFPE samples from surgically resected stage I-II lung adenocarcinomas were obtained and samples were processed to derive CCP scores as described in Examples 1 & 2. The pre-defined prognostic score (PS) discussed above was calculated for each patient. A PS cut-point was determined such that the percentage of stage IA patients having a PS at or below the cutpoint was close as possible to 85%, in line with published estimates of lung cancer-specific survival in stage IA patients.

Statistical analysis was performed as described above. The association of CCP, and the PS, with 5-year lung cancer mortality was evaluated using Cox proportional hazards models and likelihood ratio tests. The Mantel-Cox logrank test was used to evaluate the difference in 5-year lung cancer mortality for patients with PS scores at or below the cut-point versus patients with scores above the cut-point. All p-values are two-sided.

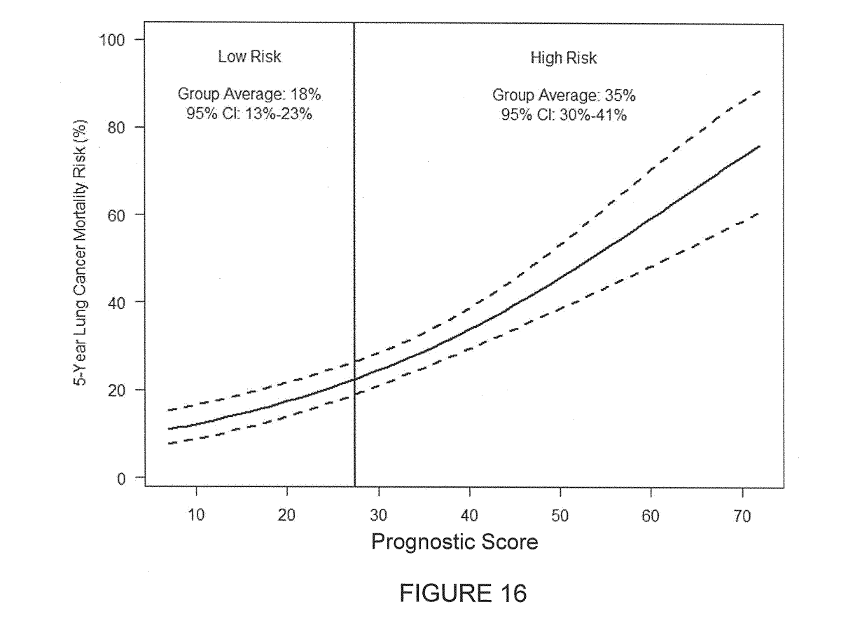

FIG. 16 shows predictions of 5 year lung cancer specific survival by PS. Low and high risk were classified by a cut-off predefined as the 85% percentile of the PS in stage IA patients. There is a significant difference between the average risk in the two patient groups.

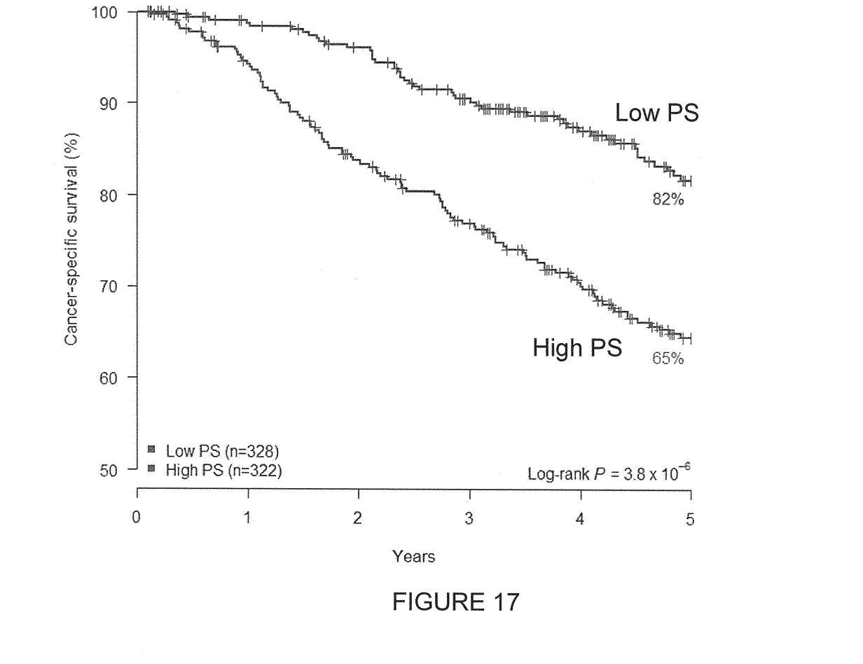

FIG. 17 shows that patients in the low PS group had significantly more favorable 5-year survival than patients in the high PS group (Log-rank P=3.8×10⁻⁷).

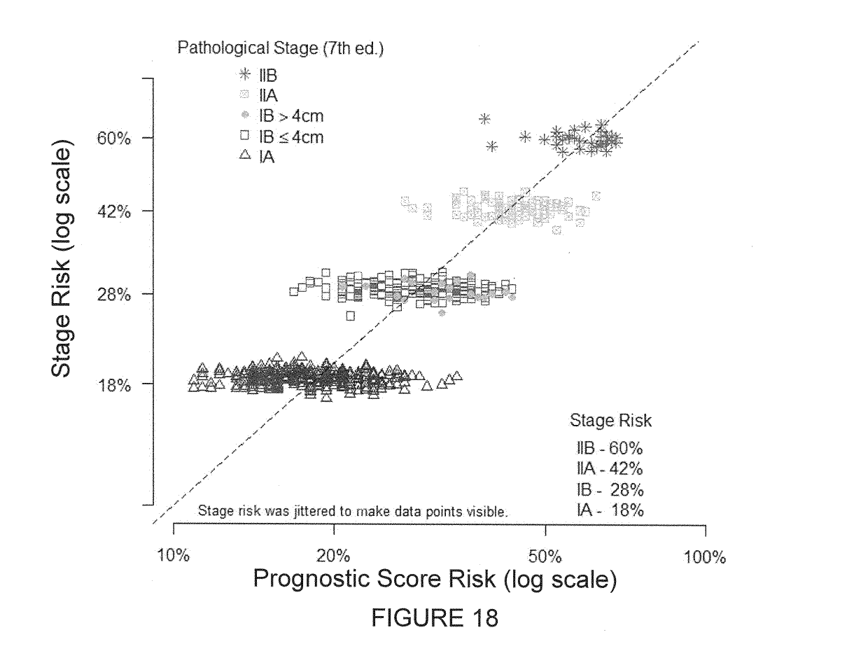

FIG. 18 shows improved risk stratification by PS compared to pathological stage alone. Specifically, the clusters of data points at 18%, 28%, 42% and 60% risk represent the percent risk of disease-specific death within 5 years for pathological stages IA, IB, HA and IIB, respectively. When pathological stage is combined with CCP score according to the model derived from the training study above, however, significantly more detailed risk can be assigned to patients who would all be assigned identical risk according to pathological stage alone. The range of risk according to PS for each pathological stage is shown by the horizontal spread of the data points in FIG. 18 and is summarized in Table M below.

TABLE M

| | Risk according to PS | | | | | |
|---|---|---|---|---|---|---|
| Pathological | | 1st | 2nd | | 3rd | |
| Stage | Minimum | quartile | quartile | Mean | quartile | Maximum |
| IA | 11% | 15% | 18% | 18% | 21% | 34% |
| IB | 17% | 25% | 29% | 29% | 33% | 43% |
| IIA | 27% | 38% | 43% | 44% | 48% | 62% |
| IIB | 38% | 54% | 61% | 59% | 64% | 68% |

Table N below provides hazard ratios and p-values showing how CCP score alone is a significant prognostic marker after adjustment for clinical variables. Results from univariate and multivariate Cox proportional hazards analysis are shown. Multivariate analysis, and univariate analysis of pleural invasion, included 633 patients with 147 events. All other univariate analyses included 650 patients with 152 events. Pleural invasion data were not available for 17 patients.

TABLE N

| | Univariate | | Multivariate | |
|---|---|---|---|---|
| | HR (95% CI) | P-Value | HR (95% CI) | P-Value |
| CCP* | 1.79 (1.42-2.27) | 1.1 × 10⁻⁶ | 1.46 (1.12-1.90) | 0.005 |
| Age | 1.02 (1.00-1.04) | 0.0097 | 1.02 (1.01-1.04) | 0.01 |
| Gender | | 0.0091 | | 0.064 |
| Male | 1 | | 1 | |
| Female | 0.65 (0.47-0.90) | | 0.73 (0.53-1.02) | |
| Stage | | 7.7 × 10⁻⁹ | | 0.0023 |
| IA | 1 | | 1 | |
| IB | 1.65 (1.11-2.44) | | 1.72 (1.00-2.96) | |
| IIA | 3.79 (2.47-5.75) | | 3.47 (1.84-6.5) | |
| IIB | 3.30 (1.76-5.77) | | 3.42 (1.28-8.62) | |
| Tumor Size# | 1.20 (1.11-1.29) | 1.1 × 10⁻⁵ | 1.01 (0.88-1.15) | 0.93 |
| Pleural | 1.30 (0.91-1.82) | 0.14 | 0.83 (0.53-1.29) | 0.41 |
| Invasion | | | | |
| Cohort | | 0.00092 | | 0.47 |
| V1 | 1 | | 1 | |
| V2 | 1.76 (1.26-2.43) | | 0.86 (0.56-1.3) | |

*Hazard ratio is reported per interquartile range of the CCP score.
#Hazard ratio is reported per cm, rounded to the nearest mm.

Table O below shows the separate prognostic value of the PS and pathological stage in univariate and bivariate models. The combination of pathological stage and CCP score into the Prognostic Score captures significant prognostic information that is not provided by pathological stage alone. Analyses included 650 patients with 152 events.

TABLE O

| | Univariate | | Bivariate | |
|---|---|---|---|---|
| | HR (95% CI) | P-Value | HR (95% CI) | P-Value |
| PS* | 2.01 (1.64-2.45) | 2.8 × 10−11 | 1.86 (1.16-2.97) | 0.0093 |
| Stage | | 7.7 × 10−9 | | 0.38 |
| IA | 1 | | 1 | |
| IB | 1.65 (1.11-2.44) | | 1.03 (0.61-1.75) | |
| IIA | 3.79 (2.47-5.75) | | 1.45 (0.62-3.35) | |
| IIB | 3.30 (1.76-5.77) | | 0.92 (0.29-2.82) | |

*Hazard ratio is reported per interquartile range of the PS score.

Table P below shows the separate prognostic value of the PS and pathological stage in univariate and bivariate models when restricted to stage IA-IB disease. The combination of pathological stage and CCP score into the Prognostic Score captures significant prognostic information that is not provided by pathological stage alone when restricted to stage IA-IB disease. Analyses included 540 patients with 101 events.

TABLE P

| | Univariate | | Bivariate | |
|---|---|---|---|---|
| | HR (95% CI) | P-Value | HR (95% CI) | P-Value |
| PS* | 1.67 (1.27-2.20) | 0.00027 | 1.74 (1.16-2.61) | 0.008 |
| Stage | | 0.012 | | 0.8 |
| IA | 1 | | 1 | |
| IB | 1.65 (1.12-2.44) | | 0.93 (0.52-1.66) | |

*Hazard ratio is reported per interquartile range of the PS score.

### Example 5

This Example 5 builds on the study summarized in Examples 1 & 2 above by combining the methods in Example 1 with analysis of additional samples, combining CCP expression and pathological stage to assess prognosis for (predict) post-surgical risk of death in patients diagnosed with lung carcinoids. Unless indicated otherwise, all methods (e.g., CCP score calculation, statistical analysis, etc.) in this Example 5 were as described in Examples 1 & 2.

In this study, CCP scores were generated as above for stage IA, IB, IIA, IIB, and IIIB lung carcinoid patients from publically available microarray data (Rousseaux et al., Ectopic Activation of Germline and Placental Genes Identifies Aggressive Metastasis-Prone Lung Cancers. Sci. Transl. Med. (2013) 186:66). Twenty-three carcinoid samples were analyzed, 11 patients with stage IA, seven patients with stage IB, 2 patients with IIA, two patients with stage IIB, and one patient with stage IIIB. The outcome measure was survival.

The association of CCP with mortality was evaluated using the Cox proportional hazards model. Results from univariate and multivariate analysis of Cox proportional hazards models are provided in Table Q. In the lung carcinoid patients, CCP was the most significant predictor in univariate and multivariate analysis.

TABLE Q

p-value

| Events/N: 5/23 | Univariate | Multivariate |
|---|---|---|
| CCP | 0.00125 | 0.0035 |
| Stage | 0.168 | 0.885 |
| Age | 0.15 | NA |

### Example 6

This Example 6 builds on the study summarized in Examples 1 & 2 above by combining the methods in Example 1 with analysis of additional samples, combining CCP expression and pathological stage to assess prognosis for (predict) post-surgical risk of death in patients diagnosed with lung carcinoids. Unless indicated otherwise, all methods (e.g., sample preparation, gene expression analysis, CCP score calculation, statistical analysis, etc.) in this Example 6 were as described in Examples 1 & 2.

In this study, CCP scores for 99 lung carcinoid samples were generated as described above. Two samples were removed because the patients died six and thirteen days after surgery, presumably from surgical complications. One sample had undefined metastasis status and was removed from the analysis. One sample was removed because it did lacked staging data, two samples were removed because they did not include clear follow-up dates, and two samples diagnosed as large-cell neruoendocrine carcinomas were removed because there were too few samples to obtain meaningful outcome analysis.

91 samples were used in the survival analysis, with 6 deaths preceded by a recurrence. Disease is spread among two histological groups: atypical (16, six recurrences, four deaths with disease), and typical (75, five recurrences, two deaths with disease). Stage was coded as a 4-level categorical variable (IA, IB, IIA/IIB, and IIIA/IIIB/IV).

The association of CCP with both death with disease, and disease free survival in lung carcinoid patients was evaluated using the Cox proportional hazards model. Results from univariate analysis of Cox proportional hazards models are provided in Table R. In the lung carcinoid patients, CCP was the most significant predictor of death with disease, and is a highly significant predictor of recurrence.

TABLE R

p-value

| | Outcome: | Outcome: |
|---|---|---|
| | death with | recurrence |
| | disease n = 91, | n = 91, |
| Variable | events = 6 | events = 11 |
| CCP | 0.0014 | 0.006 |
| Stage | 0.007 | 0.0235 |
| Histotype | 0.0018 | 0.00069 |
| Age | 0.745 | 0.286 |
| Gender | 0.093 | 0.0076 |
| Multifocal | 0.573 | 0.83 |
| Smoking | 0.318 | 0.378 |

The association of CCP and death with disease in atypical carcinoid patients alone was evaluated using the Cox proportional hazards model. CCP is a highly significant predictor of death with recurrence of disease in atypical carcinoid patients (N=14, 4 events, p-value 0.0102).

All publications and patent applications mentioned in the specification are indicative of the level of those skilled in the art to which this invention pertains. All publications and patent applications are herein incorporated by reference to the same extent as if each individual publication or patent application was specifically and individually indicated to be incorporated by reference. The mere mentioning of the publications and patent applications does not necessarily constitute an admission that they are prior art to the instant application.

Although the foregoing invention has been described in some detail by way of illustration and example for purposes of clarity of understanding, it will be obvious that certain changes and modifications may be practiced within the scope of the appended claims.

1. An in vitro method of classifying lung cancer comprising:
(1) measuring in a sample the expression of a panel of biomarkers comprising at least four CCP biomarkers chosen from the group consisting of DLGAP5, ASPM, KIF11, BIRC5, CDCA8, CDC20, MCM10, PRC1, BUB1B, FOXM1, NUSAP1, C18orf24, PLK1, CDKN3, RRM2, RAD51, CEP55, ORC6L, RAD54L, CDC2, CENPF, TOP2A, KIF20A, KIAA0101, CDCA3, ASF1B, CENPM, TK1, PIM, PTTG1 and DTL;
(2) providing a test value by
(a) weighting the determined expression of each of a plurality of test biomarkers selected from the panel of biomarkers with a predefined coefficient, wherein said plurality of test biomarkers comprises said at least four CCP biomarkers; and
(b) combining the weighted expression to provide the test value, wherein the combined weight given to said at least four CCP biomarkers is at least 40% of the total weight given to the expression of said plurality of test biomarkers; and
(3) correlating said test value to
(a) an unfavorable classification if said test value reflects high expression of the plurality of test biomarkers; or
(b) a favorable classification if said test value reflects low or normal expression of the plurality of test biomarkers.

2. The method of claim 1, wherein at least 75% of said plurality of test biomarkers are chosen from the group consisting of DLGAP5, ASPM, KIF11, BIRC5, CDCA8, CDC20, MCM10, PRC1, BUB1B, FOXM1, NUSAP1, C18orf24, PLK1, CDKN3, RRM2, RAD51, CEP55, ORC6L, RAD54L, CDC2, CENPF, TOP2A, KIF20A, KIAA0101, CDCA3, ASF1B, CENPM, TK1, PBK, PTTG1 and DTL.

3. The method of claim 1, wherein said panel of biomarkers and said plurality of test biomarkers each comprise the top 3 genes in Table 5.

4. The method of claim 1, wherein said panel of biomarkers and said plurality of test biomarkers each comprise the biomarkers in Panel F.

5. The method of claim 1, wherein said unfavorable classification is chosen from the group consisting of (a) a poor prognosis, (b) an increased likelihood of cancer progression, (c) an increased likelihood of cancer recurrence, (d) an increased likelihood of cancer-specific death, or (e) a decreased likelihood of response to treatment with a particular regimen.

6. The method of claim 5, wherein said unfavorable classification is an increased likelihood of cancer-specific death.

7. The method of claim 5, wherein said unfavorable classification is a decreased likelihood of response to treatment comprising chemotherapy.

8. The method of claim 1, wherein said favorable classification is chosen from the group consisting of (a) a good prognosis, (b) no increased likelihood of cancer progression, (c) no increased likelihood of cancer recurrence, (d) no increased likelihood of cancer-specific death, or (e) an increased likelihood of response to treatment with a particular regimen.

9. The method of claim 8, wherein said favorable classification is no increased likelihood of cancer-specific death.

10. The method of claim 8, wherein said favorable classification is an increased likelihood of response to treatment comprising chemotherapy.

11-18. (canceled)

19. A method of treating cancer in a patient having lung cancer, comprising:
determining in a sample from said patient the expression of a panel of genes in said sample including at least 4 CCGs;
providing a test value by (1) weighting the determined expression of each of a plurality of test genes selected from said panel of genes with a predefined coefficient, and (2) combining the weighted expression to provide said test value, wherein at least 60% or 75% of said plurality of test genes are CCGs, wherein an increased level of expression of said plurality of test genes indicates a poor prognosis and/or an increased likelihood of response to a treatment regimen comprising chemotherapy; and
administering to said patient an anti-cancer drug, or recommending or prescribing or initiating a treatment regimen comprising chemotherapy based at least in part on whether a poor prognosis and/or an increased likelihood of response to a treatment regimen comprising chemotherapy is indicated.

20. A kit for prognosing cancer in a patient having lung cancer and/or for determining the likelihood of response to a treatment regimen comprising chemotherapy, comprising, in a compartmentalized container:
a plurality of PCR primer pairs for PCR amplification of at least 5 test genes, wherein less than 10%, 30% or less than 40% of all of said at least 8 test genes are non-CCGs; and
one or more PCR primer pairs for PCR amplification of at least one housekeeping gene.

21-33. (canceled)

34. A system for prognosing cancer in a patient having lung cancer and/or for determining the likelihood of response to a treatment regimen comprising chemotherapy, comprising:
(1) a sample analyzer for determining the expression levels of a panel of genes including at least 4 CCGs in a sample from said patient, wherein the sample analyzer contains the tumor sample, RNA expressed from the panel of genes, or DNA synthesized from such RNA; and
(2) a first computer subsystem programmed for (a) receiving gene expression data on at least 4 test genes selected from the panel of genes, (b) weighting the determined expression of each of the test genes, and (c) combining the weighted expression to provide a test value, wherein the combined weight given to said at least 4 CCGs is at least 40% of the total weight given to the expression of all of said plurality of test genes; and
(3) a second computer subsystem programmed for comparing the test value to one or more reference values each associated with a predetermined prognosis and/or a predetermined likelihood of response to the particular treatment regimen.

35. The system of claim 34, further comprising a display module displaying the comparison between the test value to the one or more reference values, or displaying a result of the comparing step.

36. The method of claim 1, wherein said CCGs are the top 2, 3, 4, 5, 6, 7, 8, 9, 10, 11, 12, 13, 14, 15, 20, 25, 30, 35, 40 genes listed in any of Tables 5, 6, 7, 10, 11, 12, 13, 14, 15, 16, 17, 18, 19, 20, 21, 22, or 23.

37. The kit of claim 20, wherein said CCGs are the top 2, 3, 4, 5, 6, 7, 8, 9, 10, 11, 12, 13, 14, 15, 20, 25, 30, 35, 40 genes listed in any of Tables 5, 6, 7, 10, 11, 12, 13, 14, 15, 16, 17, 18, 19, 20, 21, 22, or 23.

38. (canceled)

39. The system of claim 34, wherein said CCGs are the top 2, 3, 4, 5, 6, 7, 8, 9, 10, 11, 12, 13, 14, 15, 20, 25, 30, 35, 40 genes listed in any of Tables 5, 6, 7, 10, 11, 12, 13, 14, 15, 16, 17, 18, 19, 20, 21, 22, or 23.

40-47. (canceled)
